(19) United States
(12) Patent Application Publication
(10) Pub. No.: US 2010/0305103 A1
(43) Pub. Date: Dec. 2, 2010

(54) GENES DIFFERENTIALLY EXPRESSED IN BIPOLAR DISORDER AND/OR SCHIZOPHRENIA

(75) Inventors: Marquis P. Vawter, Laguna Niguel, CA (US); Ling Shao, Irvine, CA (US)

Correspondence Address: Weaver Austin Villeneuve & Sampson LLP, P.O. BOX 70250, OAKLAND, CA, 94612-0250, US

(73) Assignee: THE REGENTS OF THE UNIVERSITY OF CALIFORNIA, Oakland, CA (US)

(21) Appl. No.: 12/779,854

(22) Filed: May 13, 2010

Related U.S. Application Data
(63) Continuation of application No. 11/712,827, filed on Feb. 28, 2007, now abandoned.
(60) Provisional application No. 60/840,248, filed on Aug. 25, 2006.
(60) Provisional application No. 60/777,945, filed on Feb. 28, 2006.

(51) Int. Cl. C12Q 1/68; A61K 31/5415; A61K 31/454; A61K 31/496; A61K 31/451; A61K 31/46; A61K 31/13; A61K 31/335; A61K 31/138; A61P 25/18; A61P 25/24

(52) U.S. Cl. 514/217; 435/6; 514/225.8; 514/323; 514/252.13; 514/226.2; 514/225.5; 514/327; 514/304; 514/317; 514/662; 514/450; 514/253.04; 514/652

(57) ABSTRACT

This invention provides molecular markers that are prognostic and/or diagnostic for a psychiatric disorder. In particular, genes are identified whose expression is altered in schizophrenia and/or bipolar disorder thereby providing prognostic and diagnostic markers for the disorder. In addition genes are identified whose dysregulation provides markers that allow diagnostic distinction between schizophrenia and bipolar disorder.

### CROSS-REFERENCE TO RELATED APPLICATIONS

This application is a Continuation of U.S. Ser. No. 11/712,827, filed on Feb. 28, 2007 which claims benefit of and priority to U.S. Ser. No. 60/840,248, filed on Aug. 25, 2006, and U.S. Ser. No. 60/777,945, filed on Feb. 28, 2006, all of which are incorporated herein by reference in their entirety for all purposes.

### STATEMENT AS TO RIGHTS TO INVENTIONS MADE UNDER FEDERALLY SPONSORED RESEARCH AND DEVELOPMENT

This work was supported by Federal Research Grant No: MH74307A. The Government of the United States of America has certain rights in this invention.

### FIELD OF THE INVENTION

This invention pertains to the field of psychiatric diagnostics. In particular, molecular markers are provided that are good markers for bipolar disorder and/or schizophrenia.

### BACKGROUND OF THE INVENTION

Schizophrenia and bipolar disorder have been traditionally diagnosed by clinical examination of psychotic symptoms and affective dysregulation. The clinical impressions along these two dimensions coupled with historical separation into current diagnostic classifications have led to these illnesses being viewed and treated in research as independent classes (Craddock et al. (2005) J Med Genet 42(3): 193-204; Craddock and Owen (2005) Br. J. Psychiatry 186: 364-366). However, it has not escaped attention that these nomothetic classifications share some pathophysiology, vulnerability and risk factors, genetic loci, clinical manifestations, and approximate ages of onset. Medication response can be effective in one or both disorders or equally ineffective in both disorders. Categorization into separate classes has led to efforts for identification of separate pathophysiology for each disorder (Craddock et al. (2006) Schizophr Bull., 32(1): 9-16).

### SUMMARY OF THE INVENTION

This invention pertains to the discovery/identification of common molecular profiles for both schizophrenia and/or bipolar disorder, as well as molecular profiles that can be used to distinguish these conditions (e.g., as indicators in a differential diagnosis).

In certain embodiments this invention provides methods of detecting the presence of, or a predisposition to, a psychiatric illness in a human. The methods typically involve providing a biological sample from the human (e.g., psychiatric patient); and screening the biological sample for increased or decreased expression of two or more genes listed in Table 6, where upregulation or downregulation (as indicated in Table 6) of expression of the two or more genes, as compared to a control, is an indicator for the presence of, or predisposition to, a psychiatric illness.

Thus, in certain embodiments this invention provides methods of detecting the presence of, or a predisposition to, a psychiatric illness in a human. The methods typically involve screening a biological sample from the human for increased or decreased expression of at least one, and in certain embodiments, two or more genes listed in Table 6 (and/or one or more of Tables 1, 2, 9, and/or 10) where upregulation or downregulation (e.g., as indicated in the respective table, e.g., Table 6) of expression of the two or more genes, is an indicator for the presence of, or predisposition to, a psychiatric illness. In certain embodiments the psychiatric illness is schizophrenia and/or bipolar disorder. In certain embodiments the two or more genes comprises two or more, or three or more, or four or more, or five or more, or six or more, or seven or more, or eight or more, or nine or more, or 10 or more, or 11 or more, or 12 or more, or 13 or more, or 14 or more, or 15 genes selected from the group consisting of BUB1B, ERBB2, FGF2, FTH1, IL2RA, LGALS3, MT1X, NFATC1, OGDH, PPARA, PVR, SOX9, SSPN, TXNIP, and UNG, and/or one or more or two or more or three or more, or four or more, or five or more, or six or more, or seven or more or eight genes selected from the group consisting of EMX2, ERBB2, FGF2, JARID2, RAB23, SMO, SOX9, and THBS4. In certain embodiments the two or more genes comprises EMX2, ERBB2, FGF2, JARID2, RAB23, SMO, SOX9, and THBS4. In certain embodiments the two or more genes comprises two or more genes, or three or more, or four or more, or five or more, or six or more, or seven or more, or eight or more, or nine or more, or 10 genes selected from the group consisting of AGXT2L1, EMX2, SOX9, TU3A, TUBB2B, IMPA2, SLC1A2, GMPR, AHNAK, and ATP6V1H. In certain embodiments the two or more genes comprises two or more genes, or three or more, or four or more, or five or more, or six or more, or seven or more, or eight or more, or nine or more, or 10 or more, or 11 or more, or 12 or more, or 13 or more, or 14 or more, or 15 genes selected from the group consisting of BUB1 B, EMX2, ERBB2, FGF2, FTH1, IL2RA, LGALS3, MAFG, NFATC1, PVR, RERG, SMCY, SMO, SOX9, TXNIP. In various embodiments the two or more genes comprises two or more genes comprises two or more genes, or three or more, or four or more, or five or more, or six or more, or seven or more, or eight or more, or nine or more, or 10 genes selected from the group consisting of CASP6, EPHB4, GLUL, HMGB2, MAOA, NOTCH2, SLC1A3, SLC6A8, TNFSF10, TNFSF8. In various embodiments the two or more genes comprises two or more, or three or more, or four genes selected from the group consisting of HOMER1, MCCC2, CORT, and RGS4. In certain embodiments the two or more genes comprises two or more, or three genes selected from the group consisting of ATP6V1D, GSR, and SH3GLB1, and/or two or more, or three genes selected from the group consisting of PPP1R3C, CYP4F11, and SCEL. In certain embodiments the screening comprises screening the biological sample for increased or decreased expression of three or more genes, five or more, 10 or more, 15 or more, 20 or more, 25 or more, 30 or more, 40 or more, 50 or more, 60, or more, 70 or more, or all of the genes listed in Table 6.

In certain embodiments, this invention provides methods of distinguishing between schizophrenia and bipolar disorder or between a predisposition to schizophrenia and a predisposition to bipolar disorder in a human. The methods typically involve screening a biological sample from the human for increased or decreased expression of one, preferably two or more, three or more, four or more, five or more, 10 or more, (and so forth) genes listed in Table 1, and/or Table 10, and/or Table 2, and/or Table 9, where dysregulation of the expression of the gene(s) as indicated in Table 1 or Table 10, as compared to a control, indicates the presence of, or a predisposition to schizophrenia, and dysregulation of the expression of the gene(s) as indicated in Table 2 or Table 9, as compared to a control, indicates the presence of or a predisposition to bipolar disorder. In certain embodiments the screening comprises screening the biological sample for increased or decreased expression of two or more genes, or three genes selected from the group consisting of ATP6V1D, GSR, and SH3GLB1, and/or two or more genes, or three genes selected from the group consisting of PPP1R3C, CYP4F11, and SCEL.

In various embodiments, of the assays described above, the screening comprises screening genes whose expression is concordant in DLPFC and lymphocytes. In various embodiments of these assays, the biological sample comprises a lymphocyte and/or a neurological tissue. In various embodiments of these assays, the human is a human undergoing psychiatric evaluation. In various embodiments of these assays, the human is a human receiving psychoactive medication. In various embodiments of these assays, the human is a child or an adolescent. In various embodiments of these assays, the human is an adult. In various embodiments of these assays, the screening comprises a nucleic acid hybridization to determine an mRNA level of the gene(s). Thus, for example, the determining can comprise a method selected from the group consisting of a Northern blot, a Southern blot using DNA derived from an RNA expressed by the two or more genes, an array hybridization, an affinity chromatography, an RT-PCR using an RNA expressed by the two or more genes, and an in situ hybridization. In various embodiments of these assays, the determining method involves an array hybridization using a high density nucleic acid array (e.g., an Affymetrix array). In various embodiments of these assays, the determining involves an array hybridization using a spotted array. In various embodiments of these assays, the determining involves a real time quantitative PCR (RT-QPCR) using a DNA reverse transcribed from mRNA expressed by the genes as a template. In various embodiments the screening comprises detecting a protein(s) expressed by the two or more genes. For example, the protein can be detected via a method selected from the group consisting of capillary electrophoresis, a Western blot, mass spectroscopy, ELISA, immunochromatography, and immunohistochemistry. In various embodiments of these assays, the upregulation or downregulation is with respect to a control comprising baseline levels of expression determined for a members of a normal healthy population. In various embodiments of these assays, the upregulation or downregulation is with respect to a control comprising levels of expression determined for the human at an earlier time.

Also provided are methods of treating a human subject for a psychiatric disorder. The methods typically involve utilizing a biological sample from the human subject to detect the presence of or predisposition to a psychiatric illness in a the human according to the methods described herein; and prescribing or providing more aggressive therapy for the human subject if the human subject tests positive for the presence or predisposition to a psychiatric illness; and/or prescribing treatment for schizophrenia for if the human subject tests positive for the presence or predisposition to schizophrenia, and/or or prescribing treatment for bipolar disorder for if the human subject tests positive for the presence or predisposition to bipolar disorder. In certain embodiments the prescribing or providing comprises providing cognitive therapy to the subject. In certain embodiments the prescribing or providing comprises prescribing psychoactive medication for the subject. In certain embodiments the prescribing or providing comprises prescribing psychoactive medication for the subject where the psychoactive medication is selected from the group consisting of Neuroleptics (antipsychotics), antiparkinsonian agents, sedatives and anxiolytics, antidepressants, a mood stabilizer, and an anticonvulsant drug. In certain embodiments the medication comprises a neuroleptic selected from the group consisting of trifluoperazine (Stelazine), pimozide (Orap), flupenthixol (Fluanxol), and chlorpromazine (Largactil), flupenthixol (Fluanxol), fluphenazine decanoate (Modecate), pipotiazine (Piportil L4), and haloperidol decanoate (Haldol LA). In certain embodiments the medication comprises an antiparkinsonian agent selected from the group consisting of benztropine mesylate (Cogentin), trihexyphenidyl (Artane), procyclidine (Kemadrin), and amantadine (Symmetrel). In certain embodiments the medication comprises a sedative and/or anxiolytic selected from the group consisting of a barbiturate, a benzodiazepine, and a non-barbiturate sedative. In certain embodiments the medication comprises an antidepressant selected from the group consisting of a tricyclic (e.g., amitriptyline (Elavil), imipramine (Tofranil), doxepin (Sinequan), clomipramine (Anafranil)), a monoamine oxidase inhibitors (e.g., phenelzine (Nardil) and tranylcypromine (Parnate)), a tetracyclic (e.g. maprotiline (Ludiomil)), trazodone (Desyrel) and fluoxetine (Prozac). In certain embodiments the medication comprises a mood stabilizer selected from the group consisting of lithium and carbamazepine.

In various embodiments this invention provides methods of screening for an agent that mitigates one or more symptoms of a psychiatric disorder. The methods typically involve administering a test agent to a cell and/or a mammal; and detecting altered expression in the cell and/or mammal of one, or two or more, or three or more, or five or more, or 10 or more (and so forth) genes listed in Tables 1, 2, 6, 9, or 10, where upregulation or downregulation (as indicated in Tables 1, 2, 6, 9, or 10) of expression of the two or more genes, e.g., as compared to a control, is an indicator that the test agent has activity that mediates one or more symptoms of a psychiatric disorder. In certain embodiments the psychiatric illness is schizophrenia and/or bipolar disorder.

In certain embodiments the two or more genes comprises two or more, or three or more, or four or more, or five or more, or six or more, or seven or more, or eight or more, or nine or more, or 10 or more, or 11 or more, or 12 or more, or 13 or more, or 14 or more, or 15 genes selected from the group consisting of BUB1B, ERBB2, FGF2, FTH1, IL2RA, LGALS3, MT1X, NFATC1, OGDH, PPARA, PVR, SOX9, SSPN, TXNIP, and UNG, and/or one or more or two or more or three or more, or four or more, or five or more, or six or more, or seven or more or eight genes selected from the group consisting of EMX2, ERBB2, FGF2, JARID2, RAB23, SMO, SOX9, and THBS4. In certain embodiments the two or more genes comprises EMX2, ERBB2, FGF2, JARID2, RAB23, SMO, SOX9, and THBS4. In certain embodiments the two or more genes comprises two or more genes, or three or more, or four or more, or five or more, or six or more, or seven or more, or eight or more, or nine or more, or 10 genes selected from the group consisting of AGXT2L1, EMX2, SOX9, TU3A, TUBB2B, IMPA2, SLC1A2, GMPR, AHNAK, and ATP6V1H. In certain embodiments the two or more genes comprises two or more genes, or three or more, or four or more, or five or more, or six or more, or seven or more, or eight or more, or nine or more, or 10 or more, or 11 or more, or 12 or more, or 13 or more, or 14 or more, or 15 genes selected from the group consisting of BUB1B, EMX2, ERBB2, FGF2, FTH1, IL2RA, LGALS3, MAFG, NFATC1, PVR, RERG, SMCY, SMO, SOX9, TXNIP. In various embodiments the two or more genes comprises two or more genes comprises two or more genes, or three or more, or four or more, or five or more, or six or more, or seven or more, or eight or more, or nine or more, or 10 genes selected from the group consisting of CASP6, EPHB4, GLUL, HMGB2, MAOA, NOTCH2, SLC1A3, SLC6A8, TNFSF10, TNFSF8. In various embodiments the two or more genes comprises two or more, or three or more, or four genes selected from the group consisting of HOMER1, MCCC2, CORT, and RGS4. In certain embodiments the two or more genes comprises two or more, or three genes selected from the group consisting of ATP6V1D, GSR, and SH3GLB1, and/or two or more, or three genes selected from the group consisting of PPP1R3C, CYP4F11, and SCEL. In certain embodiments the screening comprises screening the biological sample for increased or decreased expression of three or more genes, five or more, 10 or more, 15 or more, 20 or more, 25 or more, 30 or more, 40 or more, 50 or more, 60, or more, 70 or more, or all of the genes listed in Table 6. In various embodiments, the screening comprises screening genes whose expression is concordant in DLPFC and lymphocytes. In various embodiments, the screening comprises screening genes whose altered expression is predominant in neurological tissue. In various embodiments the expression pattern is detected by measuring RNA expression levels or detecting/quantifying translated protein, e.g., as described herein. In certain embodiments the control comprises a cell contacted or mammal not treated with the test agent or treated with the test agent at a lower concentration. In certain embodiments the test agent is not an antibody and/or not a protein. In certain embodiments the test agent is a small organic molecule. In certain embodiments the cell is cultured ex vivo.

Where reference is made to two or more genes in a Table, in various embodiments, this invention contemplates any combination of two or more, three or more, four or more and so forth up to the entire list of genes in that Table.

In various embodiments specific genes that are particularly useful for diagnostic/prognostic markers include, but are not limited to claims, would be bipolar disorder specific genes that are concordant in brain and lymphocytes (see, e.g., ATP6V1D, GSR, SH3GLB1, and the like), and/or schizophrenia specific genes that are concordant in brain and lymphocytes (see, e.g., PPP1R3C, CYP4F11, SCEL, and the like.).

In certain embodiments, genes whose expression pattern is discordant in DLPFC and lymphocytes are excluded as prognostic/diagnostic markers (see, e.g., genes labeled opposite in Tables 9 and 10.

In certain embodiments specific genes that are brain relevant include, but are not limited to brain-specific, (e.g., or selectively enriched in brain tissues), highly correlated in expression, and are differentially expressed in both schizophrenia and bipolar disorders (see, e.g., AGXT2L1, TU3A, TUBB2B, SOX9, ATP6V1H, GMPR, EMX2, AHNAK, IMPA2, SLC1A2, and the like). These genes form one illustrative set of screening candidates for use, for example, in human cell lines and animal models derived from central nervous system tissues. Dysregulation of these markers in peripheral biomarker screening assays may be low due to low expression in peripheral tissues, but can be more accurately analyzed with more sensitive techniques. Marker genes such as these provide relevant targets for compound screening for therapeutics.

In certain embodiments the methods of this invention expressly exclude monitoring expression of one or more of the following genes: neuregulin 1 (NRG1), FTH1, KIAA0515, KIAA0020, CFC1, SMCY, RAB23, BUB1B, IL2RA, and/or one or more of the following genes: IMPA2, SLC1A2, FGF2, ERBB2, MDH1, GMPR, PPARA. In certain embodiments methods of this invention expressly exclude monitoring expression of all of the following genes: FTH1, KIAA0515, KIAA0020, CFC1, SMCY, RAB23, BUB1B, IL2RA, and/or all of the following genes: IMPA2, SLC1A2, FGF2, ERBB2, MDH1, GMPR, PPARA.

### DEFINITIONS

The phrase “dysregulation of the expression of the gene(s) as indicated in Table XX” or “altered expression of the gene(s) as indicated in Table XX”, where XX is the Table number indicates that the expression of the gene(s) is upregulated or downregulated as shown in the table or expression level is not significantly altered as shown in the table. It is not required that the expression levels match those shown in the table, simply when the table shows upregulation of expression of the gene(s) is associated with a particular condition, then measured upregulation of expression of those gene(s) in a subject it taken as an indicator of that condition, and when the table shows that downregulation of expression of the gene(s) is associated with a particular condition, then measured downregulation of expression of those gene(s) in a subject it taken as an indicator of that condition. In various embodiments, the measured upregulation of expression or downregulation of expression is a significant upregulation or downregulation, preferably a statistically significant upregulation or down regulation (e.g., at the 90% or greater, preferably 95% or greater, more preferably 98% or greater or 99% or greater confidence level). In certain embodiments, the upregulation or downregulation is at least 10%, 20%, 25%, or 30%, more preferably at least 50%, 75% or 90%. In certain embodiments, the upregulation is at least 100%, 125% 150%, 200%, 300%, 400%, or 500%. In various embodiments, the change in expression level is at least 1.25 fold, preferably at least 1.5 fold, more preferably at last 2 fold, at least 4, fold, or at least 10 fold.

The phrase “increased or decreased expression” when used with respect to one or more genes indicates increased or decreased levels of mRNA transcript of said genes. This can be produced by increased or decreased regulation of transcription and/or alterations of copy number of the gene(s). Increased or decreased expression is typically with respect to a reference transcription level (e.g., a control). Illustrative controls include, but are not limited to the transcription levels found in a “normal healthy” population (e.g., a healthy population having the same age and/or gender) and/or the same transcription level found in the same subject at a different time (e.g., at a earlier time of life) and/or the transcription level found in one or more “reference” genes.

The term “indicator” when used, e.g. in a diagnostic assay (i.e., when a factor is said to be an indicator of a psychiatric disorder) need not require that the measured factor be dispositive of the presence or absence of the disorder or dispositive of the future occurrence of the disorder. The factor can simply indicate a predisposition to the disorder (e.g., a greater likelihood of presence or future occurrence of the disorder than is found in the absence of the indicator). It will be appreciated that such an indicator can be one of a number of indicators used, typically in a differential diagnosis for the disease/disorder.

The phrase “significant”, when used with respect to upregulation or downregulation of gene expression preferably refers to statistically significant (e.g. at the 90%, preferably 95%, more preferably at least at the 98% or 99% confidence level).

The term “gene product” refers to a molecule that is ultimately derived from a gene. The molecule can be a polypeptide encoded by the gene, an mRNA encoded by a gene, a cDNA reverse transcribed from the mRNA, and so forth.

The phrase “expression or activity of a gene” refers to the production of a gene product (e.g. the production of an mRNA and/or a protein) or to the activity of a gene product (i.e., the activity of a protein encoded by the gene).

The term “expression” refers to protein expression, e.g., mRNA and/or translation into protein. The term “activity” refers to the activity of a protein. Activities include but are not limited to phosphorylation, signaling activity, activation, catalytic activity, protein-protein interaction, transportation, etc. The expression and/or activity can increase, or decrease. Expression and/or activity can be activated directly or indirectly.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The term “antibody”, as used herein, includes various forms of modified or altered antibodies, such as an intact immunoglobulin, an Fv fragment containing only the light and heavy chain variable regions, an Fv fragment linked by a disulfide bond (Brinkmann et al. (1993) Proc. Natl. Acad. Sci. USA, 90: 547-551), an Fab or (Fab)′2 fragment containing the variable regions and parts of the constant regions, a single-chain antibody and the like (Bird et al. (1988) Science 242: 424-426; Huston et al. (1988) Proc. Nat. Acad. Sci. USA 85: 5879-5883). The antibody may be of animal (especially mouse or rat) or human origin or may be chimeric (Morrison et al. (1984) Proc Nat. Acad. Sci. USA 81: 6851-6855) or humanized (Jones et al. (1986) Nature 321: 522-525, and published UK patent application #8707252).

The terms “binding partner”, or “capture agent”, or a member of a “binding pair” refers to molecules that specifically bind other molecules to form a binding complex such as antibody-antigen, lectin-carbohydrate, nucleic acid-nucleic acid, biotin-avidin, etc.

The term “specifically binds”, as used herein, when referring to a biomolecule (e.g., protein, nucleic acid, antibody, etc.), refers to a binding reaction which is determinative of the presence biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 141 9), phosphorothioate (Mag et al. (1991) Nucleic Acids Res. 19:1437; and U.S. Pat. No. 5,644,048), phosphorodithioate (Briu et al. (1989) J. Am. Chem. Soc. 111 :2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences. Stringent hybridization and stringent hybridization wash conditions in the context of nucleic acid hybridization are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part 1, chapt 2, Overview of principles of hybridization and the strategy of nucleic acid probe assays, Elsevier, N.Y. (Tijssen). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook (1989) Molecular Cloning: A Laboratory Manual (2nd ed.) Vol. 1-3, Cold Spring Harbor Laboratory, Cold Spring Harbor Press, NY, and detailed discussion, below), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook supra.) for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term database refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

### BRIEF DESCRIPTION OF THE DRAWINGS

FIG. 1A shows a Venn diagram for unrestricted analysis of 88 DLPFC RNA samples showing the overlap between the number of differentially expressed genes in schizophrenia (left circle-627 genes) and bipolar disorder (right circle-1166 genes). The intersection of two circles produces a set of 327 genes. FIG. 1B shows the results of a similar analysis conducted on a restricted set of samples (brain pH >6.57) that yielded 280 genes that were shared between both disorders. FIG. 1C shows the overlap of both analyses (Venn Diagrams A and B intersections) showing differentially expressed genes (n=78) that are robust to pH differences and shared between schizophrenia and bipolar disorder.

FIGS. 2A and 2B shows the distribution of gene expression for two candidate genes (SLC1A2 (FIG. 2A), AGXT2L1 (FIG. 2B) for bipolar disorder and schizophrenia. The distribution shows restriction almost exclusively to brain regions. On a whole, brain shows almost 10 times the median expression level found in any other tissues or cell lines. The figures are from Novartis website and data was previously published [Su, 2004 #741].

FIG. 3 shows the distribution of gene expression values for AGXT2L1 for schizophrenia (top red circles-A), controls (middle blue circle-B), and bipolar disorder (bottom green circle-C). The distribution shows a bimodal distribution in AGXT2L1 values for the 88 samples combined. This further suggests that individuals with a high AGXT2L1 value are at a higher risk of developing a psychiatric disorder. Indivdiduals with psychiatric disorder (48) showed above the median control AGXT2L1 expression levels. The distribution of controls versus psychiatric disorder was highly significant for the (Fisher's Exact Test, p=0.000001), an odds ratio of 11.4 for developing a psychiatric disorder based upon above median expression of AGXT2L1.

FIG. 4. The most significant functional category (p=3.19×10⁻¹⁹) was

Cellular Growth and Proliferation that contained the following genes: BUB1B, EMX2, ERBB2, FGF2, FTH1, IL2RA, LGALS3, MAFG, NFATC1, PVR, RERG, SMCY, SMO, SOX9, TXNIP. Genes from two categories Nervous System Development and Function (labeled 1-9), and Cell Death (labeled 10) were subsets of Cellular Growth and Proliferation.

### DETAILED DESCRIPTION

### I. Diagnostic/Prognostic Methods.

This invention pertains to the discovery of biomarkers that are strong indicators for the presence of and/or a predilection to a psychiatric disorder. In particular, genes are identified herein whose expression is altered (e.g., upregulated or downregulated) in bipolar disorder and/or schizophrenia. Measurements of the expression level(s) of one, or a plurality, of these genes provides an indicator of a person having, or at elevated risk (as compared to the normal healthy population), for a psychiatric disorder. In various embodiments this indicator can be used as a component in a differential diagnosis for a person having or at risk for the disease. Moreover, the use of such indicators can inform the selection/design of a prophylactic or therapeutic treatment regimen.

Accordingly, this invention provides biomarkers that can be used by physicians to rapidly identify patient having or at risk for a psychiatric disorder, and which psychiatric patients would likely be either a schizophrenia patient or bipolar disorder patient. This permits more rapid and accurate treatment of psychiatric patients at first contact. Failure to adequately treat psychiatric patients such as bipolar disorder patients has been associated with a high risk of death by suicide.

Schizophrenia and bipolar disorder are regarded as complex disorders indicating that they are not caused by a single gene or gene expression product. The complex interplay among genes in pathways that are coordinately controlled likely confers risk to either or both disorders. These new findings provided herein, indicate that screening multiple sets and combinations of the disclosed genes will more accurately allow diagnosis, prognosis, and differential diagnosis of these disorders. Without being bound to a particular theory, it is believed that no single gene will likely cause either disorder (bipolar disorder or schizophrenia), and that many, e.g., 10s or 100s of genes and/or particular combinations/patterns of gene expression account for the large variance in pathophysiological mechanism underlying these disorders.

In various embodiments, this invention identifies patterns of gene expression, especially blood and/or brain/neurological gene expression that differentiates bipolar and schizophrenia subjects and/or that predispose a subject to either/or both illnesses. Thus, for example, in one illustrative embodiment, a blood sample can be obtained from a patient, the RNA evaluated for gene expression, and then it can be determined if a particular patient shows an expression pattern indicative of bipolar disorder or schizophrenia. Thus, for example, the expression pattern of bipolar disorder specific genes concordant in brain and lymphocytes (e.g., ATP6V1D, GSR, SH3GLB1), and/or the expression pattern of schizophrenia specific genes that are concordant in brain and lymphocytes (e.g., PPP1R3C, CYP4F11, SCEL, and the like) can be used as a diagnostic/prognostic of the disease state and/or to differentiate bipolar disorder from schizophrenia.

As shown in Example 2, blood samples from a third psychiatric group, Klinefelter syndrome, was evaluated and it was shown that these subjects did not show differences in biomarkers that either bipolar or schizophrenia groups possess.

As shown in Example 1, in certain embodiments genes are identified whose expression is altered in both schizophrenia and bipolar disorder (see, e.g., Table 6). These genes provide robust diagnostic and/or prognostic indicators for a psychiatric disorder. Thus, in certain embodiments, this invention contemplates screening a patient for one or more of these genes (upregulated or downregulated as indicated in Table 6) as a diagnostic indicator for the presence of a psychiatric disorder, or as a prognostic indicator for predisposition to a psychiatric disorder (e.g., schizophrenia and/or bipolar disorder), in, e.g., high-risk individuals from families with a psychiatric history.

Genes are also identified whose expression is substantially dysregulated in schizophrenia, but show little or no dysregulation in bipolar disorder (see, e.g., Tables 1 and 10). Similarly, genes are whose expression is substantially dysregulated in bipolar disorder, but show little or no dysregulation in schizophrenia (see, e.g., Tables 2 and 9). Measurement of the expression of these genes (Tables 1 and 10, and/or Tables 2 and 9) can be used, e.g., as a component of a differential diagnosis, to distinguish between schizophrenia and bipolar disorder. This is a particularly difficult diagnosis to make in very young children.

In addition, expression levels of one or more of the genes shown in Table 1 and/or Table 2 as well as Table 6 can be used as a diagnostic and/or prognostic for a psychiatric disorder.

Table 1 shows a list of genes whose expression is dysregulated (e.g. upregulated or downregulated) in schizophrenia and relatively unaltered in bipolar disorder. A “fold change” greater than 1 indicates upregulation (increased expression) of the gene, while a “fold change” less than 1 indicates downregulation (decreased expression) of the gene. The median expression in column 3, is whether the gene is expressed above the median level (positive number) or below the median level (negative number). In this case, 0 is no change.

| | | | | | | p- | | Fold |
|---|---|---|---|---|---|---|---|---|
| | CodeLink | Median | | | p-value | value | Fold | Change |
| Gene | Array Probe | Gene | NCBI | NCBI | Schizo- | Bipolar | Change | Bipolar |
| Symbol | Name | Expression | NID | Accession | phrenia | Disorder | Schizophrenia | Disorder |
| S100A8 | NM_002964.2_PROBE1 | −2.47 | 21614543 | NM_002964.3 | 0.0049 | 0.4648 | 4.40 | 1.46 |
| SERPINA3 | NM_001085.2_PROBE1 | −0.79 | 50659079 | NM_001085.3 | 0.0444 | 0.2473 | 2.90 | 1.87 |
| S100A9 | 3402182CB1_PROBE1 | −0.40 | 9845520 | NM_002965.2 | 0.0262 | 0.7647 | 2.57 | 1.14 |
| NULL | AK027091_PROBE1 | −0.91 | 20359307 | BQ183757.1 | 0.0006 | 0.5847 | 2.36 | 1.14 |
| TIMP4 | NM_003256.1_PROBE1 | −2.57 | 48255910 | NM_003256.2 | 0.0003 | 0.0598 | 1.78 | 1.34 |
| PP2135 | 333872.4_PROBE1 | −1.21 | 10440411 | AK024449.1 | 0.0333 | 0.1102 | 1.61 | 1.44 |
| NQO1 | NM_000903.1_PROBE1 | −1.38 | 4505414 | NM_000903.1 | 0.0153 | 0.9329 | 1.57 | 0.98 |
| FLJ21924 | NM_024774.1_PROBE1 | 3.07 | 10438134 | AK025577.1 | 0.0078 | 0.0869 | 1.54 | 1.32 |
| ADORA2B | NM_000676.1_PROBE1 | −2.00 | 22907046 | NM_000676.2 | 0.0193 | 0.0887 | 1.54 | 1.38 |
| CABLES1 | AK025627_PROBE1 | −0.01 | 24308407 | NM_138375.1 | 0.0100 | 0.0915 | 1.52 | 1.32 |
| DDX11 | NM_004399.1_PROBE1 | −0.95 | 21536327 | NM_030653.2 | 0.0474 | 0.7544 | 1.51 | 1.07 |
| FLJ10769 | 198242.3_PROBE1 | 2.40 | 8922653 | NM_018210.1 | 0.0176 | 0.8084 | 1.50 | 0.96 |
| DHRS3 | NM_004753.1_PROBE1 | 1.40 | 34222303 | NM_004753.3 | 0.0063 | 0.0502 | 1.50 | 1.34 |
| C20orf58 | 445214.7_PROBE1 | 0.03 | 42476063 | NM_152864.2 | 0.0165 | 0.2312 | 1.47 | 1.22 |
| IFITM1 | NM_003641.1_PROBE1 | 3.33 | 40254449 | NM_003641.2 | 0.0170 | 0.0887 | 1.47 | 1.32 |
| PANX2 | NM_052839.1_PROBE1 | 1.82 | 39995065 | NM_052839.2 | 0.0367 | 0.2297 | 1.46 | 1.25 |
| LRCH3 | AL137527_PROBE1 | 1.11 | 34536291 | AK128758.1 | 0.0426 | 0.7935 | 1.44 | 1.05 |
| PRSS11 | 1787335CB1_PROBE1 | 4.98 | 21327712 | NM_002775.2 | 0.0479 | 0.0512 | 1.43 | 1.44 |
| DHTKD1 | NM_018706.1_PROBE1 | −0.73 | 846162 | R72130.1 | 0.0078 | 0.1714 | 1.43 | 1.20 |
| MT1E | 1320248.3_PROBE1 | 0.83 | 13466679 | BG505162.1 | 0.0239 | 0.0599 | 1.42 | 1.35 |
| SMTN | NM_006932.2_PROBE1 | −0.11 | 19913395 | NM_134269.1 | 0.0295 | 0.0509 | 1.36 | 1.34 |
| EMP2 | NM_001424.1_PROBE1 | 0.08 | 42716292 | NM_001424.3 | 0.0352 | 0.3788 | 1.36 | 1.14 |
| ALDH7A1 | NM_001182.1_PROBE1 | 0.22 | 4557342 | NM_001182.1 | 0.0161 | 0.1476 | 1.36 | 1.20 |
| ACAA2 | NM_006111.1_PROBE1 | 0.04 | 5174428 | NM_006111.1 | 0.0470 | 0.4171 | 1.35 | 1.13 |
| ZNF256 | NM_005773.1_PROBE1 | −1.67 | 37574602 | NM_005773.2 | 0.0244 | 0.6321 | 1.35 | 1.07 |
| FBXO30 | NM_032145.2_PROBE1 | −1.05 | 54112383 | NM_032145.4 | 0.0221 | 0.0590 | 1.35 | 1.29 |
| AMID | NM_032797.1_PROBE1 | −0.90 | 31563505 | NM_032797.4 | 0.0272 | 0.6606 | 1.35 | 1.06 |
| PHKA1 | NM_002637.1_PROBE1 | −2.20 | 4505778 | NM_002637.1 | 0.0463 | 0.2883 | 1.35 | 1.18 |
| PTTG1IP | NM_004339.2_PROBE1 | 1.55 | 11038670 | NM_004339.2 | 0.0051 | 0.1144 | 1.34 | 1.18 |
| DTNA | NM_032981.1_PROBE1 | 1.96 | 42717996 | NM_032979.2 | 0.0197 | 0.4214 | 1.34 | 1.11 |
| IGFBP7 | NM_001553.1_PROBE1 | 3.20 | 4504618 | NM_001553.1 | 0.0308 | 0.1856 | 1.33 | 1.20 |
| GSTP1 | NM_000852.2_PROBE1 | 2.64 | 6552334 | NM_000852.2 | 0.0327 | 0.0543 | 1.33 | 1.31 |
| SNTA1 | NM_003098.1_PROBE1 | 2.03 | 18765742 | NM_003098.2 | 0.0148 | 0.2720 | 1.32 | 1.13 |
| ASXL1 | BF000474_PROBE1 | −1.88 | 19007752 | BM694494.1 | 0.0401 | 0.8025 | 1.31 | 0.97 |
| MYT1 | 378436.15_PROBE1 | −1.23 | 41352713 | NM_004535.2 | 0.0073 | 0.8439 | 1.31 | 1.02 |
| PCF11 | AB020631_PROBE1 | 0.79 | 33620744 | NM_015885.2 | 0.0183 | 0.1280 | 1.30 | 1.19 |
| ZNF84 | NM_003428.1_PROBE1 | −0.71 | 4508036 | NM_003428.1 | 0.0306 | 0.4175 | 1.29 | 1.10 |
| SRI | AL117616_PROBE1 | 2.15 | 38679886 | NM_003130.2 | 0.0142 | 0.1165 | 1.29 | 1.18 |
| SLC3A2 | AB018010_PROBE1 | 3.05 | 10438143 | AK025584.1 | 0.0333 | 0.1727 | 1.29 | 1.18 |
| NULL | AL049957_PROBE1 | 2.44 | 19739342 | BQ014441.1 | 0.0234 | 0.0586 | 1.28 | 1.23 |
| NME3 | NM_002513.1_PROBE1 | −1.93 | 37693992 | NM_002513.2 | 0.0395 | 0.2555 | 1.25 | 1.13 |
| GSTM4 | BF208461_PROBE1 | −0.51 | 23065556 | NM_147148.1 | 0.0122 | 0.1917 | 1.24 | 1.12 |
| WIPI49 | NM_017983.1_PROBE1 | −0.08 | 37059790 | NM_017983.3 | 0.0311 | 0.1411 | 1.23 | 1.16 |
| SSR3 | NM_007107.1_PROBE1 | 1.23 | 28416942 | NM_007107.2 | 0.0279 | 0.4993 | 1.23 | 1.07 |
| ANKRD6 | NM_014942.1_PROBE1 | 0.84 | 4589557 | AB023174.1 | 0.0410 | 0.3773 | 1.23 | 1.10 |
| KIF27 | 236658.1_PROBE2 | 0.34 | 30025500 | AY237537.1 | 0.0356 | 0.1033 | 1.23 | 1.18 |
| GCN1L1 | D86973_PROBE1 | 0.69 | 54607052 | NM_006836.1 | 0.0411 | 0.1982 | 1.20 | 1.13 |
| FAM49A | AK001942_PROBE1 | 2.06 | 16550040 | AK055334.1 | 0.0239 | 0.2788 | 0.84 | 0.92 |
| PDZK7 | NM_024895.1_PROBE1 | 0.31 | 21914924 | NM_024895.3 | 0.0441 | 0.2154 | 0.82 | 0.88 |
| OLFM1 | BC008763_PROBE1 | 4.47 | 34335281 | NM_014279.2 | 0.0362 | 0.6918 | 0.82 | 1.04 |
| ATP6V0D1 | BC008861_PROBE1 | 3.94 | 34335257 | NM_004691.3 | 0.0323 | 0.0538 | 0.81 | 0.82 |
| DOK5 | NM_018431.1_PROBE1 | 0.13 | 29544739 | NM_177959.1 | 0.0061 | 0.1751 | 0.81 | 0.90 |
| SNX25 | NM_031953.1_PROBE1 | 0.02 | 38708168 | NM_031953.2 | 0.0303 | 0.2421 | 0.81 | 0.89 |
| TOLLIP | NM_019009.1_PROBE1 | 2.55 | 966382 | D62608.1 | 0.0166 | 0.1679 | 0.80 | 0.88 |
| BCL11A | NM_022893.1_PROBE1 | 0.69 | 20336306 | NM_018014.2 | 0.0241 | 0.0799 | 0.79 | 0.83 |
| SEPT6 | NM_015129.1_PROBE1 | 1.21 | 33624799 | NM_145800.2 | 0.0355 | 0.1986 | 0.79 | 0.86 |
| C6orf149 | 1499473.2_PROBE1 | −0.20 | 38570053 | NM_020408.3 | 0.0218 | 0.6273 | 0.78 | 0.95 |
| AF1Q | NM_006818.1_PROBE1 | 3.16 | 55774979 | NM_006818.3 | 0.0260 | 0.2866 | 0.78 | 0.88 |
| FMNL1 | NM_005892.1_PROBE1 | −0.10 | 33356147 | NM_005892.3 | 0.0260 | 0.0938 | 0.77 | 0.81 |
| FLJ13842 | 230891.1_PROBE1 | 0.72 | 13375886 | NM_024645.1 | 0.0403 | 0.1175 | 0.76 | 0.81 |
| DLGAP1 | NM_004746.1_PROBE1 | −0.81 | 51339030 | NM_001003809.1 | 0.0213 | 0.2559 | 0.76 | 0.87 |
| TUBA1 | 1500780.4_PROBE1 | 2.02 | 16549334 | AK054731.1 | 0.0240 | 0.3096 | 0.75 | 0.88 |
| NULL | 1097098.1_PROBE1 | −2.07 | 23138638 | BC037807.1 | 0.0093 | 0.3862 | 0.74 | 0.91 |
| SERTAD4 | AL035414_PROBE1 | 0.13 | 1164092 | N40495.1 | 0.0080 | 0.1699 | 0.74 | 0.86 |
| PVALB | X63070_PROBE1 | 2.90 | 55925656 | NM_002854.2 | 0.0420 | 0.8888 | 0.74 | 0.98 |
| NULL | AF054994_PROBE1 | −1.73 | 3005709 | AF054994.1 | 0.0167 | 0.3424 | 0.74 | 0.89 |
| RGS4 | 617517CB1_PROBE1 | 2.67 | 38201693 | NM_005613.3 | 0.0243 | 0.0819 | 0.73 | 0.78 |
| COL15A1 | NM_001855.1_PROBE1 | −1.45 | 18641349 | NM_001855.2 | 0.0376 | 0.0861 | 0.73 | 0.76 |
| LBH | 201820.1_PROBE1 | 0.19 | 13569871 | NM_030915.1 | 0.0287 | 0.0808 | 0.72 | 0.76 |
| KCNS3 | NM_002252.1_PROBE1 | −1.84 | 25952107 | NM_002252.3 | 0.0072 | 0.8827 | 0.71 | 0.98 |
| OSBPL10 | NM_017784.1_PROBE1 | −0.81 | 23111057 | NM_017784.3 | 0.0359 | 0.0704 | 0.70 | 0.73 |
| ETV5 | NM_004454.1_PROBE1 | 1.84 | 4758315 | NM_004454.1 | 0.0203 | 0.0576 | 0.70 | 0.74 |
| SLC38A5 | 441855.8_PROBE1 | −0.81 | 15723369 | NM_033518.1 | 0.0299 | 0.4540 | 0.66 | 0.86 |
| TRPV2 | NM_016113.1_PROBE1 | −1.23 | 22547178 | NM_016113.3 | 0.0402 | 0.0615 | 0.65 | 0.67 |
| C1orf34 | 1453241CB1_PROBE1 | −2.18 | 34191891 | BC028374.2 | 0.0239 | 0.1266 | 0.65 | 0.74 |
| TAC1 | NM_013998.1_PROBE1 | 0.79 | 7770076 | NM_013997.1 | 0.0229 | 0.1580 | 0.64 | 0.76 |
| NTN4 | NM_021229.1_PROBE1 | −1.40 | 24475651 | NM_021229.2 | 0.0398 | 0.0538 | 0.61 | 0.62 |
| CRHBP | NM_001882.2_PROBE1 | −2.37 | 47080098 | NM_001882.3 | 0.0190 | 0.0557 | 0.57 | 0.60 |
| GPR161 | NM_007369.1_PROBE1 | 1.68 | 24476015 | NM_153832.1 | 0.0251 | 0.4652 | 0.54 | 0.82 |
| NELL1 | NM_006157.1_PROBE1 | −0.68 | 45269146 | NM_006157.2 | 0.0096 | 0.1551 | 0.51 | 0.68 |
| ITIH2 | NM_002216.1_PROBE1 | −1.83 | 4504782 | NM_002216.1 | 0.0001 | 0.0521 | 0.44 | 0.68 |
| ZZEF1 | NM_015113.1_PROBE1 | 1.29 | 34527911 | AK122719.1 | 0.0416 | 0.7315 | 0.43 | 0.87 |
| I-4 | NM_025210.1_PROBE1 | −5.36 | 13376811 | NM_025210.1 | 0.0049 | 0.9007 | 3.86 | 1.11 |
| IRAK3 | 960065CB1_PROBE1 | −5.24 | 6005791 | NM_007199.1 | 0.0084 | 0.0654 | 3.04 | 2.24 |
| GP2 | 2069886CB1_PROBE1 | −5.40 | 55953080 | NM_001007241.1 | 0.0220 | 0.5296 | 2.97 | 1.38 |
| MME | NM_007289.1_PROBE1 | −4.88 | 6042201 | NM_007288.1 | 0.0487 | 0.2391 | 2.79 | 2.03 |
| GRAP2 | NM_004810.1_PROBE1 | −5.11 | 19913386 | NM_004810.2 | 0.0048 | 0.0915 | 2.74 | 1.87 |
| CYP3A5 | NM_000777.2_PROBE1 | −5.53 | 15147331 | NM_000777.2 | 0.0070 | 0.1679 | 2.57 | 1.72 |
| SULT2A1 | NM_003167.1_PROBE1 | −5.33 | 29540544 | NM_003167.2 | 0.0275 | 0.1144 | 2.43 | 1.81 |
| DCDC2 | NM_016356.1_PROBE1 | −4.68 | 7706690 | NM_016356.1 | 0.0428 | 0.8691 | 2.41 | 1.08 |
| FLJ25006 | 5547067CB1_PROBE1 | −4.30 | 21389410 | NM_144610.1 | 0.0129 | 0.5434 | 2.04 | 1.20 |
| IL13 | NM_002188.1_PROBE1 | −5.45 | 26787977 | NM_002188.2 | 0.0473 | 0.2319 | 2.00 | 1.74 |
| HS3ST3B1 | NM_006042.1_PROBE2 | −4.33 | 5174466 | NM_006041.1 | 0.0198 | 0.7498 | 1.88 | 0.93 |
| GPLD1 | NM_001503.1_PROBE1 | −4.94 | 29171716 | NM_001503.2 | 0.0075 | 0.2943 | 1.84 | 1.27 |
| MEGF11 | NM_032445.1_PROBE1 | −3.87 | 14192940 | NM_032445.1 | 0.0090 | 0.4456 | 1.82 | 1.19 |
| ANTXR1 | AK002160_PROBE1 | −3.02 | 16933552 | NM_018153.2 | 0.0394 | 0.3274 | 1.55 | 1.26 |
| NULL | 127359.1_PROBE1 | −3.52 | 998645 | S78429.1 | 0.0211 | 0.6672 | 0.51 | 0.88 |
| NULL | 1354196.1_PROBE1 | −3.65 | 10437431 | AK024999.1 | 0.0091 | 0.2178 | 0.41 | 0.64 |
| NULL | AL049974_PROBE1 | −3.55 | 4884224 | AL049974.1 | 0.0264 | 0.5250 | 0.41 | 0.74 |
| DKFZP566M114 | NM_032128.1_PROBE1 | −4.80 | 52856443 | NM_032128.2 | 0.0183 | 0.0754 | 0.39 | 0.40 |
| NULL | 1397507.1_PROBE1 | −3.66 | 6407557 | AB009076.1 | 0.0267 | 0.4162 | 0.36 | 0.69 |
| C9orf13 | NM_024500.1_PROBE1 | −3.12 | 34527780 | AK122605.1 | 0.0227 | 0.1395 | 0.36 | 0.51 |
| PRO2949 | AV719512_PROBE1 | −4.67 | 28148017 | CB161891.1 | 0.0353 | 0.6011 | 0.34 | 0.77 |
| NULL | 117292.1_PROBE1 | −3.95 | | INCYTE | 0.0384 | 0.1476 | 0.34 | 0.46 |
| | | | | UNIQUE | | | | |
| PIGR | NM_002644.1_PROBE1 | −4.28 | 456345 | X73079.1 | 0.0120 | 0.2004 | 0.33 | 0.57 |
| NULL | 1465984.1_PROBE1 | −4.36 | | INCYTE | 0.0024 | 0.4263 | 0.32 | 0.75 |
| | | | | UNIQUE | | | | |
| NULL | 1503659.3_PROBE1 | −4.27 | 47939866 | BC072410.1 | 0.0426 | 0.4397 | 0.32 | 0.63 |
| SLC39A7 | NM_006979.1_PROBE1 | −3.93 | 5901935 | NM_006979.1 | 0.0000 | 0.1712 | 0.26 | 0.73 |

Table 2 shows a list of genes whose expression is dysregulated (e.g. up-regulated or down-regulated) in bipolar disorder and relatively unaltered in schizophrenia. The “Fold Change” indicates the altered regulation (expression) of the gene. A “fold change” greater than 1 indicates upregulation (increased expression) of the gene, while a “fold change” less than 1 indicates downregulation (decreased expression) of the gene. The median expression in column 3, is whether the gene is expressed above the median level (positive number) or below the median level (negative number). In that case, 0 is no change

| | | | | | | p- | | Fold |
|---|---|---|---|---|---|---|---|---|
| | CodeLink | Median | | | p-value | value | Fold | Change |
| | Array Probe | Gene | | NCBI | Schiz- | Bipolar | Change | Bipolar |
| Gene Symbol | Name | Expression | NCBI NID | Accession | ophrenia | Disorder | Schizophrenia | Disorder |
| GSTM1 | NM_000561.1_PROBE1 | 1.96 | 23065543 | NM_000561.2 | 0.20898 | 0.00007 | 1.21 | 2.02 |
| RAB25 | 903216.5_PROBE1 | −1.98 | 1382437 | W71996.1 | 0.24810 | 0.00206 | 1.22 | 1.82 |
| MDK | 1681813CB1_PROBE1 | −0.71 | 24475622 | NM_002391.2 | 0.19820 | 0.00089 | 1.21 | 1.75 |
| C10orf11 | NM_032024.1_PROBE1 | 0.54 | 24475719 | NM_032024.2 | 0.71574 | 0.02922 | 1.09 | 1.71 |
| GGTA1 | 198728.2_PROBE1 | 0.04 | 1219484 | N67359.1 | 0.11411 | 0.00145 | 1.27 | 1.69 |
| NULL | AA904423_PROBE1 | −0.90 | | INCYTE | 0.33753 | 0.00166 | 1.15 | 1.69 |
| | | | | UNIQUE | | | | |
| LOC286437 | 1500058.1_PROBE1 | 1.02 | 20364822 | BQ189271.1 | 0.28169 | 0.00063 | 1.15 | 1.68 |
| TCF7 | NM_003202.1_PROBE1 | −2.10 | 47419937 | NM_213648.1 | 0.17334 | 0.00111 | 1.21 | 1.67 |
| DKFZP586H2123 | AK027841_PROBE1 | −0.41 | 50659099 | NM_001001991.1 | 0.20345 | 0.02474 | 1.30 | 1.65 |
| NULL | 206983.1_PROBE1 | 1.29 | 31874689 | BX538220.1 | 0.14215 | 0.00147 | 1.23 | 1.65 |
| SLC1A2 | AL157452_PROBE1 | 5.56 | 40254477 | NM_004171.2 | 0.18842 | 0.02691 | 1.31 | 1.63 |
| WNT5B | NM_032642.1_PROBE1 | 0.19 | 17402918 | NM_030775.2 | 0.21210 | 0.00177 | 1.19 | 1.61 |
| ITGA7 | NM_002206.1_PROBE1 | 2.67 | 4504752 | NM_002206.1 | 0.09423 | 0.00465 | 1.29 | 1.59 |
| NULL | 330977.1_PROBE1 | −1.36 | 23290235 | BU624020.1 | 0.18692 | 0.00050 | 1.17 | 1.59 |
| FLJ32421 | 216331.1_PROBE1 | 2.05 | 21758083 | AK098136.1 | 0.14377 | 0.00183 | 1.21 | 1.56 |
| NULL | M36707_PROBE1 | −2.60 | 6441943 | AW175906.1 | 0.24877 | 0.01169 | 1.18 | 1.54 |
| NULL | 238289.3_PROBE1 | 0.33 | 3483494 | AF086149.1 | 0.16613 | 0.00561 | 1.22 | 1.53 |
| KIAA0515 | NM_003327.1_PROBE1 | 3.86 | 34366999 | BX647842.1 | 0.12769 | 0.00015 | 1.16 | 1.53 |
| PHACTR2 | NM_014721.1_PROBE1 | −1.51 | 7662247 | NM_014721.1 | 0.09301 | 0.00113 | 1.22 | 1.53 |
| GA17 | 1449840.8_PROBE1 | −0.51 | 11328545 | BF366520.1 | 0.40287 | 0.00600 | 1.12 | 1.53 |
| NUDT7 | 240037.10_PROBE1 | 1.11 | 1384548 | W73998.1 | 0.30093 | 0.01033 | 1.17 | 1.52 |
| MCC | 1445835.2_PROBE1 | 1.38 | 31873998 | BX537952.1 | 0.05886 | 0.00357 | 1.28 | 1.52 |
| ARG99 | 232773.2_PROBE1 | −0.85 | 20361940 | BQ186389.1 | 0.86395 | 0.01277 | 0.97 | 1.51 |
| NULL | BG572195_PROBE1 | −0.63 | 23647622 | BU727096.1 | 0.50937 | 0.00406 | 1.09 | 1.50 |
| FLJ10803 | 1452781.1_PROBE1 | −2.53 | 10437269 | AK024861.1 | 0.71323 | 0.00805 | 1.05 | 1.48 |
| BTN2A2 | NM_006995.2_PROBE1 | −1.92 | 31881700 | NM_006995.3 | 0.32287 | 0.00637 | 1.14 | 1.48 |
| NAG18 | 1074000.1_PROBE1 | 0.33 | 27840469 | BX117251.1 | 0.16792 | 0.00063 | 1.15 | 1.47 |
| FLJ23221 | NM_024579.1_PROBE1 | 1.33 | 13375757 | NM_024579.1 | 0.49507 | 0.02239 | 1.11 | 1.47 |
| UNC119 | NM_005148.1_PROBE1 | 0.01 | 16936534 | NM_054035.1 | 0.08714 | 0.01152 | 1.27 | 1.45 |
| NULL | 229100.1_PROBE1 | 0.44 | 4394352 | AI493349.1 | 0.2334 | 0.00021 | 1.11 | 1.45 |
| FLJ22639 | NM_024796.1_PROBE1 | −2.66 | 13376167 | NM_024796.1 | 0.30640 | 0.00998 | 1.14 | 1.45 |
| AK3 | 1384002.1_PROBE1 | 3.01 | 10439954 | AK026966.1 | 0.08560 | 0.00576 | 1.24 | 1.44 |
| FDXR | NM_024417.1_PROBE1 | 1.52 | 13435351 | NM_004110.2 | 0.32471 | 0.00774 | 1.13 | 1.44 |
| OLIG2 | AF221520_PROBE1 | −0.71 | 17978474 | NM_005806.1 | 0.31508 | 0.04744 | 1.19 | 1.44 |
| NULL | 1500926.2_PROBE1 | −0.93 | 6026929 | AW071931.1 | 0.14695 | 0.01201 | 1.21 | 1.44 |
| C8orf1 | NM_004337.1_PROBE1 | 1.99 | 4757889 | NM_004337.1 | 0.17116 | 0.00250 | 1.16 | 1.43 |
| SLC35F2 | NM_017515.1_PROBE1 | −0.94 | 34222328 | NM_017515.3 | 0.21660 | 0.00213 | 1.14 | 1.43 |
| MYH15 | 7497630CB1_PROBE1 | −1.63 | 27529749 | AB023217.2 | 0.14729 | 0.01103 | 1.21 | 1.43 |
| GOLPH3L | 978408.1_PROBE1 | 1.39 | 29826327 | NM_018178.3 | 0.31073 | 0.00256 | 1.11 | 1.43 |
| 15E1.2 | 1358135.1_PROBE1 | 1.95 | 23271010 | BC034962.1 | 0.38381 | 0.04830 | 1.16 | 1.43 |
| NEUROD1 | NM_002500.1_PROBE1 | −0.92 | 4505376 | NM_002500.1 | 0.71747 | 0.03940 | 1.06 | 1.42 |
| C15orf19 | BC007697_PROBE1 | 0.96 | 23308688 | NM_152260.1 | 0.12769 | 0.00167 | 1.17 | 1.42 |
| CRSP2 | NM_004229.1_PROBE1 | −0.88 | 28558972 | NM_004229.2 | 0.51605 | 0.00334 | 1.07 | 1.42 |
| NULL | 208876.1_PROBE1 | −0.51 | 20364957 | BQ189406.1 | 0.54525 | 0.04101 | 1.10 | 1.41 |
| FLJ11724 | AK021786_PROBE1 | −2.64 | 10433041 | AK021786.1 | 0.06601 | 0.04978 | 1.35 | 1.40 |
| RAE1 | 247605.1_PROBE1 | 0.95 | 23828470 | BU783875.1 | 0.29577 | 0.00479 | 1.12 | 1.39 |
| C7orf32 | NM_000987.1_PROBE1 | 4.91 | 50593527 | NM_145230.1 | 0.16469 | 0.01197 | 1.18 | 1.39 |
| HPS4 | 235970.15_PROBE1 | 2.35 | 23110969 | NM_152841.1 | 0.59168 | 0.01511 | 1.07 | 1.38 |
| MAP2K3 | N53854_PROBE1 | 0.97 | 21752780 | AK093838.1 | 0.15107 | 0.01320 | 1.19 | 1.38 |
| ZNF232 | NM_014519.1_PROBE1 | −0.17 | 37574600 | NM_014519.2 | 0.13200 | 0.00882 | 1.18 | 1.38 |
| ZNF606 | 1090304.1_PROBE1 | 0.53 | 566685 | Z40942.1 | 0.11613 | 0.01543 | 1.21 | 1.37 |
| DBI | 1215972CB1_PROBE1 | 5.47 | 54262129 | NM_020548.4 | 0.44436 | 0.00883 | 1.09 | 1.37 |
| OSR1 | NM_005109.1_PROBE1 | 1.66 | 4826877 | NM_005109.1 | 0.20255 | 0.00412 | 1.14 | 1.37 |
| C9orf39 | 222921.1_PROBE1 | −1.83 | 8923250 | NM_017738.1 | 0.70820 | 0.00161 | 1.03 | 1.37 |
| PPP2R5C | 1499325.2_PROBE1 | −0.31 | 1189218 | N48052.1 | 0.08176 | 0.01578 | 1.23 | 1.36 |
| MLLT3 | NM_004529.1_PROBE1 | 1.45 | 4758719 | NM_004529.1 | 0.06221 | 0.00021 | 1.15 | 1.36 |
| ABI1 | AF006516_PROBE1 | 0.30 | 34533435 | AK126803.1 | 0.10696 | 0.00684 | 1.19 | 1.36 |
| EPHB1 | 350231.3_PROBE1 | 0.33 | 55770893 | NM_004441.3 | 0.32512 | 0.00782 | 1.11 | 1.36 |
| E2-230K | AK000926_PROBE1 | 3.56 | 4820889 | F35263.1 | 0.09962 | 0.00818 | 1.19 | 1.36 |
| NSBP1 | 1442069CB1_PROBE1 | 0.05 | 13540522 | NM_030763.1 | 0.16852 | 0.00707 | 1.15 | 1.35 |
| C20orf140 | AF116909_PROBE1 | 0.93 | 21389446 | NM_144628.1 | 0.20993 | 0.00274 | 1.12 | 1.35 |
| TAF3 | 1511497.4_PROBE1 | −0.74 | 750026 | R00290.1 | 0.10776 | 0.01148 | 1.19 | 1.35 |
| NUP43 | NM_024647.1_PROBE1 | 2.22 | 38605731 | NM_024647.4 | 0.21765 | 0.02759 | 1.17 | 1.35 |
| CDCA4 | NM_017955.1_PROBE1 | −1.11 | 22027510 | NM_145701.1 | 0.10951 | 0.01710 | 1.21 | 1.35 |
| NULL | AB007900_PROBE1 | 2.48 | 19008293 | BM695035.1 | 0.07703 | 0.00660 | 1.19 | 1.35 |
| MANBA | NM_005908.1_PROBE1 | 0.38 | 24797157 | NM_005908.2 | 0.26444 | 0.02546 | 1.15 | 1.35 |
| MARS2 | 237404.1_PROBE1 | 0.29 | 56550036 | NM_138395.2 | 0.20800 | 0.00320 | 1.12 | 1.34 |
| KIAA1970 | 365379.4_PROBE1 | 0.93 | 21733057 | AL832489.1 | 0.07228 | 0.01307 | 1.22 | 1.34 |
| GNA11 | NM_002067.1_PROBE1 | 1.56 | 4504036 | NM_002067.1 | 0.08130 | 0.00956 | 1.20 | 1.33 |
| COX15 | NM_004376.1_PROBE1 | 0.28 | 37655159 | NM_078470.2 | 0.29025 | 0.02656 | 1.14 | 1.33 |
| ZNF20 | AK023094_PROBE1 | −1.66 | 33667024 | NM_021143.1 | 0.48235 | 0.01504 | 1.08 | 1.33 |
| NULL | 350126.1_PROBE1 | 0.58 | 875053 | H10231.1 | 0.12100 | 0.03265 | 1.22 | 1.33 |
| SLC26A11 | 1501505.9_PROBE1 | 0.57 | 27734747 | NM_173626.1 | 0.18988 | 0.00033 | 1.10 | 1.33 |
| ANP32E | AA772097_PROBE1 | 0.17 | 23463320 | NM_030920.2 | 0.16063 | 0.02901 | 1.18 | 1.32 |
| CHD4 | NM_001273.1_PROBE1 | 2.49 | 51599155 | NM_001273.2 | 0.22803 | 0.00853 | 1.12 | 1.31 |
| GTF2E1 | NM_005513.1_PROBE1 | 0.19 | 5031726 | NM_005513.1 | 0.13702 | 0.00543 | 1.14 | 1.31 |
| MED9 | 244363.1_PROBE1 | 0.43 | 22907057 | NM_018019.2 | 0.09102 | 0.00227 | 1.14 | 1.31 |
| KIAA1573 | 332404.3_PROBE1 | −0.66 | 32698741 | NM_020925.1 | 0.25161 | 0.00489 | 1.10 | 1.30 |
| C12orf2 | AL122049_PROBE1 | 1.26 | 55669469 | AY665468.1 | 0.19024 | 0.01117 | 1.13 | 1.30 |
| KIAA0650 | AB014550_PROBE1 | 1.26 | 3327113 | AB014550.1 | 0.11692 | 0.00735 | 1.15 | 1.30 |
| ETFDH | NM_004453.1_PROBE1 | 1.18 | 4758311 | NM_004453.1 | 0.21247 | 0.02159 | 1.14 | 1.30 |
| SLC41A1 | 1452354.6_PROBE1 | 1.40 | 51702205 | NM_173854.4 | 0.13623 | 0.00397 | 1.13 | 1.29 |
| PP35 | NM_007016.1_PROBE1 | −0.24 | 31742495 | NM_181581.1 | 0.40512 | 0.03349 | 1.10 | 1.29 |
| LOC221362 | BC003416_PROBE1 | −1.54 | 21749410 | AK091117.1 | 0.31108 | 0.04106 | 1.12 | 1.29 |
| USP24 | AB028980_PROBE1 | 1.01 | 21732933 | AL832370.1 | 0.12791 | 0.01481 | 1.16 | 1.28 |
| NIPBL | W81491_PROBE1 | 1.09 | 47578104 | NM_133433.2 | 0.17323 | 0.03435 | 1.16 | 1.28 |
| LOC132241 | AL117606_PROBE1 | 0.82 | 21756119 | AK096589.1 | 0.23043 | 0.02804 | 1.13 | 1.28 |
| FKSG24 | 236006.6_PROBE1 | 0.30 | 14249259 | NM_032683.1 | 0.25893 | 0.01669 | 1.11 | 1.28 |
| AS3MT | AA429850_PROBE1 | −0.38 | 24476003 | NM_020682.2 | 0.11164 | 0.00631 | 1.14 | 1.28 |
| LRP1B | NM_018557.1_PROBE1 | 0.89 | 9055269 | NM_018557.1 | 0.42151 | 0.04563 | 1.10 | 1.27 |
| CXorf39 | 251178.1_PROBE1 | 2.97 | 1068426 | H86847.1 | 0.26197 | 0.00398 | 1.09 | 1.27 |
| PIGH | NM_004569.1_PROBE1 | 1.92 | 24430187 | NM_004569.2 | 0.37132 | 0.03287 | 1.10 | 1.26 |
| PGAP1 | 241020.1_PROBE1 | 1.65 | 27930371 | CB104564.1 | 0.60778 | 0.04268 | 1.06 | 1.26 |
| POLR3G | 980742CB1_PROBE1 | −2.29 | 5454017 | NM_006467.1 | 0.11735 | 0.00590 | 1.13 | 1.26 |
| TGFBRAP1 | NM_030825.1_PROBE1 | 2.10 | 15997861 | BI857114.1 | 0.76781 | 0.02727 | 1.03 | 1.26 |
| ENAH | NM_018212.1_PROBE1 | 2.11 | 50345275 | NM_018212.3 | 0.30285 | 0.03539 | 1.11 | 1.26 |
| RAB18 | BC015014_PROBE1 | 4.00 | 34222129 | NM_021252.3 | 0.18558 | 0.00661 | 1.11 | 1.25 |
| NULL | 345354.2_PROBE1 | 1.52 | 18044363 | BC020243.1 | 0.20361 | 0.00287 | 1.09 | 1.25 |
| ATAD2 | NM_014109.1_PROBE1 | −0.54 | 24497617 | NM_014109.2 | 0.90219 | 0.04559 | 0.99 | 1.25 |
| RAB22A | 202347.1_PROBE1 | 3.35 | 34577103 | NM_020673.2 | 0.85445 | 0.01463 | 0.98 | 1.25 |
| LOC284611 | 237667.2_PROBE1 | 0.97 | 10437667 | AK025203.1 | 0.88306 | 0.04307 | 1.02 | 1.25 |
| LOC123722 | 401923.10_PROBE1 | 1.84 | 21733929 | AL833295.1 | 0.26426 | 0.02864 | 1.11 | 1.25 |
| ZNF236 | NM_007345.1_PROBE1 | −0.28 | 10092585 | NM_007345.1 | 0.60436 | 0.01009 | 1.04 | 1.24 |
| ANKRD27 | NM_032139.1_PROBE1 | 2.43 | 14149802 | NM_032139.1 | 0.36228 | 0.03219 | 1.09 | 1.24 |
| C9orf12 | NM_022755.1_PROBE1 | −1.82 | 14091076 | AF351201.1 | 0.15661 | 0.04363 | 1.15 | 1.23 |
| RABL3 | AK025772_PROBE1 | 0.77 | 34366750 | BX647593.1 | 0.10360 | 0.02922 | 1.16 | 1.23 |
| LNX2 | 120682.1_PROBE1 | 0.64 | 34222215 | NM_153371.2 | 0.42779 | 0.02390 | 1.07 | 1.23 |
| KIAA1648 | U88870_PROBE1 | −0.87 | 21750910 | AK092338.1 | 0.77318 | 0.03077 | 1.03 | 1.23 |
| PAICS | NM_006452.1_PROBE1 | 1.16 | 17388802 | NM_006452.2 | 0.46924 | 0.00422 | 1.05 | 1.23 |
| C15orf25 | 1448211.1_PROBE1 | −1.50 | 10434618 | AK022939.1 | 0.68604 | 0.02890 | 1.04 | 1.23 |
| ZBTB34 | 197784.1_PROBE1 | 1.10 | 21693131 | AB082524.1 | 0.48309 | 0.04772 | 1.07 | 1.23 |
| RBBP9 | 196590.2_PROBE1 | −0.07 | 24119167 | NM_153328.1 | 0.41416 | 0.02905 | 1.07 | 1.23 |
| ZNF418 | 2686104CB1_PROBE1 | −1.77 | 52138522 | NM_133460.1 | 0.94869 | 0.03935 | 0.99 | 1.22 |
| SGPL1 | AB033078_PROBE1 | −0.16 | 31982935 | NM_003901.2 | 0.12377 | 0.04659 | 1.16 | 1.22 |
| EEF1A1 | 4528729CB1_PROBE1 | 3.95 | 12384837 | BF982025.1 | 0.48335 | 0.01318 | 1.05 | 1.22 |
| CAD | NM_004341.1_PROBE1 | −0.96 | 47458828 | NM_004341.3 | 0.44527 | 0.02167 | 1.06 | 1.22 |
| NCOR1 | NM_006311.1_PROBE1 | 2.33 | 22538460 | NM_006311.2 | 0.27672 | 0.03839 | 1.10 | 1.21 |
| NFIA | AK024964_PROBE2 | 0.99 | 815590 | R53688.1 | 0.14642 | 0.04195 | 1.13 | 1.20 |
| MGC16824 | NM_020314.1_PROBE1 | 0.71 | 31543158 | NM_020314.3 | 0.56315 | 0.03889 | 1.05 | 1.20 |
| FLJ23451 | 336542.4_PROBE1 | −0.25 | 13376108 | NM_024766.1 | 0.16694 | 0.01914 | 1.10 | 1.19 |
| MGC23908 | 241165.14_PROBE1 | 1.06 | 14042660 | AK027750.1 | 0.48352 | 0.02210 | 0.96 | 1.17 |
| C9orf81 | 131604.6_PROBE1 | −0.34 | 8151805 | AW962072.1 | 0.19912 | 0.01867 | 1.08 | 1.16 |
| LMAN2L | NM_030805.1_PROBE1 | 0.03 | 13540593 | NM_030805.1 | 0.65563 | 0.02968 | 1.03 | 1.16 |
| TCFL4 | NM_013383.1_PROBE1 | −0.88 | 38201613 | NM_198205.1 | 0.38571 | 0.02834 | 0.95 | 0.86 |
| SCAMP1 | AK001541_PROBE1 | 3.57 | 33598918 | NM_052822.2 | 0.67318 | 0.04400 | 0.97 | 0.85 |
| BZW1 | NM_014670.1_PROBE1 | 2.92 | 41281428 | NM_014670.2 | 0.83919 | 0.02224 | 1.01 | 0.85 |
| CDCA8 | NM_018101.1_PROBE1 | 0.32 | 51593099 | NM_018101.2 | 0.28634 | 0.04787 | 0.91 | 0.83 |
| SMOC1 | NM_022137.1_PROBE1 | −0.08 | 51871617 | NM_022137.3 | 0.72206 | 0.04114 | 0.97 | 0.83 |
| NDST1 | U18932_PROBE1 | −0.19 | 46094001 | NM_001543.3 | 0.71977 | 0.01336 | 0.97 | 0.82 |
| TAF6L | NM_006473.1_PROBE1 | −1.03 | 21269867 | NM_006473.2 | 0.33960 | 0.03952 | 0.92 | 0.82 |
| XCL1 | NM_002995.1_PROBE1 | −0.89 | 4506852 | NM_002995.1 | 0.50873 | 0.03674 | 0.94 | 0.82 |
| PSMB5 | 2496996CB1_PROBE1 | 3.20 | 22538468 | NM_002797.2 | 0.07305 | 0.01326 | 0.87 | 0.82 |
| ZNF263 | NM_005741.1_PROBE1 | −0.16 | 34222305 | NM_005741.3 | 0.82403 | 0.03191 | 0.98 | 0.81 |
| VLDLR | NM_003383.1_PROBE1 | 0.00 | 40254472 | NM_003383.2 | 0.80279 | 0.03093 | 0.98 | 0.81 |
| DBC1 | NM_014618.1_PROBE1 | 3.10 | 7657008 | NM_014618.1 | 0.30863 | 0.00897 | 0.93 | 0.81 |
| ILT10 | NM_024317.1_PROBE1 | −0.04 | 28866948 | NM_024317.2 | 0.32030 | 0.02935 | 0.91 | 0.81 |
| MGC2747 | NM_024104.1_PROBE1 | 2.51 | 34147357 | NM_024104.2 | 0.31986 | 0.00368 | 0.94 | 0.81 |
| KIAA1549 | 337771.6_PROBE1 | −0.94 | 876146 | H11326.1 | 0.05459 | 0.04902 | 0.82 | 0.81 |
| PTCH | 107346.1_PROBE1 | 0.65 | 6698986 | AW292350.1 | 0.79481 | 0.02187 | 0.98 | 0.81 |
| GEMIN4 | 2014478CB1_PROBE1 | −0.58 | 7657121 | NM_015721.1 | 0.26412 | 0.04761 | 0.89 | 0.81 |
| KIAA0368 | AB002366_PROBE1 | 0.16 | 34530414 | AK124590.1 | 0.75393 | 0.02210 | 0.97 | 0.81 |
| NDUFS6 | 3120581CB1_PROBE1 | 3.99 | 39812335 | NM_004553.2 | 0.14544 | 0.04371 | 0.86 | 0.80 |
| AK1 | NM_000476.1_PROBE1 | 1.66 | 4502010 | NM_000476.1 | 0.32244 | 0.00777 | 0.93 | 0.80 |
| COMMD4 | NM_017828.1_PROBE1 | 1.90 | 34530908 | AK124968.1 | 0.42864 | 0.04527 | 0.92 | 0.80 |
| BMS1L | NM_014753.1_PROBE1 | −0.41 | 41281482 | NM_014753.2 | 0.72173 | 0.02119 | 0.97 | 0.80 |
| SCO1 | NM_004589.1_PROBE1 | 0.95 | 4759067 | NM_004589.1 | 0.84160 | 0.02661 | 0.98 | 0.80 |
| MGC15523 | 1502671.13_PROBE1 | −0.96 | 17123883 | BM129331.1 | 0.24403 | 0.04714 | 0.88 | 0.80 |
| AUP1 | NM_012103.1_PROBE1 | 1.37 | 32313582 | NM_012103.2 | 0.60528 | 0.03170 | 0.95 | 0.80 |
| VPS13D | NM_018156.1_PROBE1 | −0.90 | 1623432 | AA080880.1 | 0.42528 | 0.01871 | 0.93 | 0.79 |
| ETS2 | 405457.26_PROBE1 | 1.46 | 56119171 | NM_005239.4 | 0.11233 | 0.01301 | 0.87 | 0.79 |
| C11orf17 | NM_020642.1_PROBE1 | 1.02 | 33667100 | NM_182901.1 | 0.20458 | 0.00785 | 0.90 | 0.79 |
| FLJ20232 | NM_019008.1_PROBE1 | −0.74 | 42766427 | NM_019008.4 | 0.68614 | 0.03070 | 0.96 | 0.79 |
| EIF2S1 | NM_004094.1_PROBE1 | 0.70 | 34147492 | NM_004094.3 | 0.55577 | 0.01124 | 1.05 | 0.79 |
| NULL | AA481297_PROBE1 | 0.92 | | INCYTE | 0.41285 | 0.00604 | 0.94 | 0.78 |
| | | | | UNIQUE | | | | |
| PFKL | 1386636.1_PROBE1 | −0.34 | 5433244 | AL045070.1 | 0.40921 | 0.00166 | 0.94 | 0.78 |
| MPPE1 | NM_023075.1_PROBE1 | −1.50 | 41281674 | NM_138608.1 | 0.06165 | 0.02892 | 0.82 | 0.78 |
| FAHD1 | 198617.2_PROBE1 | 1.81 | 13654273 | NM_031208.1 | 0.82846 | 0.04283 | 0.98 | 0.78 |
| C5orf14 | NM_024715.1_PROBE1 | −1.49 | 21362011 | NM_024715.2 | 0.61935 | 0.02615 | 0.95 | 0.78 |
| STAC | NM_003149.1_PROBE1 | −0.78 | 4507246 | NM_003149.1 | 0.50613 | 0.03613 | 0.93 | 0.78 |
| GSPT2 | NM_018094.1_PROBE1 | 0.29 | 46094013 | NM_018094.2 | 0.34470 | 0.02629 | 0.91 | 0.78 |
| PPP1R16B | 1448277.1_PROBE1 | 0.68 | 10162778 | BE748786.1 | 0.26484 | 0.03727 | 0.88 | 0.78 |
| RBMS1 | 70305380CB1_PROBE1 | 1.79 | 46249389 | NM_016836.2 | 0.70616 | 0.00976 | 1.03 | 0.78 |
| ATP5J2 | NM_004889.1_PROBE1 | 3.93 | 51479131 | NM_001003714.1 | 0.84269 | 0.03899 | 0.98 | 0.78 |
| MGC17337 | 253996.2_PROBE1 | −1.62 | 1686889 | AA127601.1 | 0.23704 | 0.03459 | 0.87 | 0.77 |
| COMMD6 | 1382376.1_PROBE1 | 3.01 | 45333908 | NM_203497.1 | 0.96558 | 0.04703 | 0.99 | 0.77 |
| NULL | 1104111.1_PROBE1 | −0.28 | | INCYTE | 0.18574 | 0.02746 | 0.87 | 0.77 |
| | | | | UNIQUE | | | | |
| POLE4 | AAF46843_PROBE1 | 0.61 | 38455393 | NM_019896.2 | 0.97900 | 0.03815 | 1.00 | 0.77 |
| ELK4 | NM_021795.1_PROBE1 | 0.97 | 41872461 | NM_021795.2 | 0.64897 | 0.03764 | 0.95 | 0.77 |
| MRPL33 | NM_004891.1_PROBE1 | 4.46 | 21735608 | NM_145330.1 | 0.35206 | 0.01650 | 0.91 | 0.77 |
| PTP4A3 | NM_032611.1_PROBE1 | −1.42 | 14589853 | NM_007079.2 | 0.89938 | 0.03110 | 1.01 | 0.77 |
| NULL | 1334866.1_PROBE1 | −1.40 | | INCYTE | 0.97965 | 0.01741 | 1.00 | 0.77 |
| | | | | UNIQUE | | | | |
| HDAC7A | NM_016596.2_PROBE1 | −0.01 | 10436761 | AK024387.1 | 0.74049 | 0.03774 | 1.04 | 0.77 |
| LOC150763 | 1439946.1_PROBE1 | 0.35 | 15929580 | BC015216.1 | 0.16288 | 0.01008 | 0.88 | 0.77 |
| B2M | 1725857CB1_PROBE1 | 3.84 | 37704380 | NM_004048.2 | 0.58397 | 0.00295 | 0.96 | 0.77 |
| SRP14 | 242477.1_PROBE1 | 0.13 | 15934899 | BI823349.1 | 0.64536 | 0.02628 | 1.05 | 0.77 |
| SSBP1 | NM_003143.1_PROBE1 | 2.13 | 4507230 | NM_003143.1 | 0.41827 | 0.02776 | 0.91 | 0.76 |
| NULL | 1452853.11_PROBE1 | −0.98 | | INCYTE | 0.68219 | 0.01522 | 0.96 | 0.76 |
| | | | | UNIQUE | | | | |
| RGS4 | 232915.1_PROBE1 | 3.70 | 38201693 | NM_005613.3 | 0.40851 | 0.03913 | 0.90 | 0.76 |
| CNNM2 | NM_017649.1_PROBE1 | 0.04 | 40068052 | NM_017649.3 | 0.32769 | 0.01696 | 0.90 | 0.76 |
| HPCAL1 | 6032576CB1_PROBE1 | 1.02 | 19913442 | NM_134421.1 | 0.41334 | 0.00992 | 0.92 | 0.76 |
| PP3111 | NM_022156.1_PROBE1 | 0.65 | 40807365 | NM_022156.3 | 0.39725 | 0.00267 | 0.93 | 0.76 |
| LOC286144 | 475186.4_PROBE2 | 1.10 | 11493402 | AF130048.1 | 0.91515 | 0.04183 | 0.99 | 0.76 |
| CXCL1 | NM_001511.1_PROBE1 | −1.08 | 4504152 | NM_001511.1 | 0.44342 | 0.04787 | 0.90 | 0.76 |
| DPM1 | NM_003859.1_PROBE1 | 2.21 | 4503362 | NM_003859.1 | 0.26463 | 0.01143 | 0.89 | 0.76 |
| C6orf15 | NM_014070.1_PROBE1 | −0.74 | 7662666 | NM_014070.1 | 0.37889 | 0.03258 | 0.90 | 0.75 |
| NULL | 977815.8_PROBE1 | 2.36 | | INCYTE | 0.10239 | 0.02837 | 0.82 | 0.75 |
| | | | | UNIQUE | | | | |
| IL1R2 | NM_004633.1_PROBE1 | −0.68 | 27894333 | NM_173343.1 | 0.79538 | 0.01321 | 1.03 | 0.75 |
| MRPL28 | NM_006428.1_PROBE1 | 1.41 | 39812062 | NM_006428.3 | 0.08665 | 0.03120 | 0.81 | 0.75 |
| RPL35 | 786379CB1_PROBE1 | 1.87 | 16117792 | NM_007209.2 | 0.84358 | 0.01026 | 0.98 | 0.75 |
| ISCU | 1544462CB1_PROBE1 | 1.97 | 56699455 | NM_213595.1 | 0.80265 | 0.02545 | 0.97 | 0.75 |
| C21orf33 | NM_004649.1_PROBE1 | 1.82 | 38026959 | NM_198155.1 | 0.67047 | 0.00494 | 0.96 | 0.75 |
| MRGPRD | 1330308.1_PROBE1 | −0.66 | 42794264 | NM_198923.2 | 0.23393 | 0.03270 | 0.86 | 0.75 |
| RDBP | 1998002CB1_PROBE1 | 2.73 | 20631983 | NM_002904.4 | 0.58375 | 0.00608 | 0.95 | 0.75 |
| SECISBP2 | 1501774.11_PROBE1 | 1.62 | 21359954 | NM_024077.2 | 0.87497 | 0.01704 | 0.98 | 0.75 |
| CNOT10 | NM_015442.1_PROBE1 | −0.82 | 13123771 | NM_015442.1 | 0.87292 | 0.01363 | 0.98 | 0.75 |
| ANKS1 | D86982_PROBE1 | 1.59 | 38683796 | NM_015245.1 | 0.47468 | 0.02630 | 0.92 | 0.75 |
| FTH1 | NM_002032.1_PROBE1 | 6.12 | 56682958 | NM_002032.2 | 0.27334 | 0.00227 | 1.10 | 0.75 |
| GABARAPL2 | NM_007285.2_PROBE1 | 4.60 | 27374999 | NM_007285.6 | 0.76607 | 0.00409 | 0.97 | 0.75 |
| C9orf78 | NM_016482.1_PROBE1 | −0.92 | 24475983 | NM_016482.2 | 0.58664 | 0.00257 | 0.95 | 0.75 |
| DNAJB11 | NM_016306.1_PROBE1 | 1.43 | 51317390 | NM_016306.4 | 0.65293 | 0.00502 | 0.96 | 0.74 |
| MRPL12 | NM_002949.1_PROBE1 | 1.74 | 27436900 | NM_002949.2 | 0.51290 | 0.00287 | 1.06 | 0.74 |
| IER5 | NM_016545.1_PROBE1 | 2.28 | 45439368 | NM_016545.3 | 0.74098 | 0.03908 | 1.04 | 0.74 |
| VMD2L1 | NM_017682.1_PROBE1 | 0.26 | 8923136 | NM_017682.1 | 0.34978 | 0.00077 | 0.93 | 0.74 |
| DCHS1 | NM_024542.1_PROBE1 | 1.20 | 16933556 | NM_003737.1 | 0.27226 | 0.03461 | 0.86 | 0.74 |
| C11orf24 | NM_022338.1_PROBE1 | 1.61 | 52851412 | NM_022338.2 | 0.81782 | 0.00067 | 1.02 | 0.74 |
| NULL | 111821.1_PROBE1 | 1.78 | | INCYTE | 0.79870 | 0.00165 | 0.98 | 0.74 |
| | | | | UNIQUE | | | | |
| NDUFB1 | NM_004545.1_PROBE1 | 3.82 | 38569472 | NM_004545.3 | 0.54181 | 0.03682 | 0.92 | 0.74 |
| NULL | 1003649.1_PROBE1 | −0.26 | | INCYTE | 0.35560 | 0.00123 | 0.93 | 0.74 |
| | | | | UNIQUE | | | | |
| PSMB9 | NM_002800.1_PROBE1 | −0.13 | 23110931 | NM_148954.1 | 0.92111 | 0.04638 | 1.01 | 0.74 |
| e(y)2 | NM_020189.1_PROBE1 | 0.66 | 34222364 | NM_020189.4 | 0.42962 | 0.02024 | 0.91 | 0.74 |
| IL10RB | 1452813.8_PROBE1 | −0.48 | 24430214 | NM_000628.3 | 0.78804 | 0.04847 | 0.96 | 0.74 |
| TMSB10 | 3993708CB1_PROBE1 | 4.42 | 31543813 | NM_021103.2 | 0.86945 | 0.02080 | 0.98 | 0.73 |
| USP14 | NM_005151.1_PROBE1 | 2.10 | 20070184 | NM_005151.2 | 0.15374 | 0.00030 | 0.90 | 0.73 |
| TBCA | NM_004607.1_PROBE1 | 3.27 | 4759211 | NM_004607.1 | 0.26833 | 0.00147 | 0.91 | 0.73 |
| UQCRFS1 | NM_006003.1_PROBE1 | 3.84 | 5174742 | NM_006003.1 | 0.37855 | 0.00415 | 0.92 | 0.73 |
| IL4R | NM_000418.1_PROBE1 | −1.65 | 56788409 | NM_000418.2 | 0.87585 | 0.01366 | 1.02 | 0.73 |
| OMG | NM_002544.1_PROBE1 | 3.31 | 52426786 | NM_002544.3 | 0.97341 | 0.04094 | 1.00 | 0.73 |
| CREM | NM_001881.1_PROBE1 | 0.36 | 34335225 | NM_183013.1 | 0.25774 | 0.00698 | 0.89 | 0.73 |
| VARS2L | AB067472_PROBE1 | −1.19 | 55741844 | NM_020442.3 | 0.19101 | 0.03800 | 0.83 | 0.73 |
| ATRX | NM_000489.1_PROBE1 | 1.58 | 33354052 | AB102641.1 | 0.92346 | 0.02337 | 0.99 | 0.73 |
| LSM8 | 3964476CB1_PROBE1 | −0.47 | 21314665 | NM_016200.2 | 0.70871 | 0.01789 | 0.96 | 0.73 |
| NULL | 1505215.1_PROBE1 | 0.58 | 2657017 | AA676495.1 | 0.69965 | 0.00916 | 0.96 | 0.73 |
| EB1 | NM_020140.1_PROBE1 | 0.78 | 50511944 | NM_152788.3 | 0.76111 | 0.02133 | 1.04 | 0.73 |
| NPDC1 | NM_015392.1_PROBE1 | 2.82 | 20149616 | NM_015392.2 | 0.84584 | 0.01032 | 1.02 | 0.73 |
| EEF2 | NM_001961.1_PROBE1 | 4.28 | 25453476 | NM_001961.2 | 0.34928 | 0.03108 | 1.14 | 0.73 |
| MRPL20 | NM_017971.1_PROBE1 | 1.57 | 26638656 | NM_017971.2 | 0.56791 | 0.04876 | 0.92 | 0.73 |
| NDUFB6 | NM_002493.1_PROBE1 | 1.52 | 33519470 | NM_002493.3 | 0.23192 | 0.00271 | 0.89 | 0.73 |
| TBC1D7 | AF151073_PROBE1 | 1.60 | 24475975 | NM_016495.2 | 0.63489 | 0.01483 | 0.94 | 0.73 |
| FLJ14668 | NM_032822.1_PROBE1 | −1.04 | 14249519 | NM_032822.1 | 0.19145 | 0.01457 | 0.85 | 0.73 |
| MGC61716 | AK025374_PROBE1 | 0.37 | 42740902 | NM_182501.2 | 0.94472 | 0.02089 | 1.01 | 0.73 |
| AKAP12 | NM_005100.1_PROBE1 | 2.13 | 21493021 | NM_005100.2 | 0.40809 | 0.03016 | 1.12 | 0.72 |
| RAB1A | 045200CB1_PROBE1 | 1.05 | 41350195 | NM_004161.3 | 0.42496 | 0.00744 | 0.92 | 0.72 |
| TDE1 | AA406107_PROBE1 | 2.74 | 39812087 | NM_006811.2 | 0.83784 | 0.00901 | 0.98 | 0.72 |
| MK-STYX | NM_016086.1_PROBE1 | −0.78 | 32481212 | NM_016086.2 | 0.97332 | 0.00420 | 1.00 | 0.72 |
| PDCD5 | NM_004708.1_PROBE1 | −0.18 | 21735599 | NM_004708.2 | 0.77444 | 0.00945 | 0.97 | 0.72 |
| TUBA6 | NM_032704.1_PROBE1 | 0.77 | 31880337 | NM_032704.2 | 0.14464 | 0.03261 | 0.81 | 0.72 |
| SON | AL163262_PROBE1 | −0.13 | 21040313 | NM_032195.1 | 0.71585 | 0.04881 | 0.95 | 0.72 |
| VDR | NM_000376.1_PROBE1 | 0.19 | 4507882 | NM_000376.1 | 0.06901 | 0.00011 | 0.87 | 0.72 |
| TCEAL4 | NM_024863.1_PROBE1 | 2.57 | 55749458 | NM_001006937.1 | 0.39292 | 0.01393 | 0.90 | 0.72 |
| LGALS12 | NM_033101.1_PROBE1 | −0.81 | 20127658 | NM_033101.2 | 0.32809 | 0.03345 | 0.87 | 0.72 |
| NDUFA5 | NM_005000.2_PROBE1 | 3.58 | 13699821 | NM_005000.2 | 0.91982 | 0.02194 | 0.99 | 0.72 |
| MRPL11 | NM_016050.1_PROBE1 | −1.12 | 25306271 | NM_170738.1 | 0.18013 | 0.01315 | 0.84 | 0.71 |
| MFN1 | NM_017927.1_PROBE1 | 0.06 | 45269136 | NM_033540.2 | 0.53703 | 0.01517 | 0.92 | 0.71 |
| COPB | 114971.3_PROBE1 | −0.76 | 34536417 | AK128844.1 | 0.30729 | 0.00681 | 0.89 | 0.71 |
| RPL21 | 1327135.17_PROBE1 | −0.34 | 1580256 | C18654.1 | 0.22917 | 0.00112 | 0.89 | 0.71 |
| PCDHB14 | NM_018934.2_PROBE1 | 0.17 | 14195602 | NM_018934.2 | 0.13310 | 0.00561 | 0.84 | 0.71 |
| MGC4606 | NM_024516.1_PROBE1 | −1.08 | 39725648 | NM_024516.2 | 0.98687 | 0.03456 | 1.00 | 0.71 |
| HSPA4 | AB023420_PROBE1 | 1.55 | 38327038 | NM_002154.3 | 0.25019 | 0.02655 | 1.18 | 0.71 |
| MGC13168 | NM_032735.1_PROBE1 | 0.62 | 14249355 | NM_032735.1 | 0.25156 | 0.00113 | 0.90 | 0.71 |
| RANBP5 | 1453567.1_PROBE1 | −0.18 | 10031043 | BE670502.1 | 0.16965 | 0.01452 | 0.83 | 0.71 |
| ZNF184 | 1068701.2_PROBE1 | −1.50 | 20412282 | BQ230882.1 | 0.65010 | 0.00307 | 0.95 | 0.70 |
| NDUFV1 | NM_007103.1_PROBE1 | 1.74 | 20149567 | NM_007103.2 | 0.68468 | 0.03086 | 0.94 | 0.70 |
| SEC61A2 | NM_018144.2_PROBE1 | 1.55 | 14589846 | NM_018144.2 | 0.68670 | 0.00690 | 0.95 | 0.70 |
| C14orf92 | NM_014828.1_PROBE1 | 0.46 | 7662273 | NM_014828.1 | 0.70061 | 0.00708 | 1.05 | 0.70 |
| LOC51321 | NM_016627.1_PROBE1 | 2.47 | 46195796 | NM_016627.3 | 0.93741 | 0.03317 | 1.01 | 0.70 |
| C11orf1 | NM_022761.1_PROBE1 | −1.08 | 12232430 | NM_022761.1 | 0.69674 | 0.01894 | 1.06 | 0.70 |
| HFE | NM_000410.1_PROBE1 | −2.28 | 21040342 | NM_139004.1 | 0.61848 | 0.00780 | 0.94 | 0.70 |
| B3GALT7 | 235663.7_PROBE1 | −1.97 | 42821106 | NM_198540.2 | 0.73723 | 0.02812 | 0.95 | 0.70 |
| ZDHHC7 | NM_017740.1_PROBE1 | 3.57 | 8923254 | NM_017740.1 | 0.70545 | 0.00100 | 0.96 | 0.70 |
| ATP2B1 | NM_001682.1_PROBE1 | 1.89 | 48255946 | NM_001001323.1 | 0.26800 | 0.01039 | 0.87 | 0.70 |
| MGC14288 | NM_032901.1_PROBE1 | 0.31 | 34147446 | NM_032901.2 | 0.27713 | 0.02032 | 0.85 | 0.70 |
| RBM28 | NM_018077.1_PROBE1 | −2.68 | 8922387 | NM_018077.1 | 0.66334 | 0.03593 | 1.07 | 0.69 |
| RPA3 | NM_002947.1_PROBE1 | 0.41 | 52851430 | NM_002947.3 | 0.09874 | 0.01580 | 0.79 | 0.69 |
| UBL5 | NM_024292.1_PROBE1 | 3.17 | 42476283 | NM_024292.2 | 0.95843 | 0.00479 | 0.99 | 0.69 |
| RPL37 | 3595376CB1_PROBE1 | 3.58 | 16306560 | NM_000997.2 | 0.52502 | 0.02067 | 0.91 | 0.69 |
| SNRPG | NM_003096.1_PROBE1 | 0.83 | 23652611 | BU729583.1 | 0.60620 | 0.00900 | 0.94 | 0.69 |
| TALDO1 | NM_006755.1_PROBE1 | 0.25 | 5803186 | NM_006755.1 | 0.68461 | 0.02411 | 0.94 | 0.69 |
| POLR21 | 1447766.1_PROBE1 | 0.86 | 47933390 | NM_006233.4 | 0.11742 | 0.00244 | 0.84 | 0.69 |
| FLJ20530 | NM_017864.1_PROBE1 | −1.24 | 8923495 | NM_017864.1 | 0.28107 | 0.00107 | 0.90 | 0.69 |
| XLHSRF-1 | NM_015512.1_PROBE1 | 0.25 | 55741856 | NM_015512.3 | 0.43951 | 0.00042 | 0.93 | 0.69 |
| CEBPG | NM_001806.1_PROBE1 | −0.24 | 727293 | U20240.1 | 0.77766 | 0.00803 | 0.96 | 0.69 |
| DKFZp547I094 | NM_032155.1_PROBE1 | −2.72 | 14149832 | NM_032155.1 | 0.08465 | 0.00951 | 0.79 | 0.69 |
| VDAC3 | NM_005662.1_PROBE1 | 1.69 | 25188178 | NM_005662.3 | 0.20693 | 0.01183 | 0.84 | 0.68 |
| CD47 | NM_001777.1_PROBE1 | −1.06 | 396175 | X69398.1 | 0.50720 | 0.00006 | 0.95 | 0.68 |
| SMPD3 | 481722.5_PROBE1 | 1.59 | 46358429 | NM_018667.2 | 0.34343 | 0.01163 | 0.88 | 0.68 |
| FRG1 | 1661268CB1_PROBE1 | 0.92 | 4758403 | NM_004477.1 | 0.94993 | 0.02284 | 1.01 | 0.68 |
| STAB1 | NM_015136.1_PROBE1 | −0.92 | 12225239 | NM_015136.1 | 0.08851 | 0.00567 | 0.81 | 0.68 |
| NULL | M77233_PROBE1 | −2.06 | 2368178 | AA583569.1 | 0.68863 | 0.01186 | 1.06 | 0.68 |
| MYO1B | AK000160_PROBE1 | −0.75 | 44889480 | NM_012223.2 | 0.34848 | 0.00899 | 0.88 | 0.68 |
| FKSG2 | 7488456CB1_PROBE1 | −0.45 | 11056001 | NM_021631.1 | 0.40436 | 0.00726 | 0.90 | 0.68 |
| AP4S1 | NM_007077.1_PROBE1 | −1.16 | 5689378 | AB030654.1 | 0.36463 | 0.03216 | 0.86 | 0.68 |
| WASL | 1357182.1_PROBE1 | −2.28 | 10437816 | AK025323.1 | 0.25065 | 0.01564 | 0.84 | 0.68 |
| TRIM8 | NM_030912.1_PROBE1 | 1.46 | 13569865 | NM_030912.1 | 0.60893 | 0.02620 | 0.92 | 0.68 |
| TXN | NM_003329.1_PROBE1 | 2.50 | 50592993 | NM_003329.2 | 0.32673 | 0.00299 | 0.89 | 0.68 |
| TMSB4X | NM_021109.1_PROBE1 | 5.37 | 34328943 | NM_021109.2 | 0.23139 | 0.00195 | 0.87 | 0.67 |
| MCF2 | NM_005369.1_PROBE1 | 0.06 | 19923309 | NM_005369.2 | 0.76295 | 0.02635 | 0.95 | 0.67 |
| C10orf97 | NM_024948.1_PROBE1 | 0.86 | 56676388 | NM_024948.2 | 0.09611 | 0.00334 | 0.81 | 0.67 |
| NULL | 1503311.1_PROBE1 | −1.68 | 47286445 | CN270031.1 | 0.16584 | 0.01189 | 0.82 | 0.67 |
| ORMDL1 | 901176.10_PROBE1 | −1.83 | 47392450 | CN404905.1 | 0.60277 | 0.03265 | 0.91 | 0.67 |
| MAGEL2 | NM_019066.1_PROBE1 | −0.56 | 18765721 | NM_019066.2 | 0.44442 | 0.02779 | 0.88 | 0.67 |
| FLJ21019 | NM_024927.1_PROBE1 | −0.94 | 40255046 | NM_024927.3 | 0.91029 | 0.00230 | 0.99 | 0.67 |
| ZW10 | NM_004724.1_PROBE1 | −0.13 | 17136150 | NM_004724.2 | 0.14992 | 0.00121 | 0.85 | 0.67 |
| ITGB2 | NM_000211.1_PROBE1 | −1.66 | 4557885 | NM_000211.1 | 0.08549 | 0.01199 | 0.77 | 0.67 |
| LOC9884 | NM_018001.1_PROBE1 | 1.09 | 41281502 | NM_014834.2 | 0.98494 | 0.01868 | 1.00 | 0.67 |
| GLMN | 1398763.1_PROBE1 | −0.81 | 1218153 | N66028.1 | 0.31562 | 0.03377 | 0.84 | 0.67 |
| CINP | NM_032630.1_PROBE1 | −0.85 | 21327682 | NM_032630.2 | 0.27204 | 0.02326 | 1.20 | 0.67 |
| GSR | 1508570.1_PROBE1 | −1.12 | 749279 | T99542.1 | 0.51133 | 0.01473 | 1.11 | 0.66 |
| MAF | AF055376_PROBE1 | 0.82 | 3335147 | AF055376.1 | 0.12052 | 0.00409 | 0.82 | 0.66 |
| HSPC023 | NM_014047.1_PROBE1 | 2.75 | 7661741 | NM_014047.1 | 0.53398 | 0.00237 | 0.93 | 0.66 |
| C18orf37 | 221706.6_PROBE1 | −0.03 | 42822883 | NM_194281.2 | 0.77208 | 0.01466 | 0.96 | 0.66 |
| SPATS2 | 337862.4_PROBE1 | 0.15 | 12751480 | NM_023071.1 | 0.43742 | 0.01171 | 0.89 | 0.66 |
| FUCA2 | NM_032020.1_PROBE1 | −0.54 | 40068511 | NM_032020.3 | 0.93973 | 0.00455 | 1.01 | 0.66 |
| OAZ1 | NM_004152.1_PROBE1 | 3.49 | 34486089 | NM_004152.2 | 0.73184 | 0.04522 | 1.07 | 0.66 |
| CD59 | 1504971.1_PROBE1 | 0.96 | 17965386 | BM272108.1 | 0.28521 | 0.02278 | 1.20 | 0.66 |
| HUMMLC2B | AA328929_PROBE1 | −2.26 | 52108538 | BP235628.1 | 0.23376 | 0.01612 | 0.82 | 0.66 |
| COL16A1 | NM_001856.1_PROBE1 | −1.02 | 18641351 | NM_001856.2 | 0.98618 | 0.04096 | 1.00 | 0.66 |
| NRBF2 | NM_030759.1_PROBE1 | −0.41 | 13540514 | NM_030759.1 | 0.21520 | 0.00394 | 0.85 | 0.65 |
| MRPL23 | 138642.4_PROBE1 | −2.78 | 21734108 | AL833465.1 | 0.12629 | 0.03695 | 0.74 | 0.65 |
| C10orf38 | AL050367_PROBE1 | 1.75 | 4914600 | AL050367.1 | 0.27741 | 0.00774 | 0.85 | 0.65 |
| RPL29 | 1378166CB1_PROBE1 | 2.51 | 17105395 | NM_000992.2 | 0.74791 | 0.02615 | 0.94 | 0.65 |
| ASL | NM_000048.1_PROBE1 | −0.20 | 31541963 | NM_000048.2 | 0.14077 | 0.00427 | 0.82 | 0.65 |
| NDUFA10 | 2138834CB1_PROBE1 | 1.43 | 33519462 | NM_004544.2 | 0.47259 | 0.00914 | 0.90 | 0.65 |
| HBLD2 | BC002675_PROBE1 | 0.32 | 52426767 | NM_030940.3 | 0.87112 | 0.03048 | 0.97 | 0.65 |
| KCTD12 | AF052169_PROBE1 | 1.28 | 40255011 | NM_138444.2 | 0.19007 | 0.00272 | 1.19 | 0.65 |
| NULL | 1502787.2_PROBE1 | −1.27 | | INCYTE | 0.07629 | 0.00397 | 0.78 | 0.65 |
| | | | | UNIQUE | | | | |
| ALDOA | NM_000034.1_PROBE1 | 3.70 | 34577109 | NM_184041.1 | 0.85198 | 0.00248 | 1.02 | 0.65 |
| RNF139 | NM_007218.1_PROBE1 | −1.65 | 38045935 | NM_007218.3 | 0.15560 | 0.00745 | 0.81 | 0.65 |
| KIAA1449 | AB040882_PROBE1 | −0.54 | 21314694 | NM_020839.2 | 0.65192 | 0.01261 | 1.07 | 0.65 |
| LOC158160 | NM_016371.1_PROBE1 | 0.17 | 33469144 | NM_182829.1 | 0.16404 | 0.01660 | 0.79 | 0.65 |
| ATP6V1D | NM_015994.1_PROBE1 | 2.96 | 19913437 | NM_015994.2 | 0.08134 | 0.00278 | 0.79 | 0.65 |
| JAM2 | NM_021219.1_PROBE1 | −1.45 | 21704284 | NM_021219.2 | 0.97682 | 0.00689 | 1.00 | 0.65 |
| SLC11A2 | NM_000617.1_PROBE1 | 0.56 | 10835168 | NM_000617.1 | 0.84219 | 0.00144 | 1.02 | 0.65 |
| KIAA0528 | AB011100_PROBE1 | 0.21 | 29789059 | NM_014802.1 | 0.37011 | 0.00916 | 0.87 | 0.64 |
| KLK12 | NM_019598.1_PROBE1 | −0.65 | 22208988 | NM_145895.1 | 0.44455 | 0.00847 | 0.89 | 0.64 |
| FKSG17 | NM_032031.1_PROBE1 | −0.89 | 12276119 | AF315951.1 | 0.20129 | 0.01102 | 0.82 | 0.64 |
| POLE4 | NM_019896.1_PROBE1 | 0.86 | 38455393 | NM_019896.2 | 0.99120 | 0.00255 | 1.00 | 0.64 |
| TOMM70A | NM_014820.1_PROBE1 | 2.05 | 54607134 | NM_014820.3 | 0.87352 | 0.00383 | 1.02 | 0.64 |
| PSD4 | NM_012455.1_PROBE1 | −2.95 | 56788369 | NM_012455.2 | 0.31496 | 0.00610 | 0.86 | 0.64 |
| NULL | 1398859.1_PROBE1 | 0.24 | | INCYTE | 0.51258 | 0.00517 | 0.91 | 0.64 |
| | | | | UNIQUE | | | | |
| PPP1R7 | NM_002712.1_PROBE1 | 1.32 | 4506012 | NM_002712.1 | 0.97624 | 0.01532 | 1.00 | 0.64 |
| HRMT1L1 | 1382145.2_PROBE1 | −0.12 | 19727267 | BQ002367.1 | 0.71976 | 0.00184 | 1.05 | 0.64 |
| COPS2 | NM_004236.1_PROBE1 | 2.07 | 4759263 | NM_004236.1 | 0.94449 | 0.00160 | 1.01 | 0.64 |
| TERF2IP | NM_018975.1_PROBE1 | 3.94 | 52627148 | NM_018975.2 | 0.34247 | 0.00230 | 0.88 | 0.64 |
| APP | NM_000484.1_PROBE1 | 3.69 | 41406056 | NM_201414.1 | 0.48419 | 0.01380 | 0.89 | 0.64 |
| FLII | NM_002018.1_PROBE1 | 1.17 | 22547155 | NM_002018.2 | 0.09811 | 0.00220 | 0.80 | 0.64 |
| VPS28 | NM_016208.1_PROBE1 | 1.59 | 34452692 | NM_183057.1 | 0.31866 | 0.00052 | 0.89 | 0.64 |
| SET | NM_003011.1_PROBE1 | 2.12 | 4506890 | NM_003011.1 | 0.17031 | 0.00113 | 0.84 | 0.64 |
| PRPF4B | AB011108_PROBE1 | −0.03 | 28872758 | NM_176800.1 | 0.95564 | 0.02682 | 0.99 | 0.63 |
| MGC2803 | NM_024038.1_PROBE1 | 2.20 | 34147352 | NM_024038.2 | 0.28354 | 0.00039 | 0.89 | 0.63 |
| SERPINF1 | NM_002615.1_PROBE1 | 2.82 | 54792142 | NM_002615.4 | 0.17130 | 0.00066 | 0.85 | 0.63 |
| MAN2A1 | NM_002372.1_PROBE1 | −0.82 | 51477713 | NM_002372.2 | 0.66175 | 0.01744 | 0.93 | 0.63 |
| RAC1 | D25274_PROBE1 | 2.66 | 38505164 | NM_198829.1 | 0.53847 | 0.00320 | 0.92 | 0.63 |
| NULL | 7499583CB1_PROBE1 | −1.82 | 1836910 | AA077436.1 | 0.98385 | 0.03049 | 1.00 | 0.63 |
| POLR2B | NM_000938.1_PROBE1 | 1.65 | 4505940 | NM_000938.1 | 0.41977 | 0.00128 | 0.90 | 0.63 |
| BRIX | NM_018321.1_PROBE1 | −0.72 | 55770899 | NM_018321.3 | 0.47856 | 0.00148 | 1.10 | 0.63 |
| FLJ11196 | NM_018357.1_PROBE1 | 1.43 | 37537709 | NM_018357.2 | 0.36646 | 0.00475 | 0.87 | 0.63 |
| KARS | NM_005548.1_PROBE1 | 0.21 | 5031814 | NM_005548.1 | 0.86746 | 0.02131 | 1.03 | 0.63 |
| CROP | NM_016424.1_PROBE1 | 0.12 | 52426742 | NM_006107.2 | 0.98149 | 0.01990 | 1.00 | 0.63 |
| SH3GLB1 | 1990126CB1_PROBE1 | −0.47 | 21359904 | NM_016009.2 | 0.79893 | 0.00254 | 0.97 | 0.62 |
| DGCR6L | NM_005675.2_PROBE1 | 1.77 | 15718677 | NM_033257.2 | 0.55579 | 0.00231 | 1.09 | 0.62 |
| C20orf40 | NM_014054.1_PROBE1 | −1.00 | 5670252 | AF165185.1 | 0.50955 | 0.00244 | 0.91 | 0.62 |
| NXF5 | NM_032946.1_PROBE1 | −1.95 | 15487665 | NM_033154.1 | 0.32906 | 0.02468 | 0.82 | 0.62 |
| DNAJD1 | NM_013238.1_PROBE1 | 0.19 | 7019452 | NM_013238.1 | 0.81327 | 0.00363 | 1.04 | 0.62 |
| ICOSL | AB014553_PROBE1 | −2.30 | 46255054 | NM_015259.3 | 0.60441 | 0.04309 | 0.89 | 0.62 |
| ARL5 | NM_012097.1_PROBE1 | 0.60 | 29542730 | NM_177985.1 | 0.92676 | 0.01325 | 1.02 | 0.62 |
| GABARAP | NM_007278.1_PROBE1 | 2.48 | 6005763 | NM_007278.1 | 0.52382 | 0.00928 | 1.11 | 0.62 |
| PPP6C | NM_016294.1_PROBE1 | 0.09 | 20127429 | NM_002721.3 | 0.31313 | 0.00210 | 0.87 | 0.61 |
| MPHOSPH9 | AL096751_PROBE1 | −1.99 | 37537695 | NM_022782.2 | 0.45129 | 0.00408 | 0.89 | 0.61 |
| C13orf6 | NM_032859.1_PROBE1 | −0.42 | 14042767 | AK027812.1 | 0.61370 | 0.00175 | 1.07 | 0.61 |
| NULL | AA147817_PROBE1 | −2.34 | 1717251 | AA147817.1 | 0.44936 | 0.00366 | 0.89 | 0.61 |
| POLR1C | AF008442_PROBE1 | −1.52 | 42560249 | NM_004875.2 | 0.55284 | 0.00648 | 0.91 | 0.61 |
| PSMB4 | NM_002796.1_PROBE1 | 1.30 | 22538466 | NM_002796.2 | 0.23565 | 0.00564 | 0.82 | 0.61 |
| MGC12981 | NM_032357.1_PROBE1 | 0.82 | 21362049 | NM_032357.2 | 0.23412 | 0.00003 | 0.89 | 0.60 |
| SH3BGRL3 | 2870970CB1_PROBE1 | 2.10 | 42476331 | NM_031286.2 | 0.39616 | 0.00632 | 0.87 | 0.60 |
| APG3L | NM_022488.1_PROBE1 | 1.27 | 34147490 | NM_022488.3 | 0.57449 | 0.00225 | 0.92 | 0.60 |
| KRTAP19-1 | 335653.4_PROBE1 | −2.17 | 25005261 | AJ457067.1 | 0.14251 | 0.00471 | 0.79 | 0.60 |
| STX17 | AW972895_PROBE1 | −0.09 | 2265761 | AA524833.1 | 0.51399 | 0.00247 | 1.10 | 0.60 |
| NULL | 1382894.51_PROBE1 | 1.54 | 12345491 | BF978276.1 | 0.57397 | 0.00855 | 0.91 | 0.60 |
| NULL | 1501260.1_PROBE1 | −2.39 | | INCYTE | 0.12732 | 0.00251 | 0.79 | 0.60 |
| | | | | UNIQUE | | | | |
| APOA4 | NM_000482.2_PROBE1 | −2.10 | 5174773 | NM_000482.2 | 0.21806 | 0.00913 | 0.81 | 0.60 |
| HDAC4 | NM_032923.1_PROBE1 | −2.43 | 14079550 | BG768897.1 | 0.15776 | 0.01373 | 0.76 | 0.60 |
| C19orf24 | NM_017914.1_PROBE1 | −0.76 | 42476017 | NM_017914.2 | 0.51861 | 0.00508 | 1.11 | 0.60 |
| SST | NM_001048.1_PROBE1 | 3.97 | 40254432 | NM_001048.2 | 0.07231 | 0.00699 | 0.73 | 0.60 |
| SSA2 | 198318.1_PROBE1 | 1.10 | 862164 | R82773.1 | 0.74776 | 0.00059 | 1.04 | 0.60 |
| NULL | 121358.1_PROBE1 | 2.12 | | INCYTE | 0.29437 | 0.02172 | 0.79 | 0.60 |
| | | | | UNIQUE | | | | |
| AGPAT3 | NM_031487.1_PROBE1 | 1.23 | 21733505 | AL832919.1 | 0.23825 | 0.00655 | 0.81 | 0.60 |
| NULL | AA196960_PROBE1 | 4.12 | | INCYTE | 0.07077 | 0.00472 | 0.74 | 0.59 |
| | | | | UNIQUE | | | | |
| C8B | NM_000066.1_PROBE1 | −2.58 | 4557390 | NM_000066.1 | 0.77412 | 0.04322 | 0.93 | 0.59 |
| TIGD1 | 2871131CB1_PROBE1 | −1.96 | 22209000 | NM_145702.1 | 0.72310 | 0.02683 | 0.93 | 0.59 |
| LIAS | 1111994.11_PROBE1 | −0.73 | 37577165 | NM_006859.2 | 0.80727 | 0.00253 | 0.96 | 0.59 |
| KCMF1 | NM_020122.1_PROBE1 | 0.99 | 46852177 | NM_020122.3 | 0.49647 | 0.00423 | 0.89 | 0.59 |
| NULL | 1388851.1_PROBE1 | −0.30 | | INCYTE | 0.82501 | 0.00576 | 0.96 | 0.59 |
| | | | | UNIQUE | | | | |
| RPS6KA3 | 208350.1_PROBE1 | −0.48 | 56243494 | NM_004586.2 | 0.60737 | 0.01060 | 0.91 | 0.59 |
| NULL | AA702043_PROBE1 | −0.33 | | INCYTE | 0.95023 | 0.00031 | 0.99 | 0.59 |
| | | | | UNIQUE | | | | |
| APOD | 138634CB1_PROBE1 | 4.00 | 4502162 | NM_001647.1 | 0.31703 | 0.00025 | 0.88 | 0.59 |
| DUSP5 | NM_004419.2_PROBE1 | −1.60 | 12707565 | NM_004419.2 | 0.50037 | 0.02506 | 0.86 | 0.59 |
| PAM | NM_000919.1_PROBE1 | −2.05 | 21070973 | NM_138766.1 | 0.37120 | 0.00295 | 0.87 | 0.59 |
| NULL | 2817769CB1_PROBE1 | 0.79 | 10315035 | BE866155.1 | 0.22468 | 0.00461 | 0.81 | 0.59 |
| EPB41L3 | 5547766CB1_PROBE1 | 3.31 | 32490571 | NM_012307.2 | 0.05195 | 0.00084 | 0.76 | 0.59 |
| LOC51123 | 5443527CB1_PROBE1 | 0.93 | 46358346 | NM_016096.2 | 0.19209 | 0.00198 | 0.82 | 0.59 |
| KIAA1462 | AL050154_PROBE1 | −0.17 | 856410 | R80129.1 | 0.38149 | 0.00394 | 0.86 | 0.59 |
| SNF1LK | NM_030751.1_PROBE1 | 0.60 | 48762713 | NM_173354.2 | 0.20858 | 0.03525 | 0.74 | 0.59 |
| ACYP1 | 3206312CB1_PROBE1 | 0.08 | 45243546 | NM_203488.1 | 0.50419 | 0.01677 | 0.87 | 0.59 |
| RAPGEF1 | 1382593.1_PROBE1 | −1.02 | 38373674 | NM_005312.2 | 0.26069 | 0.00320 | 0.83 | 0.58 |
| NQO2 | NM_000904.1_PROBE1 | −0.55 | 4505416 | NM_000904.1 | 0.17455 | 0.00319 | 0.80 | 0.58 |
| PP3856 | 1634103CB1_PROBE1 | 0.37 | 40255088 | NM_145201.3 | 0.42091 | 0.02646 | 0.83 | 0.58 |
| EXOSC9 | NM_005033.1_PROBE1 | −0.26 | 4826921 | NM_005033.1 | 0.09963 | 0.00076 | 0.79 | 0.58 |
| LY6H | NM_002347.1_PROBE1 | −1.37 | 49574518 | NM_002347.2 | 0.93137 | 0.01746 | 0.98 | 0.58 |
| FLJ34389 | 333544.12_PROBE1 | −2.14 | 22749322 | NM_152649.1 | 0.81675 | 0.01987 | 1.05 | 0.58 |
| ARMC8 | NM_014154.1_PROBE1 | −0.75 | 6841349 | AF161541.1 | 0.35199 | 0.00691 | 0.84 | 0.58 |
| PFDN4 | NM_002623.2_PROBE1 | 0.48 | 54792079 | NM_002623.3 | 0.80632 | 0.02025 | 0.95 | 0.58 |
| SUMO2 | 1599583CB1_PROBE1 | 1.20 | 18524123 | BM475081.1 | 0.47250 | 0.00435 | 0.88 | 0.58 |
| KIAA1838 | 379018.5_PROBE1 | −0.61 | 24308333 | NM_032448.1 | 0.49332 | 0.00054 | 0.91 | 0.58 |
| UGP2 | NM_006759.2_PROBE1 | 3.10 | 48255967 | NM_001001521.1 | 0.69652 | 0.00367 | 0.94 | 0.58 |
| CLTA | 7498280CB1_PROBE1 | 2.06 | 4502898 | NM_001833.1 | 0.14310 | 0.00012 | 0.83 | 0.57 |
| ATP5A1 | NM_004046.1_PROBE1 | 3.23 | 50345981 | NM_001001935.1 | 0.26423 | 0.00260 | 0.83 | 0.57 |
| PRKAR1A | NM_002734.1_PROBE1 | 0.17 | 1526989 | M33336.1 | 0.82864 | 0.00032 | 0.97 | 0.57 |
| TLE4 | NM_007005.1_PROBE1 | 1.98 | 38327621 | NM_007005.3 | 0.32153 | 0.00024 | 0.88 | 0.57 |
| CYP11B1 | NM_000498.2_PROBE1 | −2.73 | 13904852 | NM_000497.2 | 0.87375 | 0.01344 | 0.97 | 0.57 |
| PPIA | 2899485CB1_PROBE1 | 3.70 | 45439312 | NM_203431.1 | 0.26641 | 0.00086 | 0.85 | 0.57 |
| RRP22 | NM_006477.1_PROBE1 | 1.08 | 55953119 | NM_001007279.1 | 0.55284 | 0.00687 | 0.89 | 0.57 |
| LOC158563 | AK025562_PROBE1 | −2.51 | 30353856 | BC051691.1 | 0.86509 | 0.00439 | 1.03 | 0.57 |
| HSPA1A | NM_005346.2_PROBE1 | 0.99 | 17511779 | BC018740.1 | 0.74080 | 0.01851 | 1.08 | 0.57 |
| C6orf115 | 215861.3_PROBE1 | 0.67 | 5542741 | AI868763.1 | 0.27241 | 0.00051 | 0.85 | 0.57 |
| ANAPC5 | NM_016237.1_PROBE1 | 1.72 | 34147585 | NM_016237.3 | 0.20447 | 0.00012 | 0.85 | 0.57 |
| HSGT1 | NM_007265.1_PROBE1 | −0.46 | 6005783 | NM_007265.1 | 0.89851 | 0.00045 | 1.02 | 0.57 |
| ATRN | NM_012070.1_PROBE1 | −0.58 | 21450862 | NM_139322.1 | 0.20920 | 0.00241 | 0.81 | 0.57 |
| NULL | 1462914.1_PROBE1 | −2.86 | | INCYTE | 0.71215 | 0.03033 | 0.92 | 0.57 |
| | | | | UNIQUE | | | | |
| KIAA0218 | NM_014760.1_PROBE1 | −1.66 | 7662007 | NM_014760.1 | 0.33140 | 0.01999 | 0.80 | 0.57 |
| EDD | NM_015902.3_PROBE1 | 0.46 | 41352716 | NM_015902.4 | 0.67817 | 0.00059 | 0.94 | 0.57 |
| ATP8A2 | AL390129_PROBE1 | −1.60 | 38372937 | NM_016529.3 | 0.84167 | 0.03234 | 0.95 | 0.56 |
| FTSJ3 | NM_017647.1_PROBE1 | −0.73 | 17017990 | NM_017647.2 | 0.71580 | 0.00030 | 0.95 | 0.56 |
| C7orf35 | 1861434CB1_PROBE1 | −0.63 | 24475725 | NM_032936.2 | 0.36794 | 0.00243 | 0.86 | 0.56 |
| COX6C | 2660420CB1_PROBE1 | 2.30 | 17999531 | NM_004374.2 | 0.23207 | 0.00358 | 0.81 | 0.56 |
| NULL | 1100348.1_PROBE1 | −2.24 | | INCYTE | 0.68812 | 0.01307 | 1.08 | 0.56 |
| | | | | UNIQUE | | | | |
| ATPIF1 | NM_016311.1_PROBE1 | 1.81 | 30260191 | NM_178191.1 | 0.64157 | 0.00138 | 0.93 | 0.56 |
| ARPC3 | NM_005719.1_PROBE1 | −1.56 | 23397667 | NM_005719.2 | 0.15684 | 0.00235 | 0.78 | 0.56 |
| CGI-100 | AL080084_PROBE1 | 0.11 | 47271455 | NM_016040.3 | 0.67563 | 0.01544 | 1.09 | 0.56 |
| C5orf18 | M73547_PROBE1 | −0.51 | 33667050 | NM_005669.3 | 0.86413 | 0.01879 | 0.96 | 0.56 |
| FANCL | NM_018062.1_PROBE1 | 0.28 | 49472818 | NM_018062.2 | 0.74931 | 0.00108 | 0.95 | 0.56 |
| OR6N1 | 1330314.1_1_PROBE1 | −2.73 | 52353275 | NM_001005185.1 | 0.26893 | 0.00388 | 0.82 | 0.56 |
| EHBP1 | AB020710_PROBE1 | −0.04 | 44771179 | NM_015252.2 | 0.79889 | 0.01034 | 0.95 | 0.56 |
| DF | NM_001928.1_PROBE1 | −2.48 | 42544238 | NM_001928.2 | 0.13577 | 0.02321 | 0.69 | 0.55 |
| TEBP | 1557874CB1_PROBE1 | 1.93 | 23308578 | NM_006601.4 | 0.82290 | 0.00248 | 0.96 | 0.55 |
| RBP2 | NM_004164.1_PROBE1 | −2.21 | 40354213 | NM_004164.2 | 0.15802 | 0.00479 | 0.76 | 0.55 |
| PSMD7 | NM_002811.1_PROBE1 | 1.69 | 34335279 | NM_002811.3 | 0.50091 | 0.00031 | 0.91 | 0.55 |
| LSS | 1394197.1_PROBE1 | 1.07 | 47933394 | NM_002340.3 | 0.37198 | 0.00243 | 0.85 | 0.55 |
| MAPK1 | 1445432.1_PROBE1 | 1.25 | 843326 | R69809.1 | 0.39611 | 0.02044 | 0.82 | 0.55 |
| PC4 | 002150CB1_PROBE1 | 2.53 | 19923783 | NM_006713.2 | 0.46018 | 0.00095 | 0.89 | 0.55 |
| SDCCAG10 | NM_005869.1_PROBE1 | −1.33 | 5031958 | NM_005869.1 | 0.36457 | 0.00251 | 0.85 | 0.54 |
| PITPNA | NM_006224.1_PROBE1 | −0.91 | 1060902 | D30036.1 | 0.35015 | 0.00126 | 0.85 | 0.54 |
| P8 | NM_012385.1_PROBE1 | −1.65 | 6912569 | NM_012385.1 | 0.54785 | 0.01109 | 1.14 | 0.54 |
| NULL | 1017978.1_PROBE1 | −2.49 | | INCYTE | 0.48878 | 0.00481 | 0.87 | 0.54 |
| | | | | UNIQUE | | | | |
| UBE2B | NM_003337.1_PROBE1 | −0.53 | 32967281 | NM_003337.2 | 0.50704 | 0.00423 | 0.88 | 0.54 |
| THY28 | NM_014174.1_PROBE1 | −1.91 | 40806217 | NM_014174.2 | 0.35013 | 0.01939 | 0.80 | 0.54 |
| UBE2A | NM_003336.1_PROBE1 | 1.61 | 32967279 | NM_003336.2 | 0.50243 | 0.00168 | 0.89 | 0.54 |
| ATXN10 | NM_013236.1_PROBE1 | 1.42 | 51093837 | NM_013236.2 | 0.22598 | 0.00168 | 0.81 | 0.54 |
| p44S10 | NM_014814.1_PROBE1 | 0.78 | 7661913 | NM_014814.1 | 0.40958 | 0.00742 | 0.84 | 0.54 |
| BTNL2 | 7488416CB1_PROBE1 | −2.56 | 9624968 | NM_019602.1 | 0.76132 | 0.02475 | 0.93 | 0.54 |
| NULL | N77046_PROBE1 | −0.02 | | INCYTE | 0.26037 | 0.02380 | 0.75 | 0.54 |
| | | | | UNIQUE | | | | |
| TAF11 | NM_005643.1_PROBE1 | −1.22 | 21269863 | NM_005643.2 | 0.14588 | 0.00204 | 0.77 | 0.54 |
| MYO1A | NM_005379.1_PROBE1 | −2.09 | 29544746 | NM_005379.2 | 0.29490 | 0.02341 | 0.77 | 0.54 |
| DEK | 1330593CB1_PROBE1 | 0.34 | 31542502 | NM_003472.2 | 0.28065 | 0.00080 | 0.84 | 0.53 |
| H3F3A | 4832672CB1_PROBE1 | 2.48 | 52630340 | NM_002107.3 | 0.95420 | 0.00332 | 0.99 | 0.53 |
| WIPI49 | NM_014960.1_PROBE1 | −2.80 | 11083905 | BF196206.1 | 0.99519 | 0.01588 | 1.00 | 0.53 |
| RYBP | AB029551_PROBE1 | 0.27 | 6714542 | AB029551.1 | 0.63550 | 0.00067 | 0.93 | 0.53 |
| TH1L | NM_016397.1_PROBE1 | −1.19 | 39812483 | NM_016397.2 | 0.61840 | 0.00813 | 0.90 | 0.53 |
| CHCHD6 | 198141.7_PROBE1 | −1.16 | 14150133 | NM_032343.1 | 0.13130 | 0.00060 | 0.78 | 0.53 |
| DNAJC1 | 1098496.17_PROBE1 | −2.04 | 1481851 | AA018596.1 | 0.25163 | 0.00567 | 0.79 | 0.53 |
| RY1 | X76302_PROBE1 | −0.43 | 24307918 | NM_006857.1 | 0.59469 | 0.01191 | 0.88 | 0.53 |
| PB1 | NM_018313.1_PROBE1 | −2.33 | 41281916 | NM_181042.1 | 0.91253 | 0.02801 | 1.03 | 0.53 |
| GOLGA5 | NM_005113.1_PROBE1 | −1.04 | 30260187 | NM_005113.2 | 0.70515 | 0.00361 | 0.93 | 0.53 |
| NULL | 990855.1_PROBE1 | −2.64 | | INCYTE | 0.30154 | 0.01468 | 0.78 | 0.52 |
| | | | | UNIQUE | | | | |
| NULL | 1062675.5_PROBE1 | −2.79 | 50497840 | CR617033.1 | 0.85376 | 0.00179 | 1.04 | 0.52 |
| B3GNT1 | NM_033252.1_PROBE1 | −0.30 | 15451893 | NM_006577.3 | 0.39189 | 0.00129 | 0.85 | 0.51 |
| STAT4 | NM_003151.1_PROBE1 | 0.56 | 21618332 | NM_003151.2 | 0.89488 | 0.01038 | 1.03 | 0.51 |
| PDZRN4 | NM_013377.1_PROBE1 | −2.65 | 39653318 | NM_013377.2 | 0.47307 | 0.03110 | 0.81 | 0.51 |
| RPL23 | 406573.1_PROBE1 | −2.26 | 6474027 | AW195035.1 | 0.99970 | 0.01918 | 1.00 | 0.51 |
| GDA | AB033084_PROBE1 | 1.54 | 45580724 | NM_004293.2 | 0.23321 | 0.00448 | 0.77 | 0.51 |
| NULL | 977592.1_PROBE1 | 0.20 | 667501 | T63636.1 | 0.53135 | 0.00327 | 0.88 | 0.51 |
| DNAJA1 | 2767012CB1_PROBE1 | 2.38 | 49472820 | NM_001539.2 | 0.37028 | 0.00041 | 0.86 | 0.51 |
| FAHD2A | 444677.83_PROBE1 | −1.38 | 7705607 | NM_016044.1 | 0.28567 | 0.00659 | 0.78 | 0.51 |
| C1orf37 | AL133052_PROBE1 | 1.06 | 30280447 | CB985923.1 | 0.16501 | 0.00037 | 0.79 | 0.50 |
| MYO5B | AB032945_PROBE1 | −2.08 | 29421189 | AB032945.2 | 0.11159 | 0.02629 | 0.63 | 0.50 |
| BAD | NM_032989.1_PROBE1 | 0.76 | 14670386 | NM_004322.2 | 0.76838 | 0.00437 | 0.94 | 0.50 |
| OR5V1 | NM_030876.2_PROBE1 | −2.37 | 45594309 | NM_030876.4 | 0.36672 | 0.00731 | 0.81 | 0.50 |
| NULL | 222324.1_PROBE2 | −2.14 | 19733949 | BQ009048.1 | 0.19662 | 0.00141 | 0.78 | 0.50 |
| PSMD2 | NM_002808.1_PROBE1 | 0.11 | 25777601 | NM_002808.3 | 0.23021 | 0.00148 | 0.79 | 0.50 |
| KCTD4 | 1952155CB1_PROBE1 | −0.42 | 38257143 | NM_198404.1 | 0.79326 | 0.00072 | 0.95 | 0.49 |
| NULL | 333610.5_PROBE1 | −2.09 | 1071507 | H89247.1 | 0.66929 | 0.00806 | 1.11 | 0.49 |
| IVNS1ABP | NM_016389.1_PROBE1 | −0.14 | 54144641 | NM_016389.2 | 0.22582 | 0.00007 | 0.83 | 0.49 |
| CALML4 | 1502278.11_PROBE1 | −2.31 | 1193934 | N52768.1 | 0.55320 | 0.00293 | 1.14 | 0.49 |
| AUH | NM_001698.1_PROBE1 | −1.02 | 4502326 | NM_001698.1 | 0.59513 | 0.00900 | 0.88 | 0.49 |
| SLC13A2 | NM_003984.1_PROBE1 | −2.53 | 4506978 | NM_003984.1 | 0.16656 | 0.02075 | 0.67 | 0.49 |
| PCDHB10 | NM_018930.2_PROBE1 | −0.09 | 52486036 | NM_018930.3 | 0.05944 | 0.00012 | 0.73 | 0.49 |
| KHDRBS1 | NM_006559.1_PROBE1 | 0.59 | 5730026 | NM_006559.1 | 0.49193 | 0.00096 | 0.88 | 0.49 |
| SCG2 | NM_003469.2_PROBE1 | 0.27 | 10800415 | NM_003469.2 | 0.26985 | 0.00038 | 0.82 | 0.49 |
| ZNF179 | NM_007148.1_PROBE1 | −2.97 | 23199981 | NM_007148.2 | 0.27197 | 0.02929 | 0.71 | 0.48 |
| FBXW7 | NM_018315.1_PROBE1 | −1.12 | 16117780 | NM_033632.1 | 0.98629 | 0.00385 | 1.00 | 0.48 |
| SCAP2 | NM_003930.1_PROBE1 | −0.42 | 38202227 | NM_003930.3 | 0.54003 | 0.01717 | 0.84 | 0.48 |
| HTR1E | NM_000865.1_PROBE1 | −2.27 | 4504536 | NM_000865.1 | 0.45879 | 0.00370 | 0.85 | 0.48 |
| UAP1 | NM_003115.1_PROBE1 | −0.14 | 34147515 | NM_003115.3 | 0.45424 | 0.00270 | 0.85 | 0.48 |
| RPF1 | 232020.6_PROBE1 | −1.05 | 38569467 | NM_025065.5 | 0.98900 | 0.00446 | 1.00 | 0.48 |
| C6orf162 | NM_020425.1_PROBE1 | −1.24 | 32171179 | NM_020425.3 | 0.29707 | 0.00096 | 0.81 | 0.47 |
| TIEG | NM_005655.1_PROBE1 | −1.86 | 5032176 | NM_005655.1 | 0.35958 | 0.01082 | 0.79 | 0.47 |
| TRIP12 | NM_004238.1_PROBE1 | 0.16 | 10863902 | NM_004238.1 | 0.82943 | 0.00105 | 0.96 | 0.47 |
| NIT2 | NM_020202.1_PROBE1 | −2.04 | 31543290 | NM_020202.2 | 0.90569 | 0.01585 | 0.97 | 0.46 |
| ZNF577 | NM_032679.1_PROBE1 | −2.29 | 14249251 | NM_032679.1 | 0.98697 | 0.00559 | 1.00 | 0.46 |
| KIAA0073 | AK025679_PROBE1 | −1.19 | 24308048 | NM_015342.1 | 0.23542 | 0.00065 | 0.79 | 0.46 |
| XAB1 | NM_007266.1_PROBE1 | −1.40 | 14149628 | NM_007266.1 | 0.19754 | 0.00461 | 0.73 | 0.46 |
| CCT8 | NM_006585.1_PROBE1 | 0.85 | 48762931 | NM_006585.2 | 0.52970 | 0.00281 | 0.86 | 0.46 |
| TDRD3 | NM_030794.1_PROBE1 | −2.53 | 13540575 | NM_030794.1 | 0.60651 | 0.00975 | 0.87 | 0.46 |
| LSP1 | NM_002339.1_PROBE1 | −3.00 | 10880978 | NM_002339.1 | 0.42557 | 0.00850 | 0.80 | 0.45 |
| SC5DL | NM_006918.2_PROBE1 | −1.19 | 10800413 | NM_006918.2 | 0.36378 | 0.00287 | 0.80 | 0.45 |
| NRCAM | NM_005010.1_PROBE1 | 0.22 | 41281388 | NM_005010.2 | 0.58013 | 0.00570 | 0.86 | 0.45 |
| TFB2M | 407005.3_PROBE1 | −0.84 | 11641288 | NM_022366.1 | 0.57608 | 0.00105 | 0.88 | 0.44 |
| MFHAS1 | 234824.4_PROBE1 | −0.49 | 851942 | R77310.1 | 0.45641 | 0.00209 | 0.84 | 0.44 |
| EIF1AY | NM_004681.1_PROBE1 | −0.13 | 33356162 | NM_004681.2 | 0.52152 | 0.00080 | 0.88 | 0.44 |
| NULL | AI888150_PROBE1 | −1.64 | 21754050 | AK094892.1 | 0.87570 | 0.01005 | 1.05 | 0.44 |
| ZNF235 | NM_004234.3_PROBE1 | −2.67 | 12056481 | NM_004234.3 | 0.46564 | 0.00001 | 0.90 | 0.44 |
| PSMD14 | NM_005805.1_PROBE1 | −0.35 | 42734423 | NM_005805.2 | 0.30629 | 0.00644 | 0.76 | 0.44 |
| NULL | 1045196.1_PROBE1 | −1.90 | 24719866 | CA389576.1 | 0.51972 | 0.00584 | 0.84 | 0.44 |
| MGC15397 | AW969543_PROBE1 | −2.05 | 34367087 | BX647928.1 | 0.87694 | 0.01160 | 0.95 | 0.44 |
| ZNF638 | NM_014497.1_PROBE1 | −1.44 | 21626467 | NM_014497.2 | 0.55623 | 0.00136 | 0.87 | 0.44 |
| NULL | NM_001207.1_PROBE1 | −2.64 | 4502464 | NM_001207.1 | 0.08152 | 0.00015 | 0.72 | 0.44 |
| PEX13 | NM_002618.1_PROBE1 | −2.14 | 46047483 | NM_002618.2 | 0.09870 | 0.00034 | 0.71 | 0.43 |
| NULL | 1453120.6_PROBE1 | −2.48 | | INCYTE | 0.17831 | 0.00010 | 0.78 | 0.43 |
| | | | | UNIQUE | | | | |
| ATP11B | AB023173_PROBE1 | −1.71 | 15748736 | BI757158.1 | 0.97840 | 0.00931 | 1.01 | 0.42 |
| NPM3 | NM_006993.1_PROBE1 | −1.85 | 6857817 | NM_006993.1 | 0.31244 | 0.00223 | 0.78 | 0.42 |
| PPIB | NM_000942.1_PROBE1 | −1.25 | 44890060 | NM_000942.4 | 0.51636 | 0.00423 | 0.84 | 0.42 |
| FAM51A1 | NM_017856.1_PROBE1 | −2.08 | 8923480 | NM_017856.1 | 0.72217 | 0.00179 | 1.09 | 0.42 |
| KIAA0117 | AL133010_PROBE1 | −2.86 | 38016126 | NM_015014.1 | 0.73801 | 0.02361 | 0.89 | 0.42 |
| PDLIM2 | 71831409CB1_PROBE1 | −2.89 | 40288188 | NM_021630.4 | 0.26034 | 0.01931 | 0.68 | 0.42 |
| FLJ20097 | AI904973_PROBE1 | −2.05 | 6495360 | AI904973.1 | 0.83782 | 0.01310 | 0.94 | 0.41 |
| AMACR | NM_014324.1_PROBE1 | −2.10 | 42822892 | NM_203382.1 | 0.86249 | 0.04417 | 0.93 | 0.41 |
| C14orf127 | NM_025152.1_PROBE1 | −2.74 | 13376746 | NM_025152.1 | 0.90638 | 0.00754 | 1.04 | 0.41 |
| CWF19L2 | 2902971CB1_PROBE1 | −1.65 | 22748918 | NM_152434.1 | 0.09173 | 0.00025 | 0.70 | 0.41 |
| GRK5 | NM_005308.1_PROBE1 | −1.28 | 51896033 | NM_005308.2 | 0.40743 | 0.00030 | 0.84 | 0.41 |
| SAE1 | NM_005500.1_PROBE1 | −0.73 | 4885584 | NM_005500.1 | 0.18194 | 0.00710 | 0.66 | 0.40 |
| NULL | 1377943.1_PROBE1 | −2.18 | | INCYTE | 0.09311 | 0.00051 | 0.67 | 0.40 |
| | | | | UNIQUE | | | | |
| LOC113386 | 353113.11_PROBE1 | −2.34 | 49574540 | NM_138781.2 | 0.56709 | 0.00301 | 0.85 | 0.39 |
| SCAMP3 | 3841666CB1_PROBE1 | −0.85 | 16445420 | NM_052837.1 | 0.18339 | 0.00019 | 0.75 | 0.39 |
| UBE2V2 | NM_003350.2_PROBE1 | −1.26 | 12025664 | NM_003350.2 | 0.22611 | 0.00048 | 0.75 | 0.39 |
| ENSA | 1510032.1_PROBE1 | −1.83 | 46389561 | NM_207168.1 | 0.32151 | 0.00132 | 0.77 | 0.39 |
| ZNFN1A5 | NM_022466.1_PROBE1 | −2.96 | 21314708 | NM_022466.2 | 0.36797 | 0.00098 | 0.79 | 0.39 |
| HN1 | BI759599_PROBE1 | −2.52 | 50345274 | NM_016185.2 | 0.16160 | 0.00064 | 0.70 | 0.38 |
| HSPC128 | NM_014167.1_PROBE1 | −1.75 | 7661789 | NM_014167.1 | 0.75251 | 0.00089 | 0.92 | 0.38 |
| SEC15L1 | AL137438_PROBE1 | −2.12 | 30410709 | NM_019053.2 | 0.24122 | 0.00385 | 0.70 | 0.38 |
| NULL | 1327865.1_PROBE1 | −2.93 | | INCYTE | 0.11030 | 0.00460 | 0.60 | 0.38 |
| | | | | UNIQUE | | | | |
| ZFP276 | 7399016CB_PROBE1 | −1.68 | 40805101 | NM_152287.2 | 0.35430 | 0.00027 | 0.81 | 0.38 |
| MGC4549 | NM_032377.1_PROBE1 | −1.57 | 39725656 | NM_032377.2 | 0.24504 | 0.00226 | 0.71 | 0.38 |
| PCMT1 | NM_005389.1_PROBE1 | 0.76 | 4885538 | NM_005389.1 | 0.19086 | 0.00244 | 0.68 | 0.37 |
| KIAA1432 | 244348.2_PROBE1 | −2.08 | 20521915 | AB037853.2 | 0.54488 | 0.00142 | 0.84 | 0.36 |
| NIPA2 | BC011775_PROBE1 | −0.90 | 52694674 | NM_030922.4 | 0.38579 | 0.00062 | 0.80 | 0.36 |
| MRCL3 | NM_006471.1_PROBE1 | −2.42 | 31543210 | NM_006471.2 | 0.73251 | 0.00163 | 0.91 | 0.36 |
| HPN | 785541CB1_PROBE1 | −2.75 | 4504480 | NM_002151.1 | 0.47566 | 0.00083 | 0.82 | 0.36 |
| KIAA1128 | AF241785_PROBE1 | −1.90 | 24308130 | NM_018999.1 | 0.17227 | 0.00714 | 0.62 | 0.36 |
| AD-003 | NM_014064.1_PROBE1 | −1.55 | 56676398 | NM_014064.2 | 0.15648 | 0.00506 | 0.62 | 0.36 |
| KCNK1 | NM_002245.2_PROBE1 | 3.11 | 15451900 | NM_002245.2 | 0.07382 | 0.00398 | 0.55 | 0.35 |
| NULL | 1400324.1_PROBE1 | −2.36 | 1192962 | N51796.1 | 0.39248 | 0.00069 | 0.78 | 0.33 |
| GRM8 | NM_000845.1_PROBE1 | −2.47 | 4504148 | NM_000845.1 | 0.13665 | 0.00026 | 0.67 | 0.32 |
| CST5 | NM_001900.1_PROBE1 | −2.89 | 54607081 | NM_001900.3 | 0.93726 | 0.00002 | 0.98 | 0.31 |
| SNAPC5 | 1448966.3_PROBE1 | −2.26 | 14675903 | BI222459.1 | 0.17411 | 0.00137 | 0.64 | 0.31 |
| FAM44A | 1450054.5_PROBE1 | −2.77 | 22507398 | NM_148894.1 | 0.37169 | 0.00088 | 0.76 | 0.31 |
| DKFZp434A128 | AL122120_PROBE1 | −2.72 | 6102946 | AL122120.1 | 0.84684 | 0.00759 | 1.08 | 0.31 |
| CKS1B | 1501987.1_PROBE1 | −2.06 | 4502856 | NM_001826.1 | 0.35761 | 0.00039 | 0.77 | 0.30 |
| UBE2G1 | NM_003342.1_PROBE1 | −0.82 | 33359698 | NM_003342.3 | 0.56968 | 0.00009 | 0.86 | 0.30 |
| P4HB | NM_000918.1_PROBE1 | −1.98 | 20070124 | NM_000918.2 | 0.51482 | 0.00006 | 0.84 | 0.29 |
| IK | NM_006083.2_PROBE1 | −2.84 | 11038650 | NM_006083.2 | 0.46304 | 0.00357 | 0.77 | 0.29 |
| KRT5 | NM_000424.1_PROBE1 | −1.17 | 17318577 | NM_000424.2 | 0.43037 | 0.00282 | 0.74 | 0.29 |
| LOC57821 | NM_021179.1_PROBE1 | −2.45 | 10880974 | NM_021179.1 | 0.90051 | 0.00162 | 0.95 | 0.27 |
| EXOSC3 | NM_016042.1_PROBE1 | −2.89 | 50511942 | NM_016042.2 | 0.33216 | 0.00007 | 0.77 | 0.27 |
| AIF1 | NM_032955.1_PROBE1 | −2.03 | 6680470 | NM_004847.2 | 0.49548 | 0.00726 | 0.74 | 0.27 |
| LOC90624 | AK000803_PROBE1 | −2.17 | 32171235 | NM_181705.1 | 0.71692 | 0.00107 | 0.88 | 0.26 |
| SNX6 | NM_021249.1_PROBE1 | −2.16 | 23111050 | NM_152233.1 | 0.08751 | 0.00010 | 0.57 | 0.24 |
| ORC5L | NM_002553.1_PROBE1 | −2.81 | 32454752 | NM_002553.2 | 0.62609 | 0.00092 | 0.81 | 0.23 |
| NDST2 | NM_003635.1_PROBE1 | −2.75 | 31377809 | NM_003635.2 | 0.48309 | 0.00070 | 0.76 | 0.22 |
| LRP12 | 399305.1_PROBE1 | −2.37 | 21264628 | NM_013437.2 | 0.53818 | 0.00241 | 0.76 | 0.22 |
| LIPF | NM_004190.1_PROBE1 | −5.33 | 4758675 | NM_004190.1 | 0.13309 | 0.01213 | 2.15 | 4.49 |
| NULL | 1400611.6_PROBE1 | −5.44 | | INCYTE | 0.15176 | 0.03992 | 2.11 | 3.55 |
| | | | | UNIQUE | | | | |
| C1orf42 | NM_019060.1_PROBE1 | −5.17 | 9506922 | NM_019060.1 | 0.43409 | 0.00558 | 1.32 | 3.36 |
| TMPRSS2 | NM_005656.2_PROBE1 | −5.36 | 2507612 | U75329.1 | 0.52865 | 0.01008 | 1.26 | 3.24 |
| CCR9 | NM_031200.1_PROBE1 | −5.12 | 14043043 | NM_006641.2 | 0.15220 | 0.04160 | 1.94 | 2.73 |
| GDF5 | 2222892CB1_PROBE1 | −4.86 | 5123452 | NM_000557.2 | 0.56428 | 0.02756 | 1.20 | 2.30 |
| PPP1R15B | NM_032833.1_PROBE1 | −5.10 | 14042484 | AK027650.1 | 0.23858 | 0.03629 | 1.46 | 2.25 |
| FTS | 1446648.1_PROBE1 | −4.22 | 45715990 | AL540368.3 | 0.05299 | 0.01799 | 1.82 | 2.17 |
| PSMC3 | BI225535_PROBE1 | −4.06 | 24430153 | NM_002804.3 | 0.11973 | 0.04057 | 0.72 | 1.63 |
| NULL | 1511241.2_PROBE1 | −3.51 | | INCYTE | 0.22638 | 0.02330 | 0.76 | 0.57 |
| | | | | UNIQUE | | | | |
| C6orf10 | NM_006781.1_PROBE1 | −3.48 | 31745171 | NM_006781.2 | 0.98591 | 0.01825 | 1.00 | 0.54 |
| NULL | 1498552.1_PROBE1 | −3.31 | 1815277 | AA215505.1 | 0.59473 | 0.00645 | 0.89 | 0.53 |
| ENDOGL1 | 240012.12_PROBE1 | −3.41 | 10435079 | AK023235.1 | 0.09836 | 0.04171 | 0.61 | 0.52 |
| FLJ10786 | NM_018219.1_PROBE1 | −3.46 | 8922668 | NM_018219.1 | 0.50651 | 0.01182 | 0.85 | 0.52 |
| KCNK17 | NM_031460.1_PROBE1 | −3.61 | 17025229 | NM_031460.2 | 0.36509 | 0.03969 | 0.76 | 0.51 |
| ZBED4 | 1446703.3_PROBE1 | −4.29 | 10434549 | AK022892.1 | 0.10295 | 0.02716 | 0.62 | 0.51 |
| MYO18B | NM_032608.1_PROBE1 | −3.21 | 51317365 | NM_032608.5 | 0.74848 | 0.01645 | 0.92 | 0.51 |
| NULL | 101042.1_PROBE1 | −4.19 | 10438377 | AK025762.1 | 0.52580 | 0.03085 | 0.82 | 0.51 |
| HSPCO63 | NM_014155.1_PROBE1 | −3.43 | 7661765 | NM_014155.1 | 0.51579 | 0.03531 | 0.82 | 0.50 |
| PRKWNK2 | 1027959.1_PROBE1 | −3.30 | 16199824 | BI918235.1 | 0.18278 | 0.00127 | 0.77 | 0.50 |
| TXK | 1533482CB1_PROBE1 | −4.05 | 4507742 | NM_003328.1 | 0.20389 | 0.04893 | 1.63 | 0.49 |
| TEKT2 | NM_014466.1_PROBE1 | −3.24 | 16507949 | NM_014466.2 | 0.53782 | 0.02768 | 0.83 | 0.49 |
| HSPA5 | 197393.1_PROBE1 | −3.16 | 21361242 | NM_005347.2 | 0.88098 | 0.01971 | 0.96 | 0.48 |
| NULL | 1497917.1_PROBE1 | −3.79 | 965002 | U20734.1 | 0.71763 | 0.04199 | 1.13 | 0.48 |
| GPRC5C | AK000249_PROBE1 | −3.76 | 7020202 | AK000249.1 | 0.36410 | 0.02199 | 0.76 | 0.47 |
| USP29 | NM_020903.1_PROBE1 | −3.33 | 56790915 | NM_020903.2 | 0.62441 | 0.04225 | 0.84 | 0.44 |
| SIAT1 | NM_003032.1_PROBE1 | −3.67 | 29433 | X62822.1 | 0.13479 | 0.00178 | 0.70 | 0.44 |
| MKL1 | NM_020831.1_PROBE1 | −3.42 | 47678574 | CR456522.1 | 0.22332 | 0.01142 | 0.69 | 0.43 |
| HEY2 | NM_012259.1_PROBE1 | −3.22 | 6912413 | NM_012259.1 | 0.88241 | 0.00456 | 0.96 | 0.42 |
| TTC3 | 100315.1_PROBE1 | −3.41 | 34532607 | AK126194.1 | 0.62083 | 0.00070 | 0.89 | 0.41 |
| FOXK2 | 1005690.1_PROBE1 | −3.91 | 8408272 | BE063622.1 | 0.26770 | 0.00216 | 0.73 | 0.38 |
| NPHP4 | AB014573_PROBE1 | −3.17 | 34304361 | NM_015102.2 | 0.34311 | 0.00044 | 0.80 | 0.38 |
| RPP38 | NM_006414.1_PROBE1 | −3.43 | 33859836 | NM_183005.1 | 0.85004 | 0.00793 | 1.06 | 0.38 |
| USP15 | NM_006313.1_PROBE2 | −3.43 | 14149626 | NM_006313.1 | 0.74366 | 0.00429 | 0.91 | 0.37 |
| UTRN | NM_007124.1_PROBE1 | −4.16 | 6005937 | NM_007124.1 | 0.30809 | 0.00413 | 0.74 | 0.37 |
| FLJ20245 | NM_017723.1_PROBE1 | −3.06 | 8923220 | NM_017723.1 | 0.60336 | 0.00024 | 1.13 | 0.36 |
| DKFZP434J0113 | NM_032130.1_PROBE1 | −3.71 | 14149788 | NM_032130.1 | 0.18307 | 0.01249 | 0.60 | 0.36 |
| NULL | AF112216_PROBE1 | −3.88 | | INCYTE | 0.05079 | 0.00587 | 0.51 | 0.36 |
| | | | | UNIQUE | | | | |
| NULL | K03021_PROBE1 | −4.06 | | INCYTE | 0.21872 | 0.00214 | 0.69 | 0.35 |
| | | | | UNIQUE | | | | |
| CAPN9 | NM_006615.1_PROBE1 | −3.45 | 54112395 | NM_006615.2 | 0.52096 | 0.00162 | 0.83 | 0.35 |
| PIK4CB | BM015491_PROBE1 | −3.84 | 19138365 | BM790133.1 | 0.64684 | 0.00087 | 0.88 | 0.33 |
| STAG1 | NM_005862.1_PROBE1 | −3.89 | 5032062 | NM_005862.1 | 0.12176 | 0.00046 | 0.64 | 0.31 |
| Gup1 | 7939381CB1_PROBE1 | −4.39 | 50582990 | NM_152451.2 | 0.32522 | 0.00439 | 1.41 | 0.30 |
| CORO2A | NM_003389.1_PROBE1 | −3.80 | 34335234 | NM_052820.2 | 0.64220 | 0.00142 | 0.86 | 0.28 |
| FLJ22662 | NM_024829.1_PROBE1 | −4.33 | 55743115 | NM_024829.4 | 0.34814 | 0.00045 | 1.32 | 0.26 |
| MAGI1 | BF980403_PROBE1 | −4.33 | 5811416 | AI984197.1 | 0.69796 | 0.00387 | 0.85 | 0.25 |
| NJMU-R1 | NM_022344.1_PROBE1 | −3.66 | 45505146 | NM_022344.2 | 0.23742 | 0.00773 | 0.57 | 0.25 |
| CECR5 | NM_033070.1_PROBE1 | −3.04 | 51093854 | NM_017829.5 | 0.20359 | 0.00084 | 0.58 | 0.19 |

In certain embodiments genes that are particularly useful as diagnostic/prognostic markers include genes whose expression is concordant in brain and lymphocytes. Thus, in certain embodiments bipolar disorder specific genes that are concordant in brain and lymphocytes (see, e.g., ATP6V1D, GSR, SH3GLB1, and the like), and/or schizophrenia specific genes that are concordant in brain and lymphocytes (see, e.g., PPP1R3C, CYP4F11, SCEL, and the like) are particularly useful markers.

As described herein, certain particularly relevant genes include, but are not limited to brain relevant genes, cellular growth relevant genes, apoptosis related genes, and neurogenesis related genes). The four groups overlap. For clarity, however, a “master list” taking out the overlaps is shown in Table 3.

TABLE 3
Summary of certain particularly relevant genes by functional group.

| Gene | Category |
|---|---|
| AGXT2L1 | brain relevant |
| EMX2 | brain relevant |
| SOX9 | brain relevant |
| TU3A | brain relevant |
| TUBB2B | brain relevant |
| IMPA2 | brain relevant |
| SLC1A2 | brain relevant |
| GMPR | brain relevant |
| AHNAK | brain relevant |
| ATP6V1H | brain relevant |
| MAFG | cellular growth |
| RERG | cellular growth |
| SMCY | cellular growth |
| BUB1B | Apoptosis |
| FTH1 | Apoptosis |
| IL2RA | Apoptosis |
| LGALS3 | Apoptosis |
| MT1X | Apoptosis |
| NFATC1 | Apoptosis |
| OGDH | Apoptosis |
| PPARA | Apoptosis |
| PVR | Apoptosis |
| SSPN | Apoptosis |
| TXNIP | Apoptosis |
| UNG | Apoptosis |
| EMX2 | Neurogenesis |
| ERBB2 | Neurogenesis |
| FGF2 | Neurogenesis |
| JARID2 | Neurogenesis |
| RAB23 | Neurogenesis |
| SMO | Neurogenesis |
| SOX9 | Neurogenesis |
| THBS4 | Neurogenesis |

While, in certain embodiments, the expression level of a single gene identified in Tables 1, 2, 6, 9, and/or 10 can be used as an indicator for the presence of a psychiatric disorder, as a prognostic for increased proclivity for a psychiatric disorder, and the expression level of a single gene identified in Tables 1 and 10, and/or 2 and 9, can be used as a diagnostic or prognostic indicator for schizophrenia or bipolar disorder or to distinguish between these conditions, various embodiments contemplate the use of the expression level of two or more genes identified in Tables 1, 2, 6, 9, and/or 10 for these purposes. In certain embodiments the expression levels of at least 2, 3, 4, or 5 different genes, preferably the expression levels of at least 8, 10, 15, 20, 25, 30, or 40 different genes, more preferably the expression level of at least 50, 60, or 80 different genes is determined. In certain embodiments the expression levels of at least 100, 150, or 200 different genes is determined.

II. Assays for Expression of Genes that are Indicators for a Psychiatric Disorder.

This invention identifies a number of genes, altered expression (e.g., upregulation or downregulation) of which provides an indicator of a psychiatric disorder or the predisposition thereto and/or facilitates differential diagnosis between bipolar disorder and schizophrenia.

Expression levels of a gene can be altered by changes in the copy number of the gene and/or transcription of the gene product (i.e., transcription of mRNA), and/or by changes in translation of the gene product (i.e., translation of the protein), and/or by post-translational modification(s) (e.g. protein folding, glycosylation, etc.). Thus, in various embodiments, assays of this invention typically involve assaying for level of transcribed mRNA (or other nucleic acids expressed by the genes identified herein), or level of translated protein, etc. Examples of such approaches are described below.

A) Nucleic-Acid Based Assays.

1. Target Molecules.

Changes in expression level can be detected by measuring changes in mRNA and/or a nucleic acid derived from the mRNA (e.g. reverse-transcribed cDNA, etc.). In order to measure gene expression level it is desirable to provide a nucleic acid sample for such analysis. In preferred embodiments the nucleic acid is found in or derived from a biological sample. The term “biological sample”, as used herein, refers to a sample obtained from an organism or from components (e.g., cells) of an organism. The sample may be of any biological tissue or fluid. Biological samples may also include organs or sections of tissues such as frozen sections taken for histological purposes.

It was a surprising discovery that nucleic acids derived from tissues other than neurological tissues (e.g., from blood cells) can provide effective diagnostic and/or prognostic indicators of a psychiatric disorder or a predilection to such a disorder. Thus, in certain embodiments, the biological sample is a sample comprising cells of neurological origin and/or non-neurological origin. In certain embodiments, the biological sample comprises blood cells (e.g., peripheral blood lymphocytes and/or lymphoblastic cell lines).

The nucleic acid (e.g., mRNA, or nucleic acid derived from mRNA) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In certain embodiments, the “total” nucleic acid is isolated from a given sample using, for example, an acid guanidinium-phenol-chloroform extraction method and polyA+ mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to assaying for expression level. Methods of amplifying nucleic acids are well known to those of skill in the art and include, but are not limited to polymerase chain reaction (PCR, see. e.g, Innis, et al., (1990) PCR Protocols. A guide to Methods and Application. Academic Press, Inc. San Diego,), ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA_{—}86: 1173), self-sustained sequence replication (Guatelli et al. (1990) Proc. Nat. Acad. Sci. USA 87: 1874), dot PCR, and linker adapter PCR, etc.).

In certain embodiments, where it is desired to quantify the transcription level (and thereby expression) of factor(s) of interest in a sample, the nucleic acid sample is one in which the concentration of the nucleic acids in the sample, is proportional to the transcription level (and therefore expression level) of the gene(s) of interest. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required, appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript, or large differences or changes in nucleic acid concentration are desired, no elaborate control or calibration is required.

In the simplest embodiment, the nucleic acid sample is the total mRNA or a total cDNA isolated and/or otherwise derived from a biological sample (e.g., a sample from a neural cell or tissue). The nucleic acid may be isolated from the sample according to any of a number of methods well known to those of skill in the art as indicated above.

2. Hybridization-Based Assays.

Using the known sequence(s) of the various genes identified in Tables 1, 2, 6, 9, and 10 detecting and/or quantifying the transcript(s) can be routinely accomplished using nucleic acid hybridization techniques (see, e.g., Sambrook et al. supra). For example, one method for evaluating the presence, absence, or quantity of reverse-transcribed cDNA involves a “Southern Blot”. In a Southern Blot, the DNA (e.g., reverse-transcribed mRNA), typically fragmented and separated on an electrophoretic gel, is hybridized to a probe specific for the target nucleic acid. Comparison of the intensity of the hybridization signal from the target specific probe with a “control” probe (e.g. a probe for a “housekeeping gene) provides an estimate of the relative expression level of the target nucleic acid.

Alternatively, the mRNA transcription level can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes can be used to identify and/or quantify the target mRNA. Appropriate controls (e.g. probes to housekeeping genes) can provide a reference for evaluating relative expression level.

An alternative means for determining the gene expression level(s) is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use can vary depending on the particular application.

In some applications it is necessary to block the hybridization capacity of repetitive sequences. Thus, in some embodiments, tRNA, human genomic DNA, or Cot-1 DNA is used to block non- specific hybridization.

3. Amplification-Based Assays.

In another embodiment, amplification-based assays can be used to measure transcription level(s) of the various genes identified herein. In such amplification-based assays, the target nucleic acid sequences act as template(s) in amplification reaction(s) (e.g. Polymerase Chain Reaction (PCR) or reverse-transcription PCR (RT-PCR)). In a quantitative amplification, the amount of amplification product will be proportional to the amount of template in the original sample. Comparison to appropriate (e.g. healthy tissue or cells unexposed to the test agent) controls provides a measure of the transcript level.

Methods of “quantitative” amplification are well known to those of skill in the art are Illustrated in Example 1. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). One approach, for example, involves simultaneously co-amplifying a known quantity of a control sequence using the same primers as those used to amplify the target. This provides an internal standard that may be used to calibrate the PCR reaction.

One suitable internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al. (1990) Academic Press, Inc. N.Y. The known nucleic acid sequence(s) for the genes identified herein are sufficient to enable one of skill to routinely select primers to amplify any portion of the gene.

4. Hybridization Formats and Optimization of Hybridization

### a. Array-Based Hybridization Formats.

In certain embodiments, the methods of this invention can be utilized in array-based hybridization formats. Arrays typically comprise a multiplicity of different “probe” or “target” nucleic acids (or other compounds) attached to one or more surfaces (e.g., solid, membrane, or gel). In certain embodiments, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays, can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

The simple spotting, approach has been automated to produce high density spotted arrays (see, e.g., U.S. Pat. No: 5,807,522). This patent describes the use of an automated system that taps a microcapillary against a surface to deposit a small volume of a biological sample. The process is repeated to generate high density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high density arrays is also described in U.S. Pat. Nos. 5,744,305, 5,800,992 and 5,445,934. In addition, a number of high density arrays are commercially available.

### b. Other Hybridization Formats.

As indicated above a variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labelled probes or the like. Other labels include ligands that bind to labeled antibodies, fluorophores, chemi-luminescent agents, enzymes, and antibodies which can serve as specific binding pair members for a labeled ligand.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario), Q Beta Replicase systems, or branched DNA amplifier technology commercialized by Panomics, Inc. (Fremont Calif.), and the like.

### e. Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or

RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results, and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

### f. Labeling and Detection of Nucleic Acids.

The probes used herein for detection of gene expression levels can be full length or less than the full length of the mRNA(s). Shorter probes are empirically tested for specificity. Preferred probes are sufficiently long so as to specifically hybridize with the target nucleic acid(s) under stringent conditions. The preferred size range is from about 20 bases to the full length of the encoding mRNA, more preferably from about 30 bases to the length of the mRNA, and most preferably from about 40 bases to the length of mRNA.

The probes are typically labeled, with a detectable label. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another.

Suitable chromogens which can be employed include those molecules and compounds which absorb light in a distinctive range of wavelengths so that a color can be observed or, alternatively, which emit light when irradiated with radiation of a particular wave length or wave length range, e.g., fluorescers.

Desirably, fluorescent labels should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye can differ from the unbound dye. Therefore, when referring to the various wavelength ranges and characteristics of the dyes, it is intended to indicate the dyes as employed and not the dye which is unconjugated and characterized in an arbitrary solvent.

Detectable signal can also be provided by chemiluminescent and bioluminescent sources. Chemiluminescent sources include a compound which becomes electronically excited by a chemical reaction and can then emit light which serves as the detectable signal or donates energy to a fluorescent acceptor. Alternatively, luciferins can be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label can be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are easily added during an in vitro transcription reaction. Thus, for example, fluorescein labeled UTP and CTP can be incorporated into the RNA produced in an in vitro transcription.

The labels can be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe-CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

B) Polypeptide-Based Assays.

In various embodiments the peptide(s) encoded by one or more genes listed in Tables 1, and/or 2, and/or 6, and/or 9, and/or 10 can be detected and quantified to provide a measure of expression level. Protein expression can be measured by any of a number of methods well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In one preferred embodiment, the polypeptide(s) are detected/quantified in an electrophoretic protein separation (e.g., a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of polypeptide(s) of this invention in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target polypeptide(s) and can be directly labeled or alternatively may be subsequently detected using labeled antibodies (e.g., labeled sheep anti-mouse antibodies) that specifically bind to the a domain of the antibody.

In preferred embodiments, the polypeptide(s) are detected using an immunoassay. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Ten (1991) Basic and Clinical Immunology 7th Edition.

Immunological binding assays (or immunoassays) typically utilize a “capture agent” to specifically bind to and often immobilize the analyte(s). In preferred embodiments, the capture agent is an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent /polypeptide complex.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured analyte is directly measured. In one preferred “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and the sample is then contacted with a capture agent. The amount of labeled polypeptide bound to the antibody is inversely proportional to the concentration of target polypeptide present in the sample.

In one embodiment, the antibody is immobilized on a solid substrate. The amount of target polypeptide bound to the antibody may be determined either by measuring the amount of target polypeptide present in an polypeptide /antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind the target peptide(s) either alone or in combination. In the case where the antibody that binds the target polypeptide(s) is not labeled, a different detectable marker, for example, an enzyme-labeled antibody capable of binding to the monoclonal antibody which binds the target polypeptide, can be employed. Any of the known modifications of EIA, for example, enzyme-linked immunoabsorbent assay (ELISA), may also be employed. As indicated above, also contemplated by the present invention are immunoblotting immunoassay techniques such as western blotting employing an enzymatic detection system.

The immunoassay methods of the present invention can also include other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutination with antibody-coated or antigen-coated red blood corpuscles, and immunoassays employing an avidin-biotin or streptavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds the target polypeptide is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents can also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein, are commercially available or can be produced using standard methods well know to those of skill in the art.

It will also be recognized that antibodies can be prepared by any of a number of commercial services (e.g., Berkeley antibody laboratories, Bethyl Laboratories, Anawa, Eurogenetec, etc.).

C) Assay Optimization.

The assays of this invention have immediate utility as prognostic and/or diagnostic assays as described herein, or in screening for agents useful for the treatment of a psychiatric disorder (e.g., schizophrenia and/or bipolar disorder). The assays of this invention can be optimized for use in particular contexts, depending, for example, on the source and/or nature of the biological sample and/or the particular test agents, and/or the analytic facilities available. Thus, for example, optimization can involve determining optimal conditions for binding assays, optimum sample processing conditions (e.g. preferred PCR conditions), hybridization conditions that maximize signal to noise, protocols that improve throughput, etc. In addition, assay formats can be selected and/or optimized according to the availability of equipment and/or reagents. Thus, for example, where commercial antibodies or ELISA kits are available it may be desired to assay protein concentration. Conversely, where it is desired to screen for modulators that alter transcription nucleic acid based assays are preferred.

Routine selection and optimization of assay formats is well known to those of ordinary skill in the art.

D) Assay Scoring.

In various embodiments, the the assays of this invention level are deemed to show a positive result, when the expression level (e.g., transcription, translation) of the gene(s) is upregulated or downregulated as shown in the tables herein. In certain embodiments this is determined with respect to the level measured or known for a control sample (e.g. either a level known or measured for a normal healthy cell, tissue or organism mammal of the same species and/or sex and/or age), or a “baseline/reference” level determined at a different tissue and/or a different time for the same individual). In a particularly preferred embodiment, the assay is deemed to show a positive result when the difference between sample and “control” is statistically significant (e.g. at the 85% or greater, preferably at the 90% or greater, more preferably at the 95% or greater and most preferably at the 98% or 99% or greater confidence level).

III. Screening for Agents that Mitigate One or More Symptoms of a Psychiatric Disorder.

In certain embodiments this invention provides methods of screening for agents that mitigate one or more symptoms of a psychiatric disorder. The methods typically involve administering one or more test agent to a cell and/or to a mammal; and detecting altered expression in said cell and/or mammal of two or more genes listed in Table 1, and/or Table 2, and or Table 6, and/or Table 9, and/or Table 10, where upregulation or downregulation (as indicated in Table 1, and/or Table 2, and or Table 6, and/or Table 9, and/or Table 10) of expression of said two or more genes, as compared to a control, is an indicator that said test agent(s) have activity that mediates one or more symptoms of a psychiatric disorder.

Methods of screening for expression level of one or more gene are known to those of skill in the art and are also described above.

The screening assays are amenable to “high-throughput” modalities. Conventionally, new chemical entities with useful properties (e.g., modulation of expression of one or more of the genes identified herein) are generated by identifying a chemical compound (called a “lead compound”) with the desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A) Combinatorial Chemical Libraries

In certain embodiments, combinatorial chemical libraries can be used to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

B) High Throughput Assays of Chemical Libraries.

Any of the assays for agents that modulate expression and/or activity of one or more of the genes described herein are amenable to high throughput screening. As described above, having determined that these components/pathways are associated with the molecular mechanisms underlying addiction, it is believe that modulators can have significant therapeutic value. Certain preferred assays detect increases of transcription (i.e., increases of mRNA production) by the test compound(s), increases of protein expression by the test compound(s), or binding to the gene (e.g., gDNA, or cDNA) or gene product (e.g., mRNA or expressed protein) by the test compound(s).

High throughput assays for the presence, absence, or quantification of particular nucleic acids or protein products are well known to those of skill in the art. Similarly, binding assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

### IV. Kits.

In still another embodiment, this invention provides kits for practice of the assays or use of the compositions described herein. In one preferred embodiment, the kits probe nucleic acids (e.g., in a nucleic acid array) to hybridize to the mRNAs described herein. In certain embodiments the kits comprise antibodies that specifically bind to one or more of the proteins encoded by the genes identified herein. The kits can optionally include any reagents and/or apparatus to facilitate practice of the assays described herein. Such reagents include, but are not limited to buffers, labels, labeled antibodies, labeled nucleic acids, filter sets for visualization of fluorescent labels, blotting membranes, and the like.

In addition, the kits can optionally include instructional materials containing directions (i.e., protocols) for the practice of the assay methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### V. Modulator Databases.

In certain embodiments, the agents that score positively in the assays described herein (e.g. show an ability to alter expression and/or activity of one or more genes as described herein) can be entered into a database of putative modulators for use in a psychiatric disorder. The term database refers to a means for recording and retrieving information. In certain embodiments the database also provides means for sorting and/or searching the stored information. The database can comprise any convenient media including, but not limited to, paper systems, card systems, mechanical systems, electronic systems, optical systems, magnetic systems or combinations thereof. Typical databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

In certain embodiments this invention also contemplates databases comprising one or more (typically at least 2, 5, or 10 or more, preferably 20, 40, 60, or 80 or more, more preferably 100 or more or even all) of the gene(s) identified herein. The database preferably further includes information regarding the upregulation or downregulation of the expression of the gene(s) in a psychiatric disorder (e.g., schizophrenia, bipolar disorder, etc.).

This invention also contemplates the use of such databases in computer systems and/or chips to provide data upon placement of a query, e.g. in response to a screening assay.

### Examples

The following examples are offered to illustrate, but not to limit the claimed invention.

### Example 1

### Shared Pathway Alterations in Schizophrenia And Bipolar Disorder In DLPFC Involve Cellular Growth Related Functions

Schizophrenia and bipolar disorder together affect approximately 2.5% of the world population and the etiologies are thought to involve multiple genetic variations. Microarray technology allows for the simultaneous analysis of gene expression patterns in thousands of genes that may provide a characteristic signature for a brain disorder. The Stanley Array DLPFC Set A Collection (BA 46) was tested by microarray analysis. Subjects with schizophrenia (SZ, n=32) and bipolar disorder (BPD, n=27), and controls (n=29) were tested on the Codelink UniSet Human 20K Bioarray platform. Selected transcripts were further assayed with quantitative real time PCR. The strong effects of age, gender, and pH in the analysis of differential gene expression were controlled by ANCOVA. Two criteria were established for differential gene expression: 1) significantly dysregulated in both BPD and SZ compared to controls, and 2) significant in ANCOVA analysis with samples that have a restricted-pH and in an ANCOVA with all samples unrestricted-pH. A working list of 82 candidate genes passed these two criteria and this set of genes was over-represented for functional category of Cellular Growth and Proliferation (p=3.19×10⁻¹⁹ uncorrected for multiple testing). Two related functional subcategories were also over-represented in BPD and SZ: Nervous System Development and Function (quantity of neuroglia, quantity of neurons, neurogenesis, development of nervous system; p-values=8.34×10⁻⁰⁶) and Cell Death (p-value=2.98×10⁻⁸). Eight genes dysregulated in both BPD and SZ were confirmed with QPCR, and three of these were brain enriched genes (AGXT2L1, SLC1A2, and TU3A). The distribution of AGXT2L1 expression in controls versus psychiatric BPD and Sz was highly significant (Fisher's Exact Test, p<1×10⁻⁶), based upon the number of subjects above median expression of AGXT2L1. These results suggest a common molecular phenotype in both disorders and offer a window into discovery of common pathophysiology that might lead to core treatments. At the same time, divergent expression profiles suggest a vast region of unshared molecular phenotype and dysregulation.

### Methods

Total RNA

RNA samples (n=105) from the dorsolateral prefrontal cortex (BA 46) Micorarray Collection Set A were received from the Stanley Medical Research Institute (SMRI, Bethesda, Md.): 35 schizophrenia (SZ), 35 bipolar disorder (BPD) and 35 controls. In the final analysis, there were 88 subjects analyzed including 32 SZ, 29 BPD, and 27 controls; while 17 samples were not included in the analysis for reasons described (see Results). The demographics for all subjects and statistical summaries of the 88 subjects analyzed are shown (Table 4).

TABLE 4
Demographic variables for samples in SMRI microarray collection A
(BA 46). There were 105 RNA samples received for microarray analysis, of which
88 samples were used. The summary statistics were calculated for those
samples that were included in the final analysis. In column 3, 1 = male and
2 = female. The final column labeled “analysis” identifies if a
subject was included or excluded from the study. “Yes” means that the
sample was included in the final set of 88 samples that were analyzed. A sample
with any other designation(s) in the ‘“Analysis” column was judged to be
an “outlier” due to one or more of the following criteria: 1) low yield of
cRNA synthesis; 2) outlier on a principal component analysis; 3) high
number of genes with low signal intensity; and was not included in the
microarray analysis of 88 subjects. These subjects were dropped blindly
from the study, and were further not used in real time PCR, to keep the subject
sets consistent.

| | | | PMI | Brain | RI | cRNA | rRNA | |
|---|---|---|---|---|---|---|---|---|
| Group | Age | Gender | (hr) | pH | (hr) | (nt) | 28S/18S | Analysis |
| Schizophrenia | 45 | 2 | 52 | 6.51 | 12 | 750 | 2.89 | yes |
| Schizophrenia | 40 | 1 | 34 | 6.18 | 2 | 700 | 2.19 | yes |
| Schizophrenia | 51 | 1 | 43 | 6.63 | 4 | 750 | 1.72 | yes |
| Schizophrenia | 19 | 1 | 28 | 6.73 | 11 | 950 | 2.46 | yes |
| Schizophrenia | 53 | 2 | 13 | 6.49 | 3 | 800 | 2.07 | yes |
| Schizophrenia | 37 | 1 | 30 | 6.8 | 3 | 900 | 1.92 | yes |
| Schizophrenia | 24 | 1 | 15 | 6.2 | 5 | 850 | 3.07 | yes |
| Schizophrenia | 44 | 1 | 9 | 5.9 | 4 | 700 | 1.82 | yes |
| Schizophrenia | 39 | 1 | 80 | 6.6 | 8 | 900 | 2.4 | yes |
| Schizophrenia | 33 | 1 | 29 | 6.5 | 5 | 900 | 1.97 | yes |
| Schizophrenia | 50 | 1 | 9 | 6.2 | 1 | 600 | 1.75 | yes |
| Schizophrenia | 43 | 1 | 18 | 6.3 | 2 | 650 | 2.28 | yes |
| Schizophrenia | 32 | 2 | 36 | 6.8 | 5 | 700 | 2.14 | yes |
| Schizophrenia | 35 | 1 | 47 | 6.4 | 6 | 850 | 2.13 | yes |
| Schizophrenia | 44 | 1 | 32 | 6.67 | NA | 900 | 1.68 | yes |
| Schizophrenia | 47 | 1 | 13 | 6.3 | 1 | 650 | 1.78 | yes |
| Schizophrenia | 45 | 1 | 35 | 6.66 | 9 | 750 | 2.31 | yes |
| Schizophrenia | 36 | 2 | 27 | 6.49 | 4 | 800 | 1.77 | yes |
| Schizophrenia | 53 | 1 | 38 | 6.17 | 13 | 650 | 1.88 | yes |
| Schizophrenia | 54 | 2 | 42 | 6.65 | 13 | 750 | 2.49 | yes |
| Schizophrenia | 47 | 2 | 30 | 6.47 | 3 | 850 | 1.18 | yes |
| Schizophrenia | 39 | 1 | 26 | 6.8 | 6 | 850 | 2.2 | yes |
| Schizophrenia | 38 | 1 | 35 | 6.68 | 8 | 900 | 2.53 | yes |
| Schizophrenia | 41 | 1 | 54 | 6.18 | 5 | 500 | 2.47 | yes |
| Schizophrenia | 42 | 1 | 26 | 6.19 | 2 | 900 | 2.05 | yes |
| Schizophrenia | 47 | 2 | 35 | 6.5 | 10 | 900 | 1.78 | yes |
| Schizophrenia | 42 | 1 | 19 | 6.48 | 3 | 900 | 2.9 | yes |
| Schizophrenia | 46 | 1 | 30 | 6.72 | 9 | 800 | 1.72 | yes |
| Schizophrenia | 59 | 2 | 38 | 6.93 | 10 | 650 | 3.81 | yes |
| Schizophrenia | 52 | 1 | 16 | 6.52 | 2 | 700 | 2.32 | yes |
| Schizophrenia | 52 | 1 | 10 | 6.1 | 2 | 500 | 2.53 | yes |
| Schizophrenia | 44 | 2 | 26 | 6.58 | 2 | 950 | 1.94 | yes |
| Schizophrenia | 43 | 1 | 26 | 6.42 | 4 | NA | 1.72 | low cRNA |
| Schizophrenia | 31 | 1 | 33 | 6.2 | 7 | NA | 2.43 | low cRNA |
| Schizophrenia | 43 | 1 | 65 | 6.67 | 19 | NA | 3.46 | low cRNA |
| Bipolar Disorder | 29 | 1 | 48 | 6.39 | 3 | 800 | 3.04 | yes |
| Bipolar Disorder | 29 | 2 | 62 | 6.74 | 5 | 700 | 1.77 | yes |
| Bipolar Disorder | 45 | 1 | 28 | 6.35 | 3 | 900 | 2.09 | yes |
| Bipolar Disorder | 44 | 1 | 19 | 6.74 | 5 | 900 | 2.17 | yes |
| Bipolar Disorder | 48 | 2 | 18 | 6.5 | 4 | 850 | 1.79 | yes |
| Bipolar Disorder | 42 | 1 | 32 | 6.65 | 3 | 950 | 0.49 | yes |
| Bipolar Disorder | 59 | 2 | 53 | 6.2 | 27 | 800 | 2.04 | yes |
| Bipolar Disorder | 54 | 1 | 44 | 6.5 | 29 | 800 | 2.31 | yes |
| Bipolar Disorder | 58 | 2 | 35 | 6.5 | 7 | 800 | 3.1 | yes |
| Bipolar Disorder | 41 | 1 | 39 | 6.6 | 19 | 750 | 2.57 | yes |
| Bipolar Disorder | 64 | 1 | 16 | 6.1 | 1 | 600 | 4.18 | yes |
| Bipolar Disorder | 59 | 1 | 84 | 6.65 | 12 | 700 | 3.3 | yes |
| Bipolar Disorder | 51 | 1 | 23 | 6.67 | 4 | 850 | 1.94 | yes |
| Bipolar Disorder | 56 | 2 | 26 | 6.58 | 10 | 750 | 3.64 | yes |
| Bipolar Disorder | 35 | 1 | 22 | 6.58 | 4 | 1000 | 2.17 | yes |
| Bipolar Disorder | 50 | 2 | 62 | 6.51 | 14 | 700 | 2.51 | yes |
| Bipolar Disorder | 49 | 2 | 38 | 6.39 | 2 | 900 | 2.06 | yes |
| Bipolar Disorder | 33 | 2 | 24 | 6.51 | 4 | 900 | 3.54 | yes |
| Bipolar Disorder | 41 | 2 | 28 | 6.44 | 14 | 700 | 3.25 | yes |
| Bipolar Disorder | 43 | 2 | 57 | 5.92 | 15 | 550 | 2.65 | yes |
| Bipolar Disorder | 56 | 1 | 23 | 6.07 | 3 | 500 | 1.71 | yes |
| Bipolar Disorder | 29 | 1 | 60 | 6.7 | 10 | 1000 | 1.7 | yes |
| Bipolar Disorder | 42 | 2 | 49 | 6.65 | 15 | 600 | 1.69 | yes |
| Bipolar Disorder | 48 | 1 | 23 | 6.9 | 6 | 750 | 2.49 | yes |
| Bipolar Disorder | 41 | 1 | 70 | 6.71 | 4 | 900 | 1.91 | yes |
| Bipolar Disorder | 35 | 1 | 35 | 6.3 | 6 | 1000 | 1.93 | yes |
| Bipolar Disorder | 43 | 2 | 39 | 6.74 | 24 | 800 | 2.27 | yes |
| Bipolar Disorder | 19 | 1 | 12 | 5.97 | 8 | 600 | 1.71 | outlier |
| Bipolar Disorder | 35 | 2 | 17 | 6.1 | 3 | 700 | 1.73 | outlier |
| Bipolar Disorder | 51 | 2 | 77 | 6.42 | 54 | 550 | 1.54 | outlier |
| Bipolar Disorder | 44 | 2 | 37 | 6.37 | 10 | 500 | 1.01 | outlier |
| Bipolar Disorder | 45 | 1 | 35 | 6.03 | 6 | 600 | 2.49 | outlier |
| Bipolar Disorder | 49 | 2 | 19 | 5.87 | 10 | NA | 2.55 | low cRNA |
| Bipolar Disorder | 55 | 2 | 41 | 5.76 | 4 | NA | 2.3 | low cRNA |
| Bipolar Disorder | 63 | 2 | 32 | 6.97 | 6 | NA | 1.91 | low cRNA |
| Control | 49 | 1 | 46 | 6.5 | 3 | 800 | 2.07 | yes |
| Control | 53 | 1 | 9 | 6.4 | 2 | 600 | 2.72 | yes |
| Control | 51 | 1 | 31 | 6.7 | 2 | 1200 | 1.74 | yes |
| Control | 53 | 1 | 28 | 6 | 2 | 300 | 2.43 | yes |
| Control | 35 | 1 | 52 | 6.7 | 3 | 800 | 3.74 | yes |
| Control | 34 | 1 | 22 | 6.48 | 1 | 1200 | 1.73 | yes |
| Control | 47 | 1 | 21 | 6.81 | 2 | 1000 | 2.17 | yes |
| Control | 45 | 1 | 29 | 6.94 | 4 | 850 | 2.28 | yes |
| Control | 34 | 2 | 24 | 6.87 | 2 | 750 | 2.36 | yes |
| Control | 42 | 1 | 37 | 6.91 | NA | 800 | 1.63 | yes |
| Control | 44 | 2 | 10 | 6.2 | NA | 700 | 1.71 | yes |
| Control | 45 | 1 | 18 | 6.81 | 2 | 900 | 2.8 | yes |
| Control | 49 | 1 | 23 | 6.93 | 4 | 900 | 2.1 | yes |
| Control | 35 | 1 | 24 | 7.03 | 2 | 750 | 1.79 | yes |
| Control | 55 | 1 | 31 | 6.7 | 4 | 700 | 2.69 | yes |
| Control | 49 | 2 | 45 | 6.72 | 3 | 750 | 2.15 | yes |
| Control | 48 | 1 | 31 | 6.86 | 3 | 500 | 2.41 | yes |
| Control | 50 | 1 | 49 | 6.75 | 6 | 900 | 2.05 | yes |
| Control | 32 | 1 | 13 | 6.57 | 6 | 800 | 2.22 | yes |
| Control | 47 | 1 | 11 | 6.6 | 3 | 700 | 2.12 | yes |
| Control | 46 | 1 | 31 | 6.67 | | 700 | 1.8 | yes |
| Control | 40 | 1 | 38 | 6.67 | 9 | 1000 | 2.39 | yes |
| Control | 51 | 1 | 22 | 6.71 | 7 | 900 | 2.75 | yes |
| Control | 48 | 1 | 24 | 6.91 | 6 | 800 | 2.58 | yes |
| Control | 44 | 2 | 28 | 6.59 | 3 | 900 | 2.22 | yes |
| Control | 39 | 2 | 58 | 6.46 | 14 | 900 | 2.76 | yes |
| Control | 47 | 1 | 36 | 6.57 | 2 | 850 | 1.97 | yes |
| Control | 37 | 1 | 13 | 6.5 | 2 | 650 | 2.19 | yes |
| Control | 38 | 2 | 33 | 6 | 3 | 750 | 2.04 | yes |
| Control | 38 | 2 | 28 | 6.7 | 3 | 700 | 2.43 | outlier |
| Control | 60 | 1 | 47 | 6.8 | 4 | 500 | 1.81 | outlier |
| Control | 33 | 2 | 29 | 6.52 | 3 | 500 | 1.63 | outlier |
| Control | 31 | 1 | 11 | 6.13 | 3 | 700 | 2.19 | outlier |
| Control | 41 | 2 | 50 | 6.17 | 2 | 700 | 1.99 | outlier |
| Control | 57 | 1 | 26 | 6.4 | 0 | NA | 1.2 | low cRNA |

| Mean (SD) | | | | | | | |
|---|---|---|---|---|---|---|---|
| Analysed | | Gender | | Brain | | | |
| Subjects | Age | M/F | PMI | pH | RI | cRNA | rRNA |
| Schizophrenia | 42.9 | 23/9 | 30.5 | 6.48 | 5.5 | 778 | 2.19 |
| | (8.6) | | (15.1) | (0.25) | (3.6) | (124) | (0.50) |
| Bipolar Disorder | 45.3 | 15/12 | 39.1 | 6.5 | 9.3 | 794 | 2.38 |
| | (9.8) | | (17.9) | (0.23) | (7.8) | (138) | (0.77) |
| Control | 44.4 | 23/6 | 28.9 | 6.64 | 3.8 | 805 | 2.26 |
| | (6.5) | | (12.7) | (0.26) | (2.8) | (181) | (0.44) |

p-value

| | | | | | | | |
|---|---|---|---|---|---|---|---|
| Schizophrenia- | 0.452 | 0.31 | 0.653 | 0.016 | 0.049 | 0.504 | 0.565 |
| Control | | | | | | | |
| Bipolar-Control | 0.674 | 0.003 | 0.018 | 0.041 | 0.002 | 0.801 | 0.487 |

Codelink 20K Oligonucleotide Microarrays

The gene expression profile for each subject was individually measured with a Codelink UniSet Human 20K I Bioarray (GE Amersham Biosciences, Chandler Ariz.). This array contains 20,289 probes which are 30-mers spotted on glass, representing 19,881 discovery genes. There are 108 positive, 300 negative and 72 other probes used as chip quality control probes. The cRNA and bioarray hybridizations were performed according to Codelink protocol (GE Amersham Biosciences). In brief, 2 μg of total RNA from each sample was transformed into cDNA by reverse-transcription, and synthesized to biotinylated cRNA by in vitro transcription. Ten μg of cRNA was fragmented and applied to the Codelink UniSet Human 20K I Bioarray glass slide. The fluorescent hybridization signal was scanned with a GenePix/4000B scanner (Motorola) and processed with Codelink Expression v4.1 software.

Microarray Data Analysis

The microarray raw intensity for each gene after correction for background (spot mean—local background median for each spot) was exported from Codelink Expression v4.1 and transformed to log2 format. After log2 transformation, a normalization was performed by forming a ratio of each gene to the array median. The median of each array was chosen after eliminating genes with ≦0 expression across all selected arrays. All spots were labeled in the software with a quality flag as Good (G), Contaminated (C), Irregular (I), Near Background (L), or Saturated (S) according to the manufacturer's preset parameters.

Potential outlier microarrays chip were assessed by agreement among the following procedures. The control probes across all subjects were analyzed for each chip, and then the analysis was extended to discovery genes good quality spots (G only), and then to all quality spots. The outlier chips were determined by PCA plots (Partek Genomics Suite, v 6.2, St Louis, Mo.) of all subjects' good quality discovery genes, by the expression profiles of positive control probes, by an average correlation index (Tomita, Vawter et al. 2004), and by deviations from a virtual Median Chip (calculated from the median raw data for each gene across control subjects' chips) using a linear regression plot to show profiles for each chip.

There were 6 samples without sufficient cRNA to hybridize to microarrays after 2 separate syntheses (3 SZ, 2 BPD, 1 control), these are shown in Table 4 in the last column as “low cRNA”. Among the remaining 99 bioarray chips hybridized with cRNA, 11 chips were excluded as outlier chips from further data analysis with the above outlier methods (5 controls and 6 BPD cases).

In all ANCOVAs gender and diagnosis were considered as main effects, age and tissue pH were considered as covariates, to estimate the adjusted mean expression for each gene. Planned contrasts between adjusted means for BPD and Controls, and SZ and Controls with p-value<0.05 was chosen to select significant genes for further studies. A secondary ANCOVA with the same parameters with subjects restricted to a pH above the median pH of 6.57 was performed. This restricted analysis was compared to the unrestricted analysis to enrich the list of genes with diagnosis effects relative to strong pH sensitive genes (Vawter et al. (2006) Mol. Psychiatry. 11(7): 663-679). The ANCOVA p-values were adjusted with Benjamini-Hochberg false discovery method Benjamini and Hochberg (1995) J. Royal Statistical Soc., Series B-Methodological 57(1): 289-300), although in this study there were no main effect p-values that were significant following ANCOVA. As a check for correct assignment of gender and the running of chips gender genes were evaluated for XIST and RPS4Y (Vawter et al. (2004) Neuropsychopharmacology 29(2): 373-384).

After assembly of a final differential expression list of genes in both BPD and SZ that passed multiple filters a final check for the combined effects of refrigeration interval, RNA quality, PMI, age, gender, diagnosis, and pH was made within one ANCOVA. Although this multifactored ANCOVA could have been used originally, for our discovery purpose we wished to find genes that appeared to be least sensitive to age, gender, and pH effects before performing a final polished analysis with the additional demographic covariates.

Real Time Quantitative PCR

The genes that were differentially expressed in both SZ and BPD by microarray were selected for further testing by quantitative real-time PCR with SybrGreen dye. DNA was removed from each total RNA sample with a TURBO DNase-Free Kit (Ambion, #1970) following the manufacturer's protocol for rigorous DNase treatment. Briefly, 2.5 μg total RNA (˜1 μg/μl) for each sample was cleaned in 10 μl reaction volume with 1 μl 10× TURBO DNase Buffer and 2 μl TURBO DNase. After incubation at 37° C. for 30 min, the DNAse was removed by 2 μl DNAse inactivation reagent. The mixture was incubated for 2 min at RT, centrifuged at 10,000×g for 1.5 min at RT, the supernatant consisting of about 10 μl RNA was reverse transcribed into first strand cDNA with oligo-(dT)₁₆ primers in 100 μl reaction volume with Taqman Reverse Transcription Reagents (Applied Biosystems, N808-0234, Foster City Calif.) according to the manufacturer's two-step RT-PCR procedures.

Primers were designed with Primer Express (ABI) near the array probe provided by CodeLink. Each primer set was BLAST searched against the entire human genomic sequence database for specificity (with significant alignments E value<10⁻³).

Athough each RNA sample was first DNAsed, to further increase gene specificity most primers were designed to span exons to eliminate amplification of any residual genomic DNA contamination. For some primers, exon spanning decreased the BLAST specificity, therefore primers were designed to be close to array probes within a single exon. The dissociation curves of real time PCR were monitored for primer-dimer pairings, which interfere with SybrGreen fluorescence measurements. The primer sequence for each gene tested by QPCR is available upon request from the authors.

The real-time PCR was performed in an Applied Biosystems 7000 sequence detection instrument (ABI, Foster City, Calif., USA) using SybrGreen PCR Master Mix (ABI) with 25 μl reaction volume and 5 μl diluted cDNA template. The delta Ct method was used to calculate the relative fold change. CRSP9 was chosen as reference gene to normalize the Q-PCR data as the fold change was close to 1 in both SZ and BPD means compared to control's array data. The simple t-test (two-tailed with unequal variance) was used for detection of significant changes in expression for each gene. The genes for Q-PCR validation were chosen by the criteria that they meet significant differential expression after adjustment for multiple covariates (p<0.05 by ANCOVA) with fold change greater than ±1.25 for comparisons of SZ and control group, and BPD and control group.

Bioinformatics

The differentially expressed gene list was obtained by meeting two criteria: 1) intersection of both bipolar disorder and schizophrenia for significant genes, and 2) passed two ANCOVAs for restricted pH>6.57 and unrestricted ANCOVA for all pH. A tertiary criterion for genes was that pass a and b was examined for correlation with a brain enriched gene that would increase the potential relevance to the pathophysiology of both disorders. To determine the brain enrichment compared to other tissue expression levels, each gene was examined in the Novartis website “//symatlas.gnf.org/SymAtlas” (Su et al. (2004) Proc. Natl. Acad. Sci., USA, 101(16): 6062-6067).

Multiple classification models were run to predict membership of subjects into either psychiatric disorder or control groups. When discriminant analysis was run with the final gene list and all 88 samples, the result showed 100% correct classification. However, it was recommended to use a nested cross-validation model by randomly leaving out samples in a training set and running separate predictions on the left out samples (Partek, Genomics Solution v 6, St. Louis Mo.). For a 2 level nested cross-validation model, an inner 11-partition of the data followed by an outer 4-partition model was run to obtain the normalized correct rate of predictions. The average normalized correct rate of predictions for the nested double cross validation model was reported.

The distribution of the final list of 82 significant genes in different biochemical and functional pathways was analyzed with Ingenuity Pathway Analysis version 4 (Ingenuity, Redwood City, Calif.) or EASE (Hosack et al. (2003) Genome Biol., 4(10): R70). For Ingenuity, a Fisher's Exact Test was generated based upon submitting a list of 82 genes shared between BPD and SZ and looking at the total number of functional pathways mapped in the ingenuity database to the 82 genes. the proportion of genes in the functional pathway was compared to the proportion of genes in the submitted list. The p-values were not corrected for multiple pathway testing to reduce false negatives, however only the highest p-values were reported which would likely pass conventional multiple correction.

Genotyping

A validated TaqMan genotyping assay for AGXT2L1 (alanine-glyoxylate aminotransferase 2-like 1) dbSNP rs1377210 was run on the samples utilized for gene expression. The genotyping assay (ABI ID#C_{—}8748585_{—}1_) was performed with an ABI 7900 HT. The [T>C] polymorphism is a non-synonymous coding SNP resulting in an amino acid change from Serine to Proline at position 185 in the AGXT2L1 protein. This change in amino acid from a polar to a non-polar side chain is predicted to result in a change in the protein function of AGXT2L1. The heterozygosity of the SNP for European, Asian, and subSaharan African populations was 0.15, 0.55, and 0.45 respectively according to NCBI dbSNP 36.1. The SMRI identifiers for the samples show black (1), native American (1), Hispanic (1), and white (102). The 102 samples identified as white were used for calculations of preliminary association with phenotype as each ethnic group represented varying heterozygosity.

### Results

Demographics

The demographics for all subjects and statistical summaries of the 88 subjects analysed (Table 4) showed no significant differences between comparisons of patient to control groups for RNA quality (28S/18S), and age (all p>0.05). As described in methods brain pH was decreased in both the schizophrenia and bipolar disorder groups compared to control groups (p<0.05), and the refrigeration interval was significantly increased in both psychiatric groups compared with controls (p<0.05). The gender was not equally balanced in the BPD and control group, with a trend for increase in the number of females in the BPD group (Pearson's Chi-Square=3.61, p=0.057). Age, brain pH, and gender have been shown previously to be significant variables in microarray studies (Galfalvyet al. (2003) BMC Bioinformatics 4(1): 37; Vawter et al. (2004) Neuropsychopharmacology 29(2): 373-384; Altar et al. (2005) Biol Psychiatry 58(2): 85-96; Erraji-Benchekroun et al. (2005) Biol Psychiatry 57(5): 549-558; Iwamoto et al. (2005) Hum. Mol. Genet., 14(2): 241-253; Vawter et al. (2006) Mol. Psychiatry. 11(7): 663-679) and were included in all ANCOVAs. PMI, refrigeration interval, and RNA quality, were entered as covariates for a final polish of gene expression in ANCOVA. The histograms of p-values for each demographic variable of pH, age, PMI, RNA quality, refrigeration interval demonstrated the validity of selecting pH and age as primary covariates along with gender and diagnosis as main effects. PMI, RNA quality, and refrigeration interval (RI) were not strong contributors to gene expression, nevertheless these covariates were used in the final analysis since the groups were not balanced well for PMI and RI (Table 4).

Outlier Chips

Each chip was evaluated in reference to the following criteria: principal components analysis, slope of positive control probes, number of genes that were flagged as good quality, number of genes with negative number for expression (undetected), and average correlation index. There were eleven outlier chips eliminated, a summary of chips meeting the outlier criteria is shown in Table 4. 88 microarrays were used in the remainder of the analyses. Six samples had low cRNA and were not hybridized to microarrays (Table 4).

The final sample size was a total of 88 subjects: SZ (32), BPD (27), and controls (29). The demographics showed significant differences in pH, PMI, and the refrigeration interval between controls and bipolar disorder. There were also significant differences for pH and refrigeration interval between schizophrenia and controls (Table 4). These variables were used as covariates.

Unrestricted Analysis of Subjects By ANCOVA

The first method chosen was ANCOVA with unrestricted pH, followed with a second ANCOVA with subjects restricted to pH above the median to determine whether effects might be seen in subjects that did not show the lowest pH in the study.

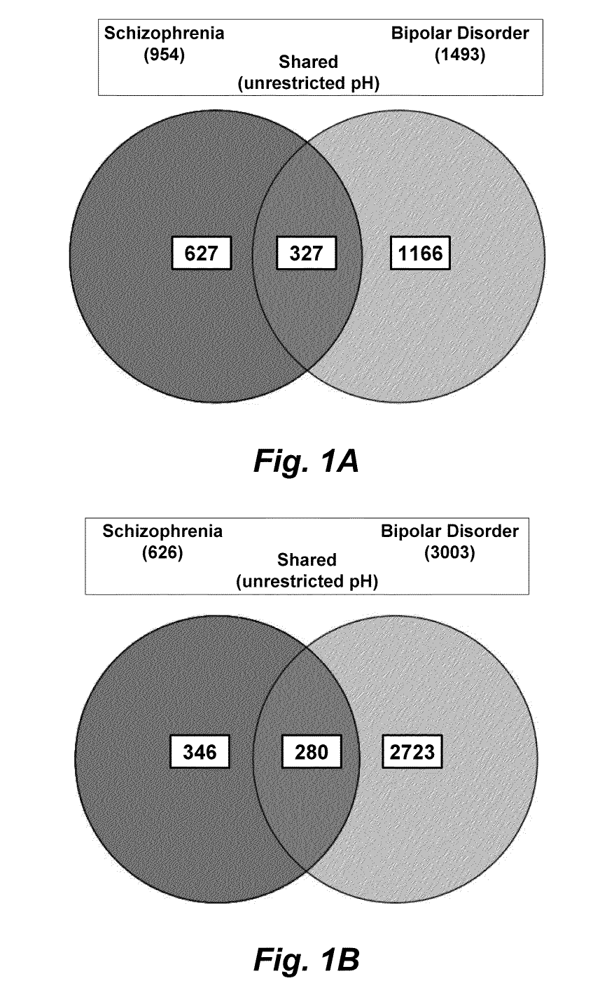

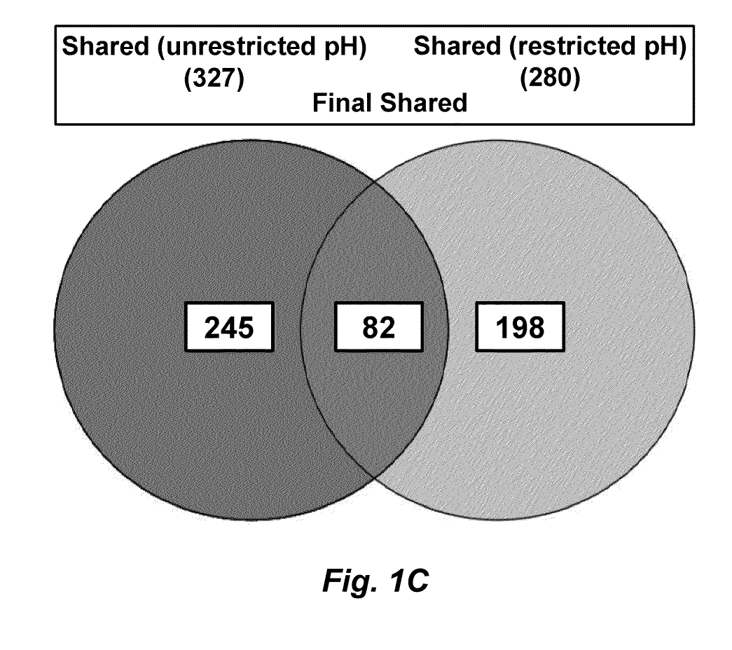

The molecular profile involving schizophrenia and bipolar disorder showed overlap of 327 differentially expressed genes in DLPFC following ANCOVA with covariates of pH and age (FIG. 1A). There were 1,793 dysregulated genes not shared in both disorders with a criterion of (p<0.05, FIG. 1A). The total number of genes shared in both bipolar disorder and schizophrenia appeared larger compared to the expected number by chance, the ratio of observed/expected was 4.57 which suggested an enrichment of shared genes (Table 5). This analysis used a common control group as a reference, which could inflate the number of shared genes in bipolar and schizophrenia since the analysis of psychiatric group differences was not independent and gene expression among significantly differentially expressed genes can be correlated (see AGXT2L1 correlation).

TABLE 5
The analysis of both BPD and SZ were conducted with ANCOVA
using diagnosis and gender as main effects with age and pH as covariates.
The number of significant genes in each ANCOVA without
restriction of subjects by pH, and after restriction to subjects with
pH > 6.57 shows an increased number of shared genes beyond
expected chance levels for BPD and SZ. The relationship was tested
in a 2 × 2 chi-square analysis of the cells shown in gray.

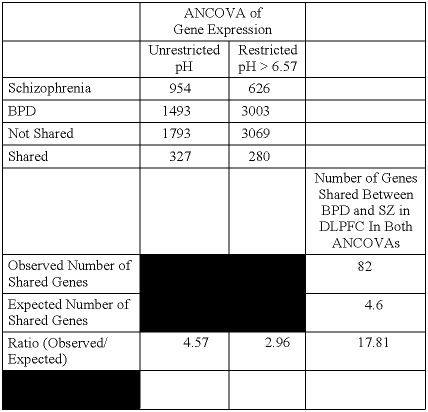

Restricted Analysis of Subjects By ANCOVA

A second ANCOVA was restricted to subjects in each group with pH above the median pH of 6.57 and with the same covariates of pH and age. The molecular profile involving schizophrenia and bipolar disorder showed overlap of 280 differentially expressed genes in DLPFC following ANCOVA. There were 3,069 dysregulated genes (p<0.05) not shared for both disorders (FIG. 1B) about 1.7 more genes than in the unrestricted pH analysis.

The number of genes shared in bipolar disorder and schizophrenia also suggested an enrichment of shared genes, i.e. the observed/expected ratio was 2.96 (Table 5). A similar caveat as in the unrestricted analysis is applicable, i.e. a common control group was used as a reference, which could inflate the number of shared genes.

Combining Both ANCOVA Results

After combining both ANCOVA gene lists (Venn diagrams FIG. 1A and FIG. 1B), there were 82 significant genes shared between BPD and schizophrenia (FIG. 1C, Table 6), only 5 genes were expected by chance and the chi-square was highly significant for 82 genes found (Table 5, p=0.013). The entire list of 82 genes (Table 6) was further subjected to a final demographic polish by ANCOVA with PMI, RI, RNA quality as well as age and pH as simultaneous covariates, and 71 genes passed 3 ANCOVA filters for the final demographic analysis . Genes that passed all 3 ANCOVA filters are shown without asterisk, and genes that did not survive all 3 analyses have an asterisk (Table 6).

Brain Relevance of Gene Expression

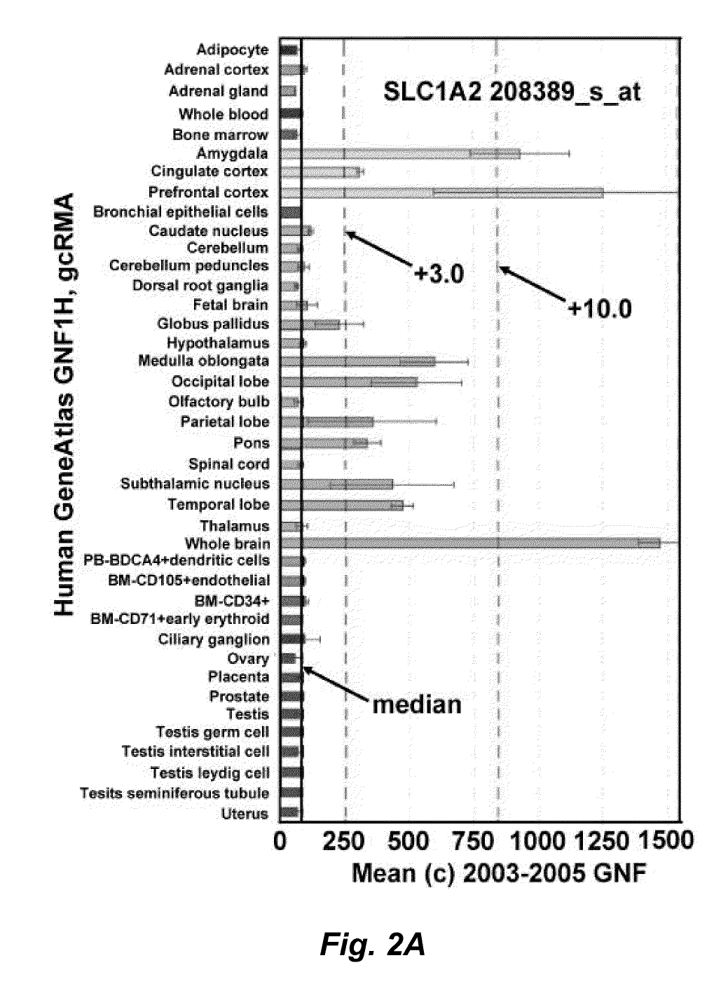

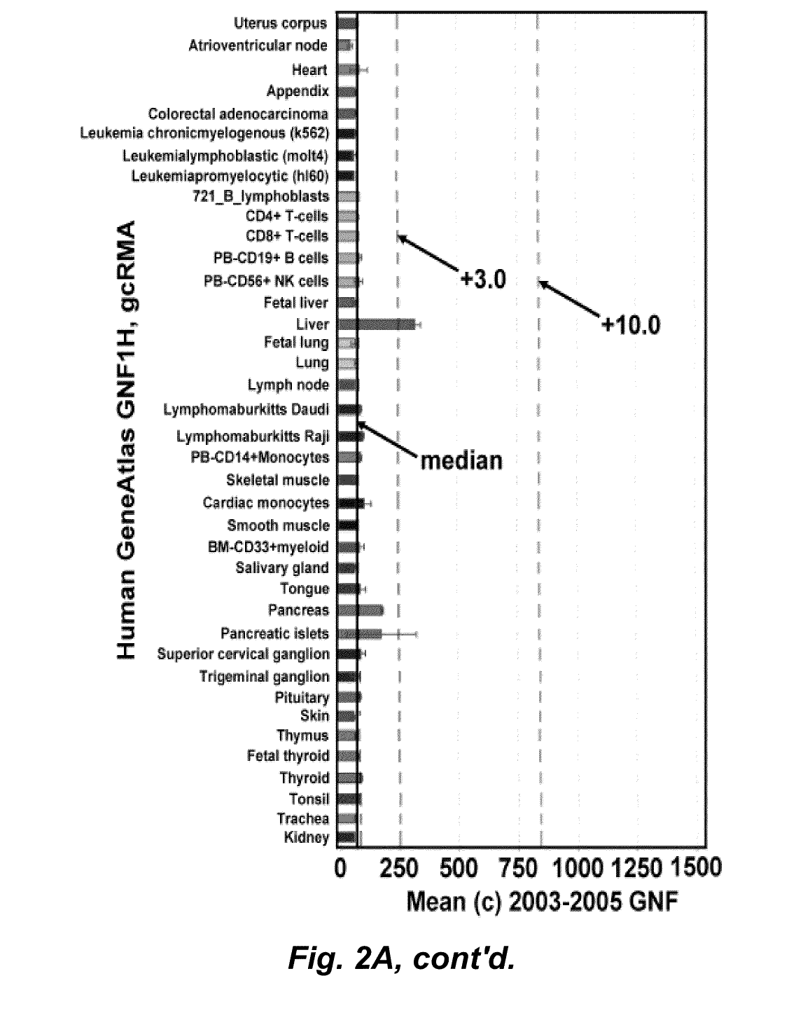

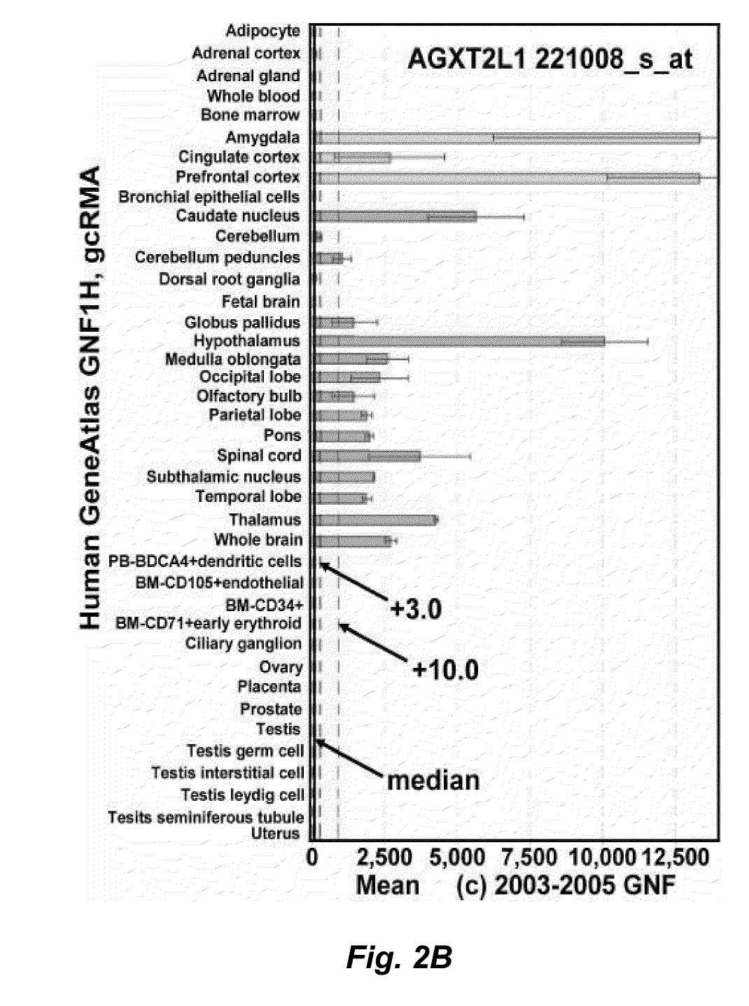

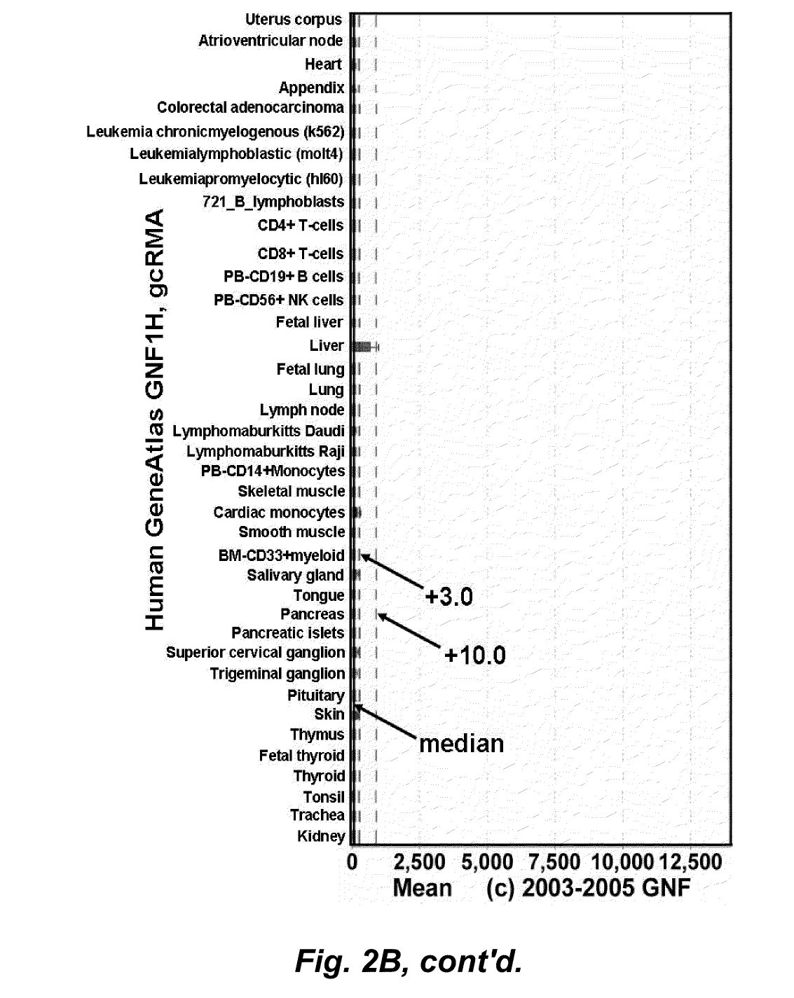

The list of 82 genes developed for shared differential expression was next queried at Novartis Gene Symbol Atlas for brain expression levels (Su et al. (2004) Proc. Natl. Acad. Sci., USA, 101(16): 6062-6067). It was determined that 3 genes (AGXT2L1, SLC1A2, shown in FIG. 2, and TU3A) have expression restricted to brain regions, and were not expressed or showed baseline levels in samples from 56 tissues or cell lines. The following significant genes were listed (Table 6) as greater than 10× median expression in one or more brain regions which was another indicator of brain enrichment (Id.): TU3A, AGXT2L1, TUBB2B, SLC1A2, SOX9, ATP6V1H, GMPR, EMX2, AHNAK, and IMPA2.

Since AGXT2L1 (alanine-glyoxylate aminotransferase 2-like 1) gene appeared to be restricted to brain expression and was strongly dysregulated in both BPD and SZ, evidence of coregulation with AGXT2L1 (Pearson Correlation p-value<0.25×10⁻⁶) was found for 50 genes. The p-value for correlations of AGXT2L1with the final gene list is shown following Bonferroni correction for all 19,980 discovery genes on the chip analysed (Table 6). It was noticeable that genes with low correlation with AGXT2L1 and/or with low expression values were especially vulnerable to effects of the final demographic analysis (Table 6). Thus, in the final list of significant genes there were 10 genes highly enriched in brain, and 3 genes showed restricted expression to the brain.

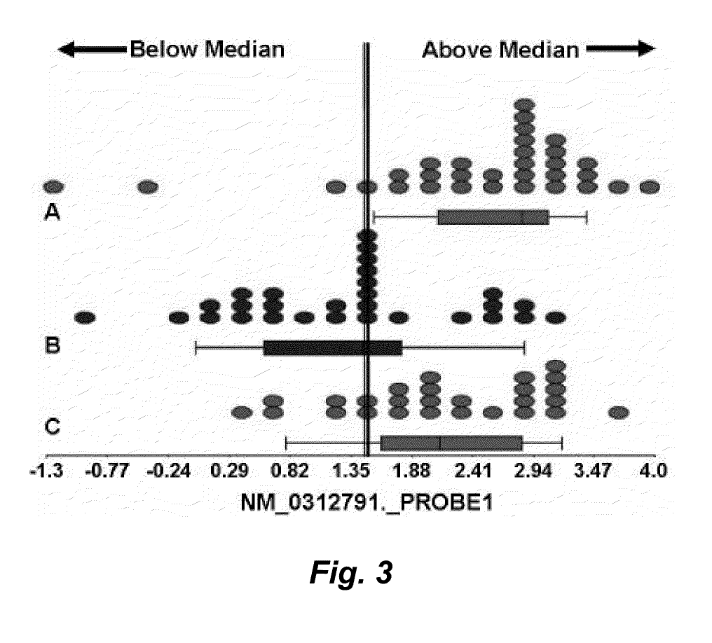

The distribution of AGXT2L1 expression in BPD, SZ, and controls was examined (FIG. 3). The differences in AGXT2L1 levels in bipolar disorder and schizophrenia were not due to a few extreme outliers (FIG. 3). Individuals with psychiatric disorder (48/9) showed above the median of control's AGXT2L1 expression levels (FIG. 3) and this distribution was highly significant (Fisher's Exact Test, p =0.000001). An odds ratio of 11.4 for developing a psychiatric disorder based upon above median expression of AGXT2L1 was calculated. The distributions of AGXT2L1 across all 3 groups appeared to have two modes, one at 3 (median centered units) and the second at 1.5 (median centered units), suggesting a genetic component in regulation. This hypothesis was tested by genotyping one SNP, since the entire gene consisted of 1 LD block for all SNPs in the CEPH European sample.

Genotyping Results

The nonsynonymous coding SNP rs1377210 for AGXT2L1 [T>C] polymorphism results in an amino acid Ser>Pro at residue 185 and likely change of protein function due to amino acid change from a polar to non-charged side chain. The genotype and allelic ratio for caucasian subjects (Table 7) was tested in a preliminary allelic association for the nonsynonymous AGXT2L1. The association was not significant in either BPD (Fishers Exact test p=0.06) or schizophrenia (p=0.14) compared with controls (Table 7). A combined analysis with SZ and BPD compared to controls was also at a trend level (p=0.08). These trends require a larger number of total subjects (1,000-1,500) for an 80%-95% power for detecting an association with AGXT2L1 SNP rs1377210.

Pathway Analysis

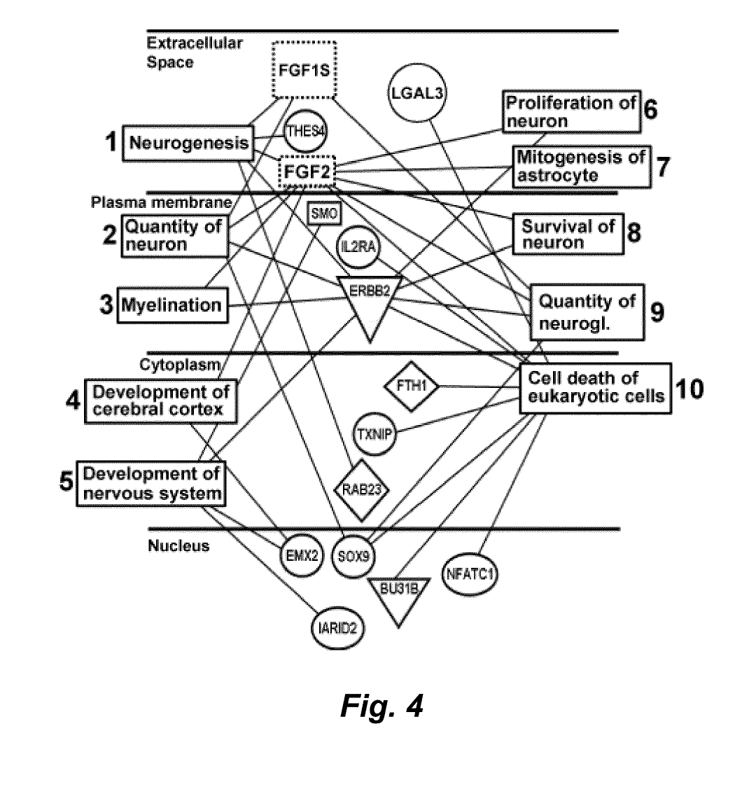

The 82 genes initially found significant for both SZ and BPD (Table 6) were entered into Ingenuity Pathway Analysis (IPA v 4.0) and EASE for evaluating potential pathways that were significantly dysregulated. The most significant functional category (p=3.19×10⁻¹⁹) was cellular growth and proliferation that contained the following genes: BUB1B, EMX2, ERBB2, FGF2, FTH1, IL2RA, LGALS3, MAFG, NFATC1, PVR, RERG, SMCY, SMO, SOX9, TXNIP (FIG. 4). There were 85 high level functional categories and this was the top high level category.

TABLE 6
Genes that pass two ANCOVAs (with and without restriction of pH)
for Diagnosis, Gender, pH and age and show significant group differences for both bipolar
disorder and schizophrenia. The results were significant for each gene and both disorders
following adjustment of means by regression for gender, pH, and age. The pattern of
AGXT2L1 expression is exclusively brain (Su et al. (2004) Proc. Natl. Acad. Sci., USA,
101(16): 6062-6067), and other genes that display high correlations suggests similar patterns of
regulation. The Pearson correlation of each gene expression with AGXT2L1 as a reference
gene is shown following Bonferroni correction for all genes analyzed. The list is sorted by p-
value of correlated expression between each gene and AGXT2L1. There were 82 genes that
passed both ANCOVAs shown in this table. Underlined genes showed a relatively high brain
expression compared with other tissues, i.e. 10 times median expression in brain. A “fold
change” greater than 1 indicates upregulation (increased expression) of the gene, while a “fold
change” less than 1 indicates downregulation (decreased expression) of the gene.

| | | | | | | p-value | |
|---|---|---|---|---|---|---|---|
| | | | | | | Pearson | |
| | | | | | | correlation | |
| | | | | | Fold | with | |
| | | | Fold | p-value | Change | AGXT2L1 | |
| Gene Symbol | Accession No. | p-value | Change | Bipolar | Bipolar | (Bonferroni | Median |
| (ENTREZ) | NCBI | Schizoprenia | Schiz. | Disorder | Disorder | Correction) | Expression |
| AGXT2L1 | NM_031279 | 4.65E−04 | 2.15 | 1.14E−02 | 1.72 | | 1.5 |
| SLC14A1 | NM_015865 | 1.93E−04 | 2.81 | 2.59E−02 | 1.82 | 2.27E−21 | −1.6 |
| EMX2 | NM_004098 | 5.37E−04 | 1.61 | 3.28E−03 | 1.49 | 8.93E−20 | 1.5 |
| SLC2A10 | NM_030777 | 3.07E−02 | 1.38 | 2.80E−02 | 1.39 | 2.85E−18 | −0.3 |
| DKFZp434C0328 | NM_017577 | 3.06E−04 | 1.54 | 1.21E−02 | 1.34 | 2.95E−18 | −0.6 |
| PARD3 | AF196185 | 1.26E−03 | 1.44 | 3.99E−03 | 1.38 | 6.83E−17 | 0.5 |
| RERG | NM_032918 | 3.41E−02 | 1.34 | 3.30E−02 | 1.34 | 7.70E−17 | 1.9 |
| IL17RB | NM_172234 | 3.79E−04 | 1.61 | 1.77E−03 | 1.52 | 1.04E−16 | 0.8 |
| SSPN | NM_005086 | 3.40E−03 | 1.43 | 2.68E−02 | 1.31 | 1.88E−16 | 1.4 |
| ADHFE1 | NM_144650 | 1.14E−03 | 1.49 | 2.62E−02 | 1.31 | 8.19E−16 | 1.5 |
| MMP28 | NM_032950 | 3.12E−03 | 1.53 | 3.00E−02 | 1.36 | 8.27E−16 | −1.1 |
| LRRC16 | NM_017640 | 6.00E−03 | 1.26 | 1.85E−03 | 1.31 | 1.06E−14 | 0.3 |
| SOX9 | NM_000346 | 6.63E−04 | 1.70 | 1.74E−03 | 1.62 | 1.40E−14 | 2.2 |
| GNG12 | NM_018841 | 1.54E−03 | 1.36 | 4.46E−04 | 1.41 | 1.14E−13 | 0.3 |
| MGST1 | NM_020300 | 1.12E−03 | 1.52 | 5.67E−03 | 1.43 | 2.08E−13 | 3.3 |
| NOPE | NM_020962 | 2.02E−04 | 1.49 | 7.86E−03 | 1.32 | 5.98E−13 | 0.7 |
| TU3A | NM_007177 | 4.78E−03 | 1.35 | 7.25E−03 | 1.33 | 6.06E−13 | 5.0 |
| PCTP | NM_021213 | 1.95E−03 | 1.25 | 5.51E−03 | 1.22 | 1.81E−12 | 0.6 |
| HIF3A | NM_022462 | 2.79E−04 | 1.95 | 4.10E−02 | 1.44 | 2.06E−12 | 0.0 |
| NOTCH2NL | AK022008 | 2.56E−02 | 1.28 | 9.79E−03 | 1.34 | 3.31E−12 | 2.0 |
| SMO | NM_005631 | 7.85E−03 | 1.36 | 1.01E−03 | 1.46 | 9.61E−12 | −1.2 |
| RAB31 | NM_006868 | 4.04E−04 | 1.33 | 5.03E−05 | 1.39 | 1.30E−11 | 1.9 |
| ALDH7A1 | AK021800 | 4.22E−03 | 1.41 | 6.57E−03 | 1.38 | 1.36E−11 | 0.8 |
| TXNIP | NM_006472 | 9.00E−03 | 1.47 | 1.11E−02 | 1.46 | 3.82E−11 | 2.2 |
| PPARA | AB073605 | 3.64E−02 | 1.25 | 3.56E−02 | 1.25 | 4.04E−11 | 0.0 |
| FGF2 | NM_002006 | 1.47E−02 | 1.46 | 1.96E−02 | 1.43 | 4.94E−11 | 0.1 |
| NFATC1 | NM_172388 | 1.19E−03 | 1.64 | 3.07E−03 | 1.57 | 5.07E−11 | −2.2 |
| RAB34 | NM_031934 | 5.85E−03 | 1.45 | 2.01E−02 | 1.36 | 5.77E−11 | 1.0 |
| RAB34 | NM_031934 | 1.52E−02 | 1.38 | 3.38E−02 | 1.32 | 5.39E−10 | 2.3 |
| TUBB2B | NM_178012 | 2.39E−03 | 1.48 | 1.58E−02 | 1.36 | 5.59E−10 | 3.9 |
| MCCC2 | C00869 | 9.56E−04 | 1.33 | 1.73E−02 | 1.22 | 1.27E−09 | 2.4 |
| C14orf128 | NM_032751 | 1.63E−02 | 1.23 | 1.53E−02 | 1.23 | 2.74E−09 | −0.1 |
| ERBB2 | NM_001005862 | 2.64E−03 | 1.43 | 2.48E−02 | 1.30 | 2.10E−08 | 0.1 |
| IMPA2 | NM_014214 | 1.86E−02 | 1.28 | 3.55E−02 | 1.24 | 4.57E−08 | −1.5 |
| UNG | NM_080911 | 1.13E−02 | 1.21 | 2.53E−02 | 1.19 | 7.27E−08 | 1.5 |
| SLC1A2 | BU662414 | 2.58E−02 | 1.54 | 4.57E−02 | 1.47 | 7.43E−08 | −1.2 |
| MGST1 | AI823969 | 2.72E−03 | 1.74 | 1.91E−03 | 1.78 | 1.03E−07 | −1.0 |
| FMO5 | NM_001461 | 6.17E−03 | 1.37 | 1.06E−02 | 1.34 | 1.11E−07 | −2.5 |
| C6orf4 | NM_147200 | 6.00E−03 | 1.37 | 2.48E−02 | 1.29 | 1.97E−07 | −0.8 |
| LGALS3 | NM_002306 | 1.41E−03 | 1.45 | 3.30E−02 | 1.27 | 2.78E−07 | 1.4 |
| NDP52 | NM_005831 | 9.32E−03 | 1.27 | 4.29E−02 | 1.21 | 1.19E−06 | 1.4 |
| FTH1* | NM_002032 | 1.58E−03 | 1.31 | 4.08E−02 | 1.19 | 4.51E−06 | 4.0 |
| MT1X | NM_005952 | 4.80E−04 | 1.83 | 1.22E−02 | 1.53 | 4.63E−06 | 4.3 |
| ZC3HAV1 | NM_020119 | 2.05E−03 | 1.37 | 4.27E−03 | 1.34 | 5.98E−06 | 1.1 |
| OGDH | BE348404 | 2.02E−02 | 0.81 | 6.63E−03 | 0.79 | 6.82E−06 | 0.6 |
| ZNF254 | NM_004876 | 6.40E−03 | 1.24 | 9.93E−03 | 1.22 | 2.27E−05 | −0.5 |
| FLJ10970 | NM_018286 | 3.12E−02 | 1.39 | 1.38E−02 | 1.45 | 3.26E−04 | −0.9 |
| LIX1 | N99205 | 3.56E−02 | 1.30 | 6.79E−03 | 1.41 | 3.46E−04 | −2.9 |
| LOC283537 | NM_181785 | 1.70E−02 | 1.17 | 1.17E−02 | 1.19 | 3.98E−04 | 0.8 |
| C14orf135 | NM_022495 | 3.79E−02 | 1.18 | 7.90E−03 | 1.23 | 4.92E−04 | 0.7 |
| TCTEL1 | NM_006519 | 7.51E−03 | 1.27 | 3.88E−02 | 1.20 | 1.59E−03 | 2.9 |
| ZMYND12 | NM_032257 | 7.33E−03 | 1.28 | 5.75E−03 | 1.29 | 4.76E−03 | −0.8 |
| NMU | NM_006681 | 4.41E−03 | 0.58 | 3.01E−02 | 0.66 | 6.16E−03 | −0.9 |
| THBS4 | NM_003248 | 7.97E−03 | 1.24 | 1.56E−02 | 1.21 | 1.16E−02 | 0.9 |
| ZNF261 | NM_005096 | 2.68E−02 | 1.17 | 4.45E−02 | 1.15 | 1.57E−02 | 2.3 |
| FLJ21148 | NM_024860 | 1.22E−02 | 0.82 | 2.17E−02 | 0.83 | 2.20E−02 | −1.9 |
| JARID2 | NM_004973 | 1.76E−02 | 1.23 | 1.15E−02 | 1.24 | 3.16E−02 | 1.4 |
| GMPR | M24470 | 3.77E−02 | 1.22 | 5.24E−03 | 1.31 | 1.68E−01 | −0.9 |
| FLJ10496 | NM_018114 | 4.21E−02 | 0.88 | 3.56E−02 | 0.88 | 1.79E−01 | 0.9 |
| AHNAK | AL047960 | 3.39E−02 | 1.38 | 1.64E−02 | 1.44 | 2.11E−01 | 0.1 |
| MAFG | NM_002359 | 9.05E−03 | 0.85 | 1.53E−03 | 0.82 | 2.20E−01 | 1.0 |
| ZNF442 | NM_030824 | 4.89E−04 | 1.80 | 3.72E−04 | 1.82 | 2.59E−01 | −2.3 |
| ATP6V1H | NM_213620 | 1.51E−03 | 0.79 | 3.70E−02 | 0.86 | 3.27E−01 | 2.3 |
| HEBP2 | NM_014320 | 1.73E−03 | 1.31 | 2.34E−02 | 1.21 | 6.66E−01 | 0.0 |
| KIAA0515* | BX647842 | 2.90E−02 | 1.17 | 2.10E−02 | 1.19 | 8.95E−01 | 1.5 |
| UBXD3 | BX648631 | 2.14E−02 | 1.19 | 1.92E−02 | 1.20 | 1.00E+00 | 0.0 |
| KIAA0020* | NM_014878 | 9.20E−03 | 0.45 | 4.41E−02 | 0.54 | 1.00E+00 | −3.0 |
| Transcribed locus | BM701748 | 2.61E−02 | 1.49 | 7.34E−03 | 1.63 | 1.00E+00 | −0.1 |
| PBX4 | NM_025245 | 7.01E−03 | 1.29 | 6.26E−04 | 1.39 | 1.00E+00 | −0.4 |
| CACNB1 | NM_199247 | 1.43E−02 | 0.81 | 2.90E−03 | 0.77 | 1.00E+00 | 0.2 |
| PVR | M24407 | 1.91E−02 | 0.82 | 2.18E−04 | 0.73 | 1.00E+00 | 1.7 |
| ZNF268 | NM_152943 | 1.16E−02 | 1.22 | 6.86E−03 | 1.23 | 1.00E+00 | 0.5 |
| CFC1* | AW139377 | 3.04E−03 | 2.33 | 3.03E−03 | 2.33 | 1.00E+00 | −5.6 |
| RSC1A1 | NM_006511 | 1.35E−02 | 1.31 | 7.05E−04 | 1.46 | 1.00E+00 | −0.7 |
| MDH1 | NM_005917 | 4.76E−02 | 0.84 | 1.32E−03 | 0.75 | 1.00E+00 | 5.5 |
| ZNF599 | H45564 | 2.11E−04 | 2.27 | 3.84E−04 | 2.19 | 1.00E+00 | −3.5 |
| SMCY* | NM_004653 | 1.79E−02 | 0.69 | 4.10E−03 | 0.63 | 1.00E+00 | 3.0 |
| RAB23* | NM_183227 | 4.97E−02 | 1.18 | 1.24E−02 | 1.23 | 1.00E+00 | 0.6 |
| BUB1B* | NM_001211 | 2.19E−03 | 3.17 | 9.82E−03 | 2.53 | 1.00E+00 | −5.5 |
| IL2RA* | NM_000417 | 2.30E−02 | 1.81 | 3.09E−02 | 1.77 | 1.00E+00 | −5.1 |

*The following genes were sensitive to PMI: FTH1, KIAA0515, KIAA0020, CFC1, SMCY, RAB23, BUB1B, IL2RA. Only FTH1 showed a significant correlation with AGXT2L1. Thus, these genes showed non significant differential expression (p > 0.05) in both BPD and schizophrenia when PMI was run as a covariate.

Genes from two subcategories ‘Nervous System Development and Function’, and ‘Cell Death’ were subsets of Cellular Growth and Proliferation. The Nervous System Development and Function category (quantity of neuroglia, quantity of neurons, neurogenesis, development of nervous system) genes dysregulated in both BPD and SZ were THBS4, SOX9, EMX2, RAB2B, JARID2, FGF2, ERBB2, SMO. These genes were significant over-represented in this category (Fisher's Exact test p=8.34×10⁻⁰⁶). A second functional category, Cell Death (p-value=2.98×10⁻⁰⁸) contained the following genes: BUB1B, ERBB2, FGF2, FTH1, IL2RA, LGALS3, NFATC1, SOX9, TXNIP that were dysregulated in both BPD and schizophrenia (FIG. 4).

The two functional categories (Neurogenesis, Cell Death) shared genes and one example (FIG. 4) is ERBB2 (a receptor for neuregulin) which is an important schizophrenia/bipolar disorder susceptibility gene that was dysregulated in DLPFC.

Q-PCR

The candidate gene list was assayed by QPCR that consisted of genes significantly dysregulated in both BPD and SZ. The overall concordance for significant genes on both microarray and Q-PCR platforms was 58% (Table 7). The housekeeping gene used for nomalization was CRSP9. In bipolar disorder all genes were validated by QPCR (Table 7). All genes tested for schizophrenia showed the appropriate fold change concordance however SLC1A2, SLC6A8, TU3A, and GLUL were not significant (p<0.25). Four genes that were significantly dysregulated by microarray in both bipolar disorder and schizophrenia however were not validated by Q-PCR testing in either disorder: HOMER1, MCCC2, CORT, RGS4. These four genes while significant by microarray for both disorders, may be false positives for microarray or false negatives with the SybrGreen method. The correlations of fold change for QPCR and microarray platform for the 17 genes tested showed a correlation coefficient for bipolar disorder of 0.61 and for schizophrenia of 0.61.

TABLE 7
Q-PCR results for validation of selected genes. The overall
concordance for significant genes on both microarray and Q-PCR platforms was 58%. The list
is sorted by gene symbol, all genes showed significant gene expression differences in both
schizophrenia and bipolar disorder compared to controls by microarray. In bipolar disorder all
genes were validated by QPCR listed in the table, and for all genes for schizophrenia except
SLC1A2, SLC6A8, and GLUL. The housekeeping gene used for normalization was CRSP9.
The following genes that were significantly dysregulated by microarray in both bipolar
disorder and schizophrenia were not validated by Q-PCR in either disorder: HOMER1,
MCCC2, CORT, RGS4 (data not shown). A “fold change” greater than 1 indicates
upregulation (increased expression) of the gene, while a “fold change” less than 1 indicates
downregulation (decreased expression) of the gene.

| | Schizophrenia | | | | Bipolar Disorder | | | |
|---|---|---|---|---|---|---|---|---|
| Gene | Microarray | Fold | PCR | Fold | Microarray | Fold | Microarray | Fold |
| Symbol | p-value | Change | p-value | Change | p-value | Change | p-value | Change |
| AGXT2L1 | 8.38E−05 | 2.22 | 1.19E−02 | 2.31 | 1.32E−03 | 1.82 | 2.94E−03 | 2.99 |
| CASP6 | 1.56E−02 | 1.19 | 7.00E−03 | 2.76 | 1.62E−02 | 1.23 | 1.11E−02 | 2.55 |
| EPHB4 | 2.63E−05 | 1.45 | 4.48E−02 | 1.96 | 1.03E−02 | 1.28 | 4.69E−02 | 2.11 |
| GLUL | 5.97E−05 | 1.50 | 2.51E−01 | 1.51 | 1.81E−03 | 1.40 | 2.26E−03 | 2.76 |
| HMGB2 | 2.05E−05 | 1.39 | 5.22E−02 | 1.6 | 4.37E−04 | 1.40 | 5.33E−04 | 3.52 |
| MAOA | 7.92E−03 | 1.22 | 3.23E−02 | 2.54 | 8.28E−04 | 1.33 | 2.08E−02 | 3.29 |
| NOTCH2 | 2.12E−02 | 1.44 | 1.92E−02 | 2.19 | 5.58E−03 | 1.52 | 5.11E−02 | 1.89 |
| SLC1A2 | 8.13E−03 | 1.41 | 2.47E−01 | 1.51 | 3.06E−03 | 1.48 | 4.63E−03 | 2.66 |
| SLC1A3 | 1.32E−04 | 1.85 | 3.02E−02 | 2.22 | 4.83E−03 | 1.60 | 7.25E−04 | 3.73 |
| SLC6A8 | 1.84E−03 | 1.30 | 5.75E−02 | 2.26 | 1.68E−02 | 1.21 | 1.94E−02 | 3.08 |
| TNFSF10 | 3.33E−05 | 0.46 | 1.38E−02 | 0.36 | 1.90E−03 | 0.43 | 2.81E−02 | 0.34 |
| TNFSF8 | 6.90E−04 | 2.91 | 3.63E−02 | 2.77 | 4.42E−04 | 2.87 | 3.94E−02 | 3.28 |
| TU3A | 4.78E−03 | 1.35 | 6.83E−02 | 1.83 | 7.25E−03 | 1.33 | 6.00E−03 | 2.62 |

Cross-Validation of Results

The initial discriminant analysis correctly identified 100% of each group membership (Table 8). This classification result was expected because genes were chosen based on the entire sample to discriminate controls from both BPD and SZ. However, in a more statistically rigorous analysis, an 11×4 two-level cross nested validation approach was used with the top 50 genes selected from Table 6. The two-level cross nested validation model correctly assigned BPD and SZ to psychiatric group membership at 79.8% (Table 8).

TABLE 8
Nested model cross validation discriminant analysis for schizophrenia
and bipolar disorder from controls. There were 82 significant genes that
were shared between BPD and schizophrenia that survived both pH
analysis >6.57 and all subjects analysis. There were 50 genes that
showed strong evidence of coregulation with AGXT2L1 (p-value <10-6)
in Table 6. This list of genes was then subjected to a discriminant analysis.
This list of genes completely discriminated BPD and SZ from controls,
and in cross validation with an average of 79.8% across models.
Classification Summary of the Model

| Variable to Predict | Psychiatric |
|---|---|
| # of Predictor Candidates | 50 |
| # of Samples | 88 |
| # of Models | 10 |
| Random Seed | 10001 |
| Presentation Order | Randomly reorder data |
| Model Selection Criterion | Normalized Correct Rate |
| Cross-Validation | 2-Level Nested |
| Outer Partitions | 11 |
| Inner Partitions | 4 |

### Discussion

A comparison of expression profiles in both BPD and SZ produced a list of candidate genes differentially expressed for both disorders and a large set of genes dysregulated in only one disorder. Selective QPCR validation of genes dysregulated in both disorders suggests that this list is a reasonable starting point for common risk factor assessment by gene expression study in another cohort. This list also represents candidate genes some that are brain enriched which might contribute to common pathophysiological mechanisms, and perhaps respond to treatments that are developed in these critical pathways. Intervention in these critical gene expression pathways that are enriched for neurogenesis and cell death (examples of significant pathways) might also lead to an amelioration of symptoms or relief for individuals at high risk, and reduce progression commonly associated with both disorders.

An example of a brain enriched gene, AGXT2L1 (alanine: glyoxylate aminotransferase 2 homolog 1, splice form 1, chr 4q25), showed a significant increase in the number of psychiatric cases demonstrating above median level expression of the AGXT2L1 gene. This gene with a functional coding mutation might be a risk factor for serious lifelong psychiatric illness, however the current function in humans is not known. The preliminary trend for association of the homozygous functional SNP for AGXT2L1 for either bipolar or schizophrenia or both requires a larger association sample for confirming or discarding association in either the coding region or promoter region of this gene. A SAGE study of gene expression showed the AGXT2L1 gene was found in only brain relevant libraries (CGAP libraries) confirming the SymAtlas query. According to the NCBI conserved domain database there are predicted 4 domains present in AGXT2L1: 1) GabT, 4-aminobutyrate aminotransferase and related aminotransferases which involves amino acid transport and metabolism; 2) BioA, Adenosylmethionine-8-amino-7-oxononanoate aminotransferase involves coenzyme metabolism; 3) ArgD, Ornithine/acetylornithine aminotransferase which involves amino acid transport and metabolism; and 4) HemL, Glutamate-1-semialdehyde aminotransferase which involves coenzyme metabolism.

Another exciting finding of the present study is in the cellular growth and proliferation pathway. It was suggested that ERBB2 receptor blockage with the monoclonal antibody trastuzumab would be a beneficial treatment for schizophrenia (Sastry and Ratna (2004) Medical Hypotheses 62(4): 542-545). The blocking of ERBB2 receptor is based on a possible decrease in neuregulin activation of the receptor which could alter synaptic plasticity. This type of intrathecal therapy of trastuzumab to improve synaptic plasticity would need to be further demonstrated in animal models (Nawa and Takei (2006) Neurosci Res., 56(1): 2-13; O'Tuathaigh et al. (2006) Neurosci Biobehav Rev. June 16 [Epub ahead of print]). Association of the neuregulin-ERBB receptor signaling alterations continues to be an important candidate pathway in the forefront of research into the pathophysiology of schizophrenia (Stefansson et al. (2002) Am. J. Human Genetics 71(4): 877-892; Stefansson et al. (2003) Am. J. Human Genetics 72(1): 83-87; Stefansson et al. (2004) Annals Med. 36(1): 62-71; Stefansson et al. (2003) Molecular Psychiatry 8(7): 639-640; Law et al. (2006) Proc. Natl. Acad. Sci., USA, 103(17): 6747-6752). Another member of the pathway, SOX9 was dysregulated, and the SOX family is important in neurodevelopment (Wegner and Stolt (2005) Trends Neurosci., 28(11): 583-588) and in particular SOX9 can convert cells in neurogenic lineage to gliogenic lineage. Thus two examples of genes in the cellular growth and proliferation functional category that were dysregulated in BPD and SZ represent important future targets for modulation and association with genetic variation. The FGF family is another clear example of genes involved in neurogenesis that were dysregulated in the present study and in other studies of mood disorders (Evans et al. (2004) Proc. Natl. Acad. Sci., USA, 101(43): 15506-15511; Gaughran et al. (2006) Brain Res. Bull., 70(3): 221-227; Turner et al. (2006) Biological Psychiatry 59(12):1128-1135).

The profile of both SZ and BPD tested whether shared vulnerability genes appeared to be more numerous than non-shared genes. The preliminary answer from this study shows a far greater proportion of nonshared to shared genes. The shared genes formed a fraction, 82 genes, compared with results from the same combined ANCOVA analyses there were about 443 genes that were dysregulated in either BPD (198), or SZ (245). Notably only 5 genes were expected to overlap from combined ANCOVA analyses for BPD and SZ. This core set of 82 genes was enriched by 17 fold might confer susceptibility to both disorders, but could represent important alterations in response to medications administered to both groups (Iwamoto et al. (2005) Hum. Mol. Genet., 14(2): 241-253), or downstream events manifest during a chronic psychiatric illness.

Genes that are highly correlated with AGXT2L1, such as SLC1A2 and TU3A and were enriched in brain merit further study as potential susceptibility factors in BPD and SZ. Alterations to the glutamatergic system in brain have been reported for SLC1A2 (EAAT2 or GLT, high affinity glutamate transporter, predominantly astroglial) expression alterations in psychiatric disorders (Smith et al. (2001) Am. J. Psychiatry 158(9): 1393-1399; McCullumsmith and Meador-Woodruff (2002) Neuropsychopharmacology 26(3): 368-375; Choudary et al. (2005) Proc. Natl. Acad. Sci., USA, 102(43): 15653-15658; Balcar and Nanitsos (2006) Neuropsychopharmacology 31(3): 685-6; author reply 687-688) although not unegivocally demonstrated in all brain regions studied. Caution regarding the isoform specificity that is of pathogenetic importance has been raised since both EAAT2a and EAAT2b are alternatively spliced exons for the same gene SLC1A2 (NM_{—}004171) (Lauriat et al. (2006) Neuroscience 137(3): 843-851). Our primers were located at the 3′ end in the last exon 10, and we targeted EAAT2a in QPCR validation which is the predominant isoform in human brain (Id.). The SymAtlas results (Su et al. (2004) Proc. Natl. Acad. Sci., USA, 101(16): 6062-6067) showed that the SLC1A2 gene is brain enriched, but recent work has shown SLC1A2 expression in peripheral organs (Berger and Hediger (2006) Anat Embryo' (Berl). 211(6):595-606). The first genotype study did show a positive association of schizophrenia to SLC1A2 in Japanese samples (Deng Deng et al. (2004) BMC psychiatry [electronic resource] 4: 21).

The impact of pH sensitive genes was reduced in a stringent three step analysis after controlling for pH by ANCOVA and removing low pH subjects. Other studies e.g. (Konradi et al. (2004) Arch. Gen. Psychiatry 61(3): 300-308; Prabakaran et al. (2004) Mol Psychiatry, 9(7):684-697; Altar et al. (2005) Biol Psychiatry 58(2): 85-96; Sun et al. (2006) J. Psychiatry & Neurosci., 31(3): 189-196) have found that subjects with BPD or SZ have decreased mitochondrial transcript. Although most microarray studies acknowledge that pH will influence mitochondrial gene expression, and when the effect is strongly controlled such as in the present microarray study and others (Iwamoto et al. (2005) Hum. Mol. Genet., 14(2): 241-253; Vawter et al. (2006) Mol. Psychiatry. 11(7): 663-679), the magnitude of mitochondrial gene expression differences in SZ or BPD is markedly reduced. This same effect was seen in the present study, where we found mitochondria genes prior to ANCOVA, and after removing outlier chips and samples, there was a reduction in mitochondrial genes that were significant. Microarrays measure the effect of agonal-pH differences in samples, as the SMR1 samples have a significantly reduced pH, although most cases are rapid deaths. This latter observation has prompted others to regard pH as part of the pathology in BPD and SZ. We find that after careful evaluation with ANCOVA and removal of outlier chips that mitochondrial related transcripts were not over-represented.

Cell death and “neurogenesis’ were over-represented categories in the final list of 82 genes shared for BPD and SZ. Cell death and neurogenesis' categories actually share genes, thus the categories overlap (as shown in FIG. 4). The larger category of Cellular Growth and Proliferation subsumes both subcategories but not entirely. The list of candidate genes involved in Cellular Growth and Proliferation (BUB1B, EMX2, ERBB2, FGF2, FTH1, IL2RA, LGALS3, MAFG, NFATC1, PVR, RERG, SMCY, SMO, SOX9, TXNIP) were developed from a list containing 82 genes in total. We reported an uncorrected p-value (˜10⁻¹⁹), but a conservative correction for 25,000 categories would allow this p-value to survive multiple testing correction.

In conclusion, genes that are shared between both schizophrenia and bipolar disorder merit further consideration in future neurogenomic and cognitive studies especially in vulnerable functional pathways involving cellular proliferation and growth such as neurogenesis, and cell death.

### Example 2

### Identifying Gene Expressions that Differentiates Bipolar Disorder from Schizophrenia

Lymphocyte or dorsolateral prefrontal cortex (DLPFC) RNA patterns were measured using Affymetrix U133 chips or Codelink 20K Bioarrays, respectively. The total number of lymphocyte samples analyzed was: bipolar (n=23), control (n=12), schizophrenia (n=7), and Klinefelters syndrome (n=11). The total number of DLPFC samples analysed were bipolar (n=29), control (n=30), and schizophrenia (n=29).

Specific patterns of gene expression were found that differentiate schizophrenia and bipolar disorder from one another as well as from controls and Klinefelter syndrome. The list of schizophrenia specific genes was also validated in DLPFC, and is presented in Table 9. The list of bipolar specific genes was also validated in DLPFC, and is presented in Table 10.

The biomarker lists in Tables 9 and 10 were also stable for gene expression in three different preparations of blood using fresh lymphocytes, whole blood, or Tempus tube stored blood. Thus, although a specific method might be envisioned for conducting a study in a clinic, the most reliable biomarker genes will not be substantially different across different preparation methods.

TABLE 9
A list of biomarker alterations associated with bipolar disorder only.

| | Affymetrix | | | | | |
|---|---|---|---|---|---|---|
| | Transcript | | | | | |
| | cluster id | | | | | |
| | 3569200 | 3569200 | 3130161 | 2345128 | 2345128 | 3907420 |
| Affymetrix | 2101 01_x_at | 208898_at | 205770_at | 2101 01_x_at | 209091_s_at | 21 6559_x_at |
| Probe Set ID | | | | | | |
| Gene Symbol | ATP6V1D | ATP6V1D | GSR | SH3GLB1 | SH3GLB1 | HNRPA1 |
| Codelink Gene | ATP6V1D | ATP6V1D | GSR | SH3GLB1 | SH3GLB1 | HNRPA1 |
| Symbol | | | | | | |

DLPFC

| | | | | | | |
|---|---|---|---|---|---|---|
| DLPFC p- | 0.1186 | 0.1186 | 0.8244 | 0.7393 | 0.7393 | 0.6077 |
| value (Schiz- | | | | | | |
| Control) | | | | | | |
| LPFC Fold | 0.8360 | 0.8360 | 1.0248 | 1.0363 | 1.0363 | 1.0572 |
| Change (Schiz- | | | | | | |
| Control) | | | | | | |
| DLPFC p- | 0.0444 | 0.0444 | 0.0103 | 0.0069 | 0.0069 | 0.0393 |
| value (Bipolar- | | | | | | |
| Control) | | | | | | |
| PFC Fold | 0.7931 | 0.7931 | 0.7490 | 0.7436 | 0.7436 | 1.2536 |
| Change | | | | | | |
| (Bipolar- | | | | | | |
| Control) | | | | | | |

Lymphocyte

| | | | | | | |
|---|---|---|---|---|---|---|
| p-value | 0.0000 | 0.0001 | 0.0000 | 0.0000 | 0.0000 | 0.0004 |
| (diagnosis) | | | | | | |
| p-value (Schiz- | 0.0000 | 0.0026 | 0.0000 | 0.0000 | 0.0000 | 0.0228 |
| Bipolar) | | | | | | |
| Mean (Schiz- | 1.7472 | 1.0863 | 1.6029 | 1.7234 | 1.5100 | 0.8611 |
| Bipolar) | | | | | | |
| p-value (Schiz- | 0.1739 | 0.4811 | 0.7570 | 0.2132 | 0.4108 | 0.4941 |
| Control) | | | | | | |
| Mean (Schiz- | 0.4277 | 0.2677 | −0.0854 | 0.3414 | 0.2625 | −0.2782 |
| Control) | | | | | | |
| p-value (Schiz- | 0.4664 | 0.6632 | 0.8419 | 0.7239 | 0.5756 | 0.5312 |
| Klinefelters) | | | | | | |
| Mean (Schiz- | 0.2276 | −0.1653 | −0.0550 | −0.0962 | −0.1783 | −0.2547 |
| Klinefelters) | | | | | | |
| p-value | 0.0000 | 0.0001 | 0.0000 | 0.0000 | 0.0000 | 0.0006 |
| (Klinefelters- | | | | | | |
| Bipolar) | | | | | | |
| Mean | 1.5196 | 1.2516 | 1.6579 | 1.8196 | 1.6883 | 1.1158 |
| (Klinefelters- | | | | | | |
| Bipolar) | | | | | | |
| p-value | 0.4557 | 0.1872 | 0.8980 | 0.0656 | 0.1109 | 0.9462 |
| (Klinefelters- | | | | | | |
| Control) | | | | | | |
| Mean | 0.2001 | 0.4330 | −0.0304 | 0.4376 | 0.4408 | −0.0235 |
| (Klinefelters- | | | | | | |
| Control) | | | | | | |
| p-value | 0.4664 | 0.6632 | 0.8419 | 0.7239 | 0.5756 | 0.5312 |
| (Klinefelters- | | | | | | |
| Schiz) | | | | | | |
| Mean | −0.2276 | 0.1653 | 0.0550 | 0.0962 | 0.1783 | 0.2547 |
| (Klinefelters- | | | | | | |
| Schiz) | | | | | | |
| p-value | 0.0000 | 0.0056 | 0.0000 | 0.0000 | 0.0000 | 0.0004 |
| (Bipolar- | | | | | | |
| Control) | | | | | | |
| Mean (Bipolar- | −1.3195 | −0.8185 | −1.6883 | −1.3820 | −1.2475 | −1.1393 |
| Control) | | | | | | |
| p-value | 0.0000 | 0.0001 | 0.0000 | 0.0000 | 0.0000 | 0.0006 |
| (Bipolar- | | | | | | |
| Klinefelters) | | | | | | |
| Mean (Bipolar- | −1.5196 | −1.2516 | −1.6579 | −1.8196 | −1.6883 | −1.1158 |
| Klinefelters) | | | | | | |
| p-value | 0.0000 | 0.0026 | 0.0000 | 0.0000 | 0.0000 | 0.0228 |
| (Bipolar- | | | | | | |
| Schiz) | | | | | | |
| Mean (Bipolar- | −1.7472 | −1.0863 | −1.6029 | −1.7234 | −1.5100 | −0.8611 |
| Schiz) | | | | | | |
| p-value | 0.0000 | 0.0001 | 0.0000 | 0.0000 | 0.0000 | 0.0006 |
| (Bipolar vs | | | | | | |
| KS) | | | | | | |

Lymphocyte Preparation (Fresh Lymphocyte-F, Tempus Whole Blood-
T, Transformed and passage-P)

| | | | | | | |
|---|---|---|---|---|---|---|
| p-value | 0.0000 | 0.0026 | 0.0000 | 0.0000 | 0.0000 | 0.0228 |
| (Bipolar vs Sz) | | | | | | |
| p-value | 0.4845 | 0.4845 | 0.6333 | 0.7101 | 0.7101 | 0.1459 |
| (Lymph_Prep) | | | | | | |
| sidak (p-value | 1.0000 | 1.0000 | 1.0000 | 1.0000 | 1.0000 | 1.0000 |
| (Lymph_Prep)) | | | | | | |
| p-value (F-P) | 0.5352 | 0.5352 | 0.4648 | 0.4606 | 0.4606 | 0.0651 |
| sidak (p- | 1.0000 | 1.0000 | 1.0000 | 1.0000 | 1.0000 | 1.0000 |
| value (F-P)) | | | | | | |
| Mean (F-P) | −0.1861 | −0.1861 | 0.1263 | 0.0939 | 0.0939 | 0.2320 |
| p-value (F-T) | 0.5451 | 0.5451 | 0.8595 | 0.9629 | 0.9629 | 0.1440 |
| sidak (p- | 1.0000 | 1.0000 | 1.0000 | 1.0000 | 1.0000 | 1.0000 |
| value (F-T)) | | | | | | |
| Mean (F-T) | 0.1754 | 0.1754 | −0.0294 | 0.0057 | 0.0057 | 0.1747 |
| p-value (P-T) | 0.2346 | 0.2346 | 0.3693 | 0.4878 | 0.4878 | 0.6345 |
| sidak (p- | 1.0000 | 1.0000 | 1.0000 | 1.0000 | 1.0000 | 1.0000 |
| value (P-T)) | | | | | | |
| Mean (P-T) | 0.3615 | 0.3615 | −0.1556 | −0.0883 | −0.0883 | −0.0574 |

Fold Change for both Lymphocyte and
DLPFC

| | | | | | |
|---|---|---|---|---|---|
| Down | Down | Down | Down | Down | Opposite |

| | Affymetrix | | | | |
|---|---|---|---|---|---|
| | Transcript | | | | |
| | cluster id | | | | |
| | 3770743 | 3592023 | 2434609 | 3204721 | 3918696 |
| Affymetrix | 215075_s_at | 201891_s_at | 202450_s_at | 204083_s_at | 213538_at |
| Probe Set ID | | | | | |
| Gene Symbol | GRB2 | B2M | CTSK | TPM2 | SON |
| Codelink Gene | GRB2 | B2M | CTSK | TPM2 | SON |
| Symbol | | | | | |

DLPFC

| | | | | | |
|---|---|---|---|---|---|
| DLPFC p- | 0.5779 | 0.7594 | 0.1034 | 0.8097 | 0.7153 |
| value (Schiz- | | | | | |
| Control) | | | | | |
| LPFC Fold | 1.0344 | 0.9784 | 0.8632 | 0.9763 | 0.9585 |
| Change (Schiz- | | | | | |
| Control) | | | | | |
| DLPFC p- | 0.0239 | 0.0055 | 0.0210 | 0.0263 | 0.0368 |
| value (Bipolar- | | | | | |
| Control) | | | | | |
| PFC Fold | 1.1497 | 0.8168 | 0.8103 | 0.7987 | 0.7822 |
| Change | | | | | |
| (Bipolar- | | | | | |
| Control) | | | | | |

Lymphocyte

| | | | | | |
|---|---|---|---|---|---|
| p-value | 0.0002 | 0.0000 | 0.0000 | 0.0000 | 0.0000 |
| (diagnosis) | | | | | |
| p-value (Schiz- | 0.0289 | 0.0000 | 0.0000 | 0.0000 | 0.0000 |
| Bipolar) | | | | | |
| Mean (Schiz- | 0.8153 | −1.4893 | −1.3340 | −1.4169 | −1.8035 |
| Bipolar) | | | | | |
| p-value (Schiz- | 0.2915 | 0.8956 | 0.8463 | 0.5439 | 0.3546 |
| Control) | | | | | |
| Mean (Schiz- | −0.4258 | −0.0346 | −0.0634 | 0.1966 | −0.2295 |
| Control) | | | | | |
| p-value (Schiz- | 0.4907 | 0.1532 | 0.2801 | 0.9134 | 0.3937 |
| Klinefelters) | | | | | |
| Mean (Schiz- | −0.2773 | 0.3806 | 0.3554 | −0.0352 | −0.2113 |
| Klinefelters) | | | | | |
| p-value | 0.0006 | 0.0000 | 0.0000 | 0.0000 | 0.0000 |
| (Klinefelters- | | | | | |
| Bipolar) | | | | | |
| Mean | 1.0926 | −1.8699 | −1.6895 | −1.3817 | −1.5922 |
| (Klinefelters- | | | | | |
| Bipolar) | | | | | |
| p-value | 0.6667 | 0.0712 | 0.1402 | 0.4053 | 0.9316 |
| (Kilnefelters- | | | | | |
| Control) | | | | | |
| Mean | −0.1485 | −0.4152 | −0.4189 | 0.2318 | −0.0182 |
| (Klinefelters- | | | | | |
| Control) | | | | | |
| p-value | 0.4907 | 0.1532 | 0.2801 | 0.9134 | 0.3937 |
| (Klinefelters- | | | | | |
| Schiz) | | | | | |
| Mean | 0.2773 | −0.3806 | −0.3554 | 0.0352 | 0.2113 |
| (Klinefelters- | | | | | |
| Schiz) | | | | | |
| p-value | 0.0001 | 0.0000 | 0.0000 | 0.0000 | 0.0000 |
| (Bipolar- | | | | | |
| Control) | | | | | |
| Mean (Bipolar- | −1.2411 | 1.4547 | 1.2706 | 1.6135 | 1.5740 |
| Control) | | | | | |
| p-value | 0.0006 | 0.0000 | 0.0000 | 0.0000 | 0.0000 |
| (Bipolar- | | | | | |
| Klinefelters) | | | | | |
| Mean (Bipolar- | −1.0926 | 1.8699 | 1.6895 | 1.3817 | 1.5922 |
| Klinefelters) | | | | | |
| p-value | 0.0289 | 0.0000 | 0.0000 | 0.0000 | 0.0000 |
| (Bipolar- | | | | | |
| Schiz) | | | | | |
| Mean (Bipolar- | −0.8153 | 1.4893 | 1.3340 | 1.4169 | 1.8035 |
| Schiz) | | | | | |
| p-value | 0.0006 | 0.0000 | 0.0000 | 0.0000 | 0.0000 |
| (Bipolar vs | | | | | |
| KS) | | | | | |

Lymphocyte Preparation (Fresh Lymphocyte-F, Tempus Whole Blood-T,
Transformed and passage-P)

| | | | | | |
|---|---|---|---|---|---|
| p- | 0.0289 | 0.0000 | 0.0000 | 0.0000 | |
| value (Bipolar | | | | | |
| vs Sz) | | | | | |
| p-value | 0.2142 | 0.7177 | 0.6862 | 0.1158 | 0.7202 |
| (Lymph_Prep) | | | | | |
| sidak (p-value | 1.0000 | 1.0000 | 1.0000 | 1.0000 | 1.0000 |
| (Lymph_Prep)) | | | | | |
| p-value (F-P) | 0.0889 | 0.9694 | 0.4985 | 0.1746 | 0.5356 |
| sidak (p- | 1.0000 | 1.0000 | 1.0000 | 1.0000 | 1.0000 |
| value (F-P)) | | | | | |
| Mean (F-P) | 0.1825 | −0.0026 | −0.1224 | 0.1987 | −0.1260 |
| p-value (F-T) | 0.2504 | 0.4696 | 0.8897 | 0.0437 | 0.8819 |
| sidak (p- | 1.0000 | 1.0000 | 1.0000 | 1.0000 | 1.0000 |
| value (F-T)) | | | | | |
| Mean (F-T) | 0.1167 | −0.0470 | 0.0241 | 0.2936 | 0.0291 |
| p-value (P-T) | 0.5262 | 0.5085 | 0.4191 | 0.5092 | 0.4470 |
| sidak (p- | 1.0000 | 1.0000 | 1.0000 | 1.0000 | 1.0000 |
| value (P-T)) | | | | | |
| Mean (P-T) | −0.0658 | −0.0445 | 0.1464 | 0.0949 | 0.1551 |

Fold Change for both Lymphocyte and
DLPFC

| | | | | |
|---|---|---|---|---|
| Opposite | Opposite | Opposite | Opposite | Opposite |

TABLE 10
A list of biomarker alterations associated with schizophrenia only.

| | Affymetrix Transcript_cluster_id | | |
|---|---|---|---|
| | 3300115 | 3853658 | 3494629 |
| | Affymetrix Probe Set ID | | |
| | 204284_at | 206153_at | 206884_s_at |
| | Gene Symbol | | |
| | PPP1R3C | CYP4F11 | SCEL |
| | Codelink Gene Symbol | | |
| | PPP1R3C | CYP4F11 | SCEL |
| DLPFC | | | |
| DLPFC p-value (Schiz-Control) | 0.0434 | 0.0367 | 0.0266 |
| DLPFC Fold Change (Schiz- | 1.3221 | 1.4224 | 1.6300 |
| Control) | | | |
| DLPFC p-value (Bipolar- | 0.1188 | 0.2424 | 0.2157 |
| Control) | | | |
| DLPFC Fold Change (Bipolar- | 1.2390 | 1.2148 | 1.3093 |
| Control) | | | |
| Lymphocyte | | | |
| p-value (diagnosis) | 0.0011 | 0.0027 | 0.0046 |
| p-value (Schiz-Bipolar) | 0.0005 | 0.0051 | 0.0011 |
| Mean (Schiz-Bipolar) | 1.3910 | 1.1311 | 1.3474 |
| p-value (Schiz-Control) | 0.0277 | 0.0004 | 0.0486 |
| Mean (Schiz-Control) | 0.9266 | 1.6210 | 0.8634 |
| p-value (Schiz-Klinefelters) | 0.0002 | 0.0014 | 0.0015 |
| Mean (Schiz-Klinefelters) | 1.6208 | 1.4429 | 1.4337 |
| p-value (Klinefelters-Bipolar) | 0.4563 | 0.3324 | 0.7884 |
| Mean (Klinefelters-Bipolar) | −0.2297 | −0.3118 | −0.0863 |
| p-value (Klinefelters-Control) | 0.0534 | 0.6279 | 0.1261 |
| Mean (Klinefelters-Control) | −0.6941 | 0.1781 | −0.5704 |
| p-value (Klinefelters-Schiz) | 0.0002 | 0.0014 | 0.0015 |
| Mean (Klinefelters-Schiz) | −1.6208 | −1.4429 | −1.4337 |
| p-value (Bipolar-Control) | 0.1354 | 0.1304 | 0.1364 |
| Mean (Bipolar-Control) | −0.4644 | 0.4900 | −0.4841 |
| p-value (Bipolar-Klinefelters) | 0.4563 | 0.3324 | 0.7884 |
| Mean (Bipolar-Klinefelters) | 0.2297 | 0.3118 | 0.0863 |
| p-value (Bipolar-Schiz) | 0.0005 | 0.0051 | 0.0011 |
| Lymphocyte Preparation (Fresh | | | |
| Lymphocyte-F, Tempus Whole Blood-T, | | | |
| Transfomed and Passage-P) | | | |
| Mean (Bipolar-Schiz) | −1.3910 | −1.1311 | −1.3474 |
| p-value (Lymph_Prep) | 0.2995 | 0.2989 | 0.8467 |
| sidak (p-value (Lymph_Prep)) | 1.0000 | 1.0000 | 1.0000 |
| p-value (F-P) | 0.8173 | 0.1248 | 0.8480 |
| sidak (p-value (F-P) | 1.0000 | 1.0000 | 1.0000 |
| Mean (F-P) | −0.0368 | 0.2037 | −0.0125 |
| p-value (F-T) | 0.2174 | 0.4495 | 0.7049 |
| sidak (p-value (F-T) | 1.0000 | 1.0000 | 1.0000 |
| Mean (F-T) | 0.1937 | 0.0947 | 0.0239 |
| p-value (P-T) | 0.1586 | 0.4016 | 0.5782 |
| sidak (p-value (P-T)) | 1.0000 | 1.0000 | 1.0000 |
| Mean (P-T) | 0.2305 | −0.1090 | 0.0364 |
| Fold Change for both | | | |
| Lymphocyte and DLPFC | | | |
| Direction | Up | Up | Up |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of detecting the presence of or a predisposition to a psychiatric illness in a human, said method comprising:
screening a biological sample from said human for increased or decreased expression of two or more genes listed in Table 6, where upregulation or downregulation (as indicated in Table 6) of expression of said two or more genes, is an indicator for the presence of, or predisposition to, a psychiatric illness.

2. The method of claim 1, wherein said psychiatric illness is schizophrenia and/or bipolar disorder.

3. The method of claim 1, wherein said screening comprises screening said biological sample for increased or decreased expression of three or more genes listed in Table 6.

4-5. (canceled)

6. The method of claim 1, wherein said screening comprises screening genes whose expression is concordant in DLPFC and lymphocytes.

7. The method of claim 1, wherein said two or more genes comprises:
two or more genes selected from the group consisting of BUB1B, ERBB2, FGF2, FTH1, IL2RA, LGALS3, MT1X, NFATC1, OGDH, PPARA, PVR, SOX9, SSPN, TXNIP, and UNG; and/or
two or more genes selected from the group consisting of EMX2, ERBB2, FGF2, JARID2, RAB23, SMO, SOX9, and THBS4; and/or
two or more genes selected from the group consisting of AGXT2L1, EMX2, SOX9, TU3A, TUBB2B, IMPA2, SLC1A2, GMPR, AHNAK, and ATP6V1H; and/or
two or more genes selected from the group consisting of BUB1B, EMX2, ERBB2, FGF2, FTH1, IL2RA, LGALS3, MAFG, NFATC1, PVR, RERG, SMCY, SMO, SOX9, TXNIP; and/or
two or more genes selected from the group consisting of CASP6, EPHB4, GLUL, HMGB2, MAOA, NOTCH2, SLC1A3, SLC6A8, TNFSF10, TNFSF8 and/or
two or more genes selected from the group consisting of CASP6, EPHB4, GLUL, HMGB2, MAOA, NOTCH2, SLC1A3, SLC6A8, TNFSF10, TNFSF8 and/or
two or more genes selected from the group consisting of HOMER1, MCCC2, CORT, and RGS4.

8-18. (canceled)

19. The method of claim 1, wherein said two or more genes comprises two or more genes selected from the group consisting of ATP6V1D, GSR, and SH3GLB1, and/or two or more genes selected from the group consisting of PPP1R3C, CYP4F11, and SCEL.

20. The method of claim 1, wherein said biological sample comprises a lymphocyte or a neurological tissue.

21. (canceled)

22. The method of claim 1, wherein said human is a human undergoing psychiatric evaluation and/or receiving psychoactive medication.

23-25. (canceled)

26. The method of claim 1, wherein said screening comprises a nucleic acid hybridization to determine an mRNA level of said two or more genes.

27-29. (canceled)

30. The method of claim 1, wherein said screening comprises detecting a protein(s) expressed by said two or more genes.

31-33. (canceled)

34. A method of distinguishing between schizophrenia and bipolar disorder or between a predisposition to schizophrenia and bipolar disorder in a human, said method comprising: screening a biological sample from said human for increased or decreased expression of two or more genes listed in Table 1, and/or Table 10, and/or Table 2, and/or Table 9, where dysregulation of the expression of the gene(s) as indicated in Table 1 or Table 10, as compared to a control, indicates the presence of, or a predisposition to schizophrenia, and dysregulation of the expression of the gene(s) as indicated in Table 2 or Table 9, as compared to a control, indicates the presence of or a predisposition to bipolar disorder.

35-37. (canceled)

38. The method of claim 34, wherein said screening comprises screening said biological sample for increased or decreased expression of two or more genes selected from the group consisting of ATP6V1D, GSR, and SH3GLB1, and/or two or more genes selected from the group consisting of PPP1R3C, CYP4F11, and SCEL.

39. The method of claim 34, wherein said biological sample comprises a lymphocyte or a neurological tissue.

40. (canceled)

41. The method of claim 34, wherein said human is a human undergoing psychiatric evaluation and/or receiving psychoactive medication.

42-44. (canceled)

45. The method of claim 34, wherein said screening comprises a nucleic acid hybridization to determine an mRNA level of said two or more genes and/or detecting a protein(s) expressed by said two or more genes.

46-53. (canceled)

54. A method of treating a human subject for a psychiatric disorder, said method comprising:
utilizing a biological sample from said human subject to detect the presence of or predisposition to a psychiatric illness in a said human according to claim 1; and
prescribing or providing more aggressive therapy for said human subject if said human subject tests positive for the presence or predisposition to a psychiatric illness; and/or prescribing treatment for schizophrenia for if said human subject tests positive for the presence or predisposition to schizophrenia, and/or or prescribing treatment for bipolar disorder for if said human subject tests positive for the presence or predisposition to bipolar disorder.

55-62. (canceled)

63. A method of screening for an agent that mitigates one or more symptoms of a psychiatric disorder, said method comprising:
administering a test agent to a cell and/or a mammal; and
detecting altered expression in said cell and/or mammal of two or more genes listed in Tables 1, 2, 6, 9, or 10, where upregulation or downregulation (as indicated in Tables 1, 2, 6, 9, or 10) of expression of said two or more genes, as compared to a control, is an indicator that said test agent has activity that mediates one or more symptoms of a psychiatric disorder.

64. The method of claim 63, wherein said psychiatric illness is schizophrenia and/or bipolar disorder.

65-70. (canceled)

71. The method of claim 63, wherein said two or more genes comprises:
two or more genes selected from the group consisting of BUB1B, ERBB2, FGF2, FTH1, IL2RA, LGALS3, MT1X, NFATC1, OGDH, PPARA, PVR, SOX9, SSPN, TXNIP, and UNG; and/or
two or more genes selected from the group consisting of EMX2, ERBB2, FGF2, JARID2, RAB23, SMO, SOX9, and THBS4; and/or
two or more genes selected from the group consisting of AGXT2L1, EMX2, SOX9, TU3A, TUBB2B, IMPA2, SLC1A2, GMPR, AHNAK, and ATP6V1H; and/or
two or more genes selected from the group consisting of BUB1B, EMX2, ERBB2, FGF2, FTH1, IL2RA, LGALS3, MAFG, NFATC1, PVR, RERG, SMCY, SMO, SOX9, TXNIP; and/or
two or more genes selected from the group consisting of HOMER1, MCCC2, CORT, and RGS4.

72-89. (canceled)
